(19) United States
(12) Patent Application Publication
(10) Pub. No.: US 2009/0130109 A1
(43) Pub. Date: May 21, 2009

(54) DCL-1 and uses thereof

(76) Inventors: Derek Nigel John Hart, Dutton Park (AU); Masato Kato, South Brisbane (AU)

Correspondence Address: KNOBBE MARTENS OLSON & BEAR LLP, 2040 MAIN STREET, FOURTEENTH FLOOR, IRVINE, CA, 92614, US

(21) Appl. No.: 11/888,911

(22) Filed: Jul. 31, 2007

Related U.S. Application Data
(63) Continuation-in-part of application No. 10/537,839, filed on May 18, 2006.

(30) Foreign Application Priority Data
Dec. 6, 2002 (AU) 2002953223

(51) Int. Cl. A61K 39/395; C12N 5/00; A61K 31/7105; A61K 31/711; A61P 37/02; A61P 37/00; C12Q 1/68; A61K 38/17

(52) U.S. Cl. 424/139.1; 435/375; 514/12; 514/44; 435/6

(57) ABSTRACT

The present invention relates generally to a novel lectin and to derivatives, homologues, analogues, chemical equivalents and mimetics thereof and, more particularly, to a novel type I C-type lectin, herein referred to as “DCL-1”. In particular, the present invention relates to the use of DCL-1 in therapeutic, prophylactic and diagnostic applications.

### CROSS-REFERENCE TO RELATED APPLICATIONS

This application us a Continuation-in-Part of U.S. application Ser. No. 10/537,839, which is a U.S. National Phase of International Application No. PCT/AU2003/001634, filed Dec. 5, 2003 and published in English, which claims priority to Australian Provisional Application No. 2002953223 filed Dec. 6, 2002. The entire contents of each and all these applications being hereby incorporated by reference herein in their entirety as if fully disclosed herein.

### FIELD OF THE INVENTION

The present invention relates generally to a novel lectin and to derivatives, homologues, analogues, chemical equivalents and mimetics thereof and, more particularly, to a novel type I C-type lectin, herein referred to as “DCL-1”. In particular, the present invention relates to the use of DCL-1 in therapeutic, prophylactic and diagnostic applications.

### BACKGROUND OF THE INVENTION

The reference to any prior art in this specification is not, and should not be taken as, an acknowledgment or any form of suggestion that that prior art forms part of the common general knowledge in Australia.

The C-type lectins represent a large family of Ca⁺⁺-dependent lectins that share primary structural homology in their carbohydrate-recognition domains. This very large family, which includes many endocytic receptors, many proteoglycans, and all known collectins and selecting, is found throughout the animal kingdom. Most of the members of this family differ, however, with respect to the types of carbohydrate structures that they recognize with high affinity. The C-type lectin family is diverse and is associated with many immune-system functions, such as inflammation and immunity to tumor and virally infected cells.

To date, more than 20 different proteins containing a C-type lectin carbohydrate-recognition domain have been identified in humans and corresponding homologs have also been found in many other higher animals. In addition, C-type lectins occur in many other vertebrates, including reptiles, and in invertebrates. From the genomic sequencing of Caenorhabditis elegans, approximately 150 C-type lectin genes have been identified. These many C-type lectins in higher animals are classified into subfamilies, based on their function or unique localization.

A growing list of proteins containing the C-type carbohydrate-recognition domain has been identified on human and rodent lymphocytes. For the most part, the functions of these proteins are poorly understood and their ability to bind carbohydrate has not been demonstrated.

DCL-1 (DEC-205-associated C-type lectin), also known as CD302, is a member of the C-type lectin receptor superfamily of cell surface proteins. DCL-1 is highly conserved amongst the human, mouse and rat homologues. The human DCL-1 gene, composed of 6 exons, is located in a cluster of type I transmembrane C-type lectin genes on chromosomal band 2q24. DCL-1 is known to be expressed by phagocytic white blood cells, which provide vital roles in innate and adaptive immune defenses.

### SUMMARY OF THE INVENTION

As explained in more detail below, the present invention is based on the discovery that DCL-1 behaves as an endocytic and phagocytic receptor and co-localizes with F-actin structures on filopodia, lamellipodia and podosomes. Thus the present inventors have determined that DCL-1 is involved in endocytosis, phagocytosis, cell adhesion and cell migration mediated by cells including immune cells such as antigen-presenting cells. Moreover, the inventors believe that DCL-1 as well as agonists and antagonists thereof can be used to modulate endocytosis, phagocytosis, cell adhesion and/or migration, and that these activities can be used as surrogate markers of DCL-1 level or activity. Thus, the present invention relates to the use of DCL-1 or a biologically fragment or derivative thereof, an antibody or fragment thereof which specifically binds thereto, a nucleic acid molecule encoding DCL-1 or a biologically active fragment or derivative thereof and/or a nucleic acid molecule antisense thereto to modulate endocytosis, phagocytosis, cell adhesion and/or cell migration mediated by cells including immune cells (e.g., antigen-presenting cells).

Moreover, the inventors hypothesize that DCL-1 or an antibody which specifically binds thereto, or a nucleic acid molecule encoding DCL-1 or a nucleic acid molecule antisense thereto, are targets for therapeutic manipulation and thus have use in the treatment, prevention and/or diagnosis of several diseases, including those associated with fungal or parasitic infections, cancer, hematologic and oncologic diseases, lymphoproliferative diseases, and diseases associated with transplantation, autoimmunity and inflammation.

Accordingly, in one aspect, the present invention provides a method for modulating an immune function of a cell that expresses DCL-1, the method comprising exposing the cell to an agent that modulates the level or functional activity of DCL-1, wherein the agent is selected from the group consisting of:
a) a proteinaceous molecule comprising an amino acid sequence which has at least 75% sequence identity to the sequence set forth in any one of SEQ ID NOs: 8, 12, 15 or 16 and which modulates at least one immune function selected from the group consisting of endocytosis, phagocytosis, cell adhesion and cell migration;
b) a proteinaceous molecule comprising an amino acid sequence which is encoded by a nucleotide sequence that hybridizes under high stringency conditions to the sequence set forth in any one of SEQ ID NOs: 7, 10, 11, 13, 14, 17 or 18 and which modulates at least one immune function selected from the group consisting of endocytosis, phagocytosis, cell adhesion and cell migration;
c) an antibody or fragment thereof which specifically binds to the amino acid sequence defined in a) or b);
d) a nucleic acid molecule comprising a nucleotide sequence that encodes the amino acid sequence defined in a) or b);
e) a nucleic acid molecule comprising a nucleic acid sequence that hybridizes under high stringency conditions to the nucleotide sequence defined in d); and
f) a nucleic acid molecule which comprises a nucleotide sequence that is antisense to the nucleotide sequence defined in d) or e).

In some embodiments, the immune function is selected from the group consisting of endocytosis, phagocytosis, cell adhesion and cell migration.

In some embodiments, the cell is an immune cell, which is suitably but not exclusively an antigen-presenting cell (e.g., dendritic cells and macrophages).

Another aspect of the present invention provides a method of modulating an immune response, comprising exposing a cell that expresses DCL-1 to an agent that modulates the level or functional activity of DCL-1, wherein the agent is selected from the group consisting of:
a) a proteinaceous molecule comprising an amino acid sequence which has at least 75% sequence identity to the sequence set forth in any one of SEQ ID NO: 8, 12, 15 or 16 and which modulates at least one immune function selected from the group consisting of endocytosis, phagocytosis, cell adhesion and cell migration;
b) a proteinaceous molecule comprising an amino acid sequence which is encoded by a nucleotide sequence that hybridizes under high stringency conditions to the sequence set forth in any one of SEQ ID NOs: 7, 10, 11, 13, 14, 17 or 18 and which modulates at least one immune function selected from the group consisting of endocytosis, phagocytosis, cell adhesion and cell migration;
c) an antibody or fragment thereof which specifically binds to the amino acid sequence defined in a) or b);
d) a nucleic acid molecule comprising a nucleotide sequence that encodes the amino acid sequence defined in a) or b);
e) a nucleic acid molecule comprising a nucleotide sequence that hybridizes under high stringency conditions to the nucleotide sequence defined in d);
f) a nucleic acid molecule which comprises a nucleotide sequence that is antisense to the nucleotide sequence defined in d) or e); and
g) an inhibitory RNA molecule that is specific to the nucleotide sequence defined in d) or e).

In another aspect the invention provides a method of treating or preventing a disease associated with an aberrant immune response in a subject, the method comprising administering to the subject an immune response-modulating effective amount of an agent that modulates the level or functional activity of DCL-1, wherein the agent is selected from the group consisting of:
a) a proteinaceous molecule comprising an amino acid sequence which has at least 75% sequence identity to the sequence set forth in any one of SEQ ID NOs: 8, 12, 15 or 16 and which modulates at least one immune function selected from the group consisting of endocytosis, phagocytosis, cell adhesion and cell migration;
b) a proteinaceous molecule comprising an amino acid sequence which is encoded by a nucleotide sequence that hybridizes under high stringency conditions to the sequence set forth in any one of SEQ ID NOs: 7, 10, 11, 13, 14, 17 or 18 and which modulates at least one immune function selected from the group consisting of endocytosis, phagocytosis, cell adhesion and cell migration;
c) an antibody or fragment thereof which specifically binds to the amino acid sequence defined in a) or b);
d) a nucleic acid molecule comprising a nucleotide sequence that encodes the amino acid sequence defined in a) or b);
e) a nucleic acid molecule comprising a nucleotide sequence that hybridizes under high stringency conditions to the nucleotide sequence defined in d);
f) a nucleic acid molecule which comprises a nucleotide sequence that is antisense to the nucleotide sequence defined in d) or e); and
g) an inhibitory RNA molecule that is specific to the nucleotide sequence defined in d) or e).

In another aspect the invention provides a method of treating or preventing a disease associated with an unwanted immune response in a subject, the method comprising administering to the subject the subject an immune response-modulating effective amount of an agent that modulates the level or functional activity of DCL-1, wherein the agent is selected from the group consisting of:
a) a proteinaceous molecule comprising an amino acid sequence which has at least 75% sequence identity to the sequence set forth in any one of SEQ ID NOs: 8, 12, 15 or 16 and which modulates at least one immune function selected from the group consisting of endocytosis, phagocytosis, cell adhesion and cell migration;
b) a proteinaceous molecule comprising an amino acid sequence which is encoded by a nucleotide sequence that hybridizes under high stringency conditions to the sequence set forth in any one of SEQ ID NOs: 7, 10, 11, 13, 14, 17 or 18 and which modulates at least one immune function selected from the group consisting of endocytosis, phagocytosis, cell adhesion and cell migration;
c) an antibody or fragment thereof which specifically binds to the amino acid sequence defined in a) or b);
d) a nucleic acid molecule comprising a nucleotide sequence that encodes the amino acid sequence defined in a) or b);
e) a nucleic acid molecule comprising a nucleotide sequence that hybridizes under high stringency conditions to the nucleotide sequence defined in d);
f) a nucleic acid molecule which comprises a nucleotide sequence that is antisense to the nucleotide sequence defined in d) or e); and
g) an inhibitory RNA molecule that is specific to the nucleotide sequence defined in d) or e).

In yet another aspect the invention provides the use of an agent that modulates the level or functional activity of DCL-1, wherein the agent is selected from the group consisting of:
a) a proteinaceous molecule comprising an amino acid sequence which has at least 75% sequence identity to the sequence set forth in any one of SEQ ID NOs: 8, 12, 15 or 16 and which modulates at least one immune function selected from the group consisting of endocytosis, phagocytosis, cell adhesion and cell migration;
b) a proteinaceous molecule comprising an amino acid sequence which is encoded by a nucleotide sequence that hybridizes under high stringency conditions to the sequence set forth in any one of SEQ ID NOs: 7, 10, 11, 13, 14, 17 or 18 and which modulates at least one immune function selected from the group consisting of endocytosis, phagocytosis, cell adhesion and cell migration;
c) an antibody or fragment thereof which specifically binds to the amino acid sequence defined in a) or b);
d) a nucleic acid molecule comprising a nucleotide sequence that encodes the amino acid sequence defined in a) or b);
e) a nucleic acid molecule comprising a nucleotide sequence that hybridizes under high stringency conditions to the nucleotide sequence defined in d);
f) a nucleic acid molecule which comprises a nucleotide sequence that is antisense to the nucleotide sequence defined in d) or e); and
g) an inhibitory RNA molecule that is specific to the nucleotide sequence defined in d) or e),
   in the treatment, prevention and/or diagnosis of a disease associated with an aberrant immune response.

In yet another aspect the invention provides the use of an agent that modulates the level or functional activity of DCL-1, wherein the agent is selected from the group consisting of:
a) a proteinaceous molecule comprising an amino acid sequence which has at least 75% sequence identity to the sequence set forth in any one of SEQ ID NOs: 8, 12, 15 or 16 and which modulates at least one immune function selected from the group consisting of endocytosis, phagocytosis, cell adhesion and cell migration;
b) a proteinaceous molecule comprising an amino acid sequence which is encoded by a nucleotide sequence that hybridizes under high stringency conditions to the sequence set forth in any one of SEQ ID NOs: 7, 10, 11, 13, 14, 17 or 18 and which modulates at least one immune function selected from the group consisting of endocytosis, phagocytosis, cell adhesion and cell migration;
c) an antibody or fragment thereof which specifically binds to the amino acid sequence defined in a) or b);
d) a nucleic acid molecule comprising a nucleotide sequence that encodes the amino acid sequence defined in a) or b);
e) a nucleic acid molecule comprising a nucleotide sequence that hybridizes under high stringency conditions to the nucleotide sequence defined in d);
f) a nucleic acid molecule which comprises a nucleotide sequence that is antisense to the nucleotide sequence defined in d) or e); and
g) an inhibitory RNA molecule that is specific to the nucleotide sequence defined in d) or e),
in the treatment, prevention and/or diagnosis of a disease associated with an unwanted immune response.

In some embodiments the disease is selected from cancers, infectious diseases and diseases associated with unwanted or deleterious immune responses.

In some embodiments the proteinaceous molecule or antibody or antibody fragment as broadly described above is coupled to, or otherwise associated with, an antigen that corresponds to at least a portion of a target antigen that associates with the disease.

In some embodiments the proteinaceous molecule comprises an amino acid sequence which has at least 80%, 85%, 90% or 95% sequence identity to the sequence set forth in any one of SEQ ID NOs: 8, 12, 15 or 16.

In another aspect the invention provides a method of screening an agent for ability to modulate an immune response, comprising:
contacting a cell expressing a nucleic acid molecule that comprises (a) a nucleotide sequence encoding an amino acid sequence which has at least 75% sequence identity to the sequence set forth in any one of SEQ ID NOs: 8, 12, 15 or 16 and which modulates at least one immune function selected from the group consisting of endocytosis, phagocytosis, cell adhesion and cell migration; or (b) a nucleotide sequence that hybridizes under high stringency conditions to the sequence set forth in any one of SEQ ID NOs: 7, 10, 11, 13, 14, 17 or 18 and which encodes an amino acid sequence that modulates at least one immune function selected from the group consisting of endocytosis, phagocytosis, cell adhesion and cell migration, with the agent; and
detecting a change in the level and/or activity of an expression product (e.g., transcript or polypeptide) of the nucleic acid molecule, relative to a normal or reference level and functional activity in the absence of the agent, wherein the change indicates that the agent modulates the immune response.

In some embodiments the nucleic acid molecule expressed by the cell of a) encodes an amino acid sequence which has at least 80%, 85%, 90% or 95% sequence identity to the sequence set forth in any one of SEQ ID NOs: 8, 12, 15 or 16.

Another aspect of the present invention provides a novel nucleic acid molecule in isolated form wherein said nucleic acid molecule comprises a novel DEC-205 intergenic splice variant.

In another aspect there is provided a novel nucleic acid molecule in isolated form wherein said nucleic acid molecule comprises a DEC-205/DCL-1 intergenic splice variant.

Yet another aspect provides a nucleic acid molecule or derivative, homologue or analogue thereof comprising a nucleotide sequence encoding an amino acid sequence substantially as set forth in SEQ ID NO: 2 or SEQ ID NO: 5 or a derivative, homologue or mimetic thereof having at least about 45% or greater similarity to at least 30 contiguous amino acids in SEQ ID NO: 2 or SEQ ID NO: 5.

Still another aspect provides a novel nucleic acid molecule or a derivative, homologue or analogue thereof in isolated form comprising a nucleotide sequence substantially as set forth in SEQ ID NO: 1 or SEQ ID NO: 4 or a nucleotide sequence having at least about 50% similarity to all or part thereof or a nucleotide sequence capable of hybridizing to the sequence set forth in SEQ ID NO: 1 or SEQ ID NO: 4 under low stringency conditions at 42° C.

Yet still another aspect of the present invention contemplates a nucleic acid molecule or derivative, homologue or analogue thereof comprising a nucleotide sequence substantially as set forth in SEQ ID NO:1 or SEQ ID NO:4 or a derivative thereof or capable of hybridizing to SEQ ID NO: 1 or SEQ ID NO:4 under low stringency conditions at 42° C. and which encodes an amino acid sequence corresponding to an amino acid sequence set forth in SEQ ID NO:2 or SEQ ID NO:5 or a sequence having at least about 45% similarity to at least 10 contiguous amino acids in SEQ ID NO:2 or SEQ ID NO:5.

Still yet another aspect of the present invention contemplates a nucleic acid molecule comprising a sequence of nucleotides substantially as set forth in SEQ ID NO: 1 or SEQ ID NO:4.

A further aspect of the present invention provides a novel cDNA or a derivative, homologue or analogue thereof in isolated form comprising a nucleotide sequence substantially as set forth in SEQ ID NO: 1 or SEQ ID NO: 4 or a nucleotide sequence having at least about 50% similarity to all or part thereof or a nucleotide sequence capable of hybridizing to the sequence set forth in SEQ ID NO: 1 or SEQ ID NO: 4 under low stringency conditions at 42° C.

Another further aspect of the present invention provides a nucleic acid molecule or derivative, homologue or analogue thereof comprising a nucleotide sequence encoding an amino acid sequence substantially as set forth in SEQ ID NO: 8 or a derivative, homologue or mimetic thereof having at least about 45% or greater similarity to at least 30 contiguous amino acids in SEQ ID NO: 8.

In another aspect there is provided a nucleic acid molecule or derivative, homologue or analogue thereof comprising a nucleotide sequence encoding an amino acid sequence substantially as set forth in SEQ ID NO: 12 or a derivative, homologue or mimetic thereof having at least about 45% or greater similarity to at least 30 contiguous amino acids in SEQ ID NO: 12.

In still another aspect there is provided a nucleic acid molecule or derivative, homologue or analogue thereof comprising a nucleotide sequence encoding an amino acid sequence substantially as set forth in SEQ ID NO: 15 or a derivative, homologue or mimetic thereof having at least about 45% or greater similarity to at least 30 contiguous amino acids in SEQ ID NO: 15.

In yet another aspect, the present invention provides a novel nucleic acid molecule or a derivative, homologue or analogue thereof in isolated form comprising a nucleotide sequence substantially as set forth in SEQ ID NO: 7 or a nucleotide sequence having at least about 50% similarity to all or part thereof or a nucleotide sequence capable of hybridizing to the sequence set forth in SEQ ID NO: 7 under low stringency conditions at 42° C.

In still yet another aspect, the present invention provides a novel nucleic acid molecule or a derivative, homologue or analogue thereof in isolated form comprising a nucleotide sequence substantially as set forth in SEQ ID NO: 11 or a nucleotide sequence having at least about 50% similarity to all or part thereof or a nucleotide sequence capable of hybridizing to the sequence set forth in SEQ ID NO: 11 under low stringency conditions at 42° C.

In still another aspect, the present invention provides a novel nucleic acid molecule or a derivative, homologue or analogue thereof in isolated form comprising a nucleotide sequence substantially as set forth in SEQ ID NO: 14 or a nucleotide sequence having at least about 50% similarity to all or part thereof or a nucleotide sequence capable of hybridizing to the sequence set forth in SEQ ID NO: 14 under low stringency conditions at 42° C.

A further aspect of the present invention contemplates a nucleic acid molecule or derivative, homologue or analogue thereof comprising a nucleotide sequence substantially as set forth in SEQ ID NO:7 or a derivative thereof capable of hybridizing to SEQ ID NO:7 under low stringency conditions at 42° C. and which encodes an amino acid sequence corresponding to an amino acid sequence set forth in SEQ ID NO:8 or a sequence having at least about 45% similarity to at least 30 contiguous amino acids in SEQ ID NO:8.

In another further aspect the present invention contemplates a nucleic acid molecule or derivative, homologue or analogue thereof comprising a nucleotide sequence substantially as set forth in SEQ ID NO: 11 or a derivative thereof capable of hybridizing to SEQ ID NO:11 under low stringency conditions at 42° C. and which encodes an amino acid sequence corresponding to an amino acid sequence set forth in SEQ ID NO: 12 or a sequence having at least about 45% similarity to at least 30 contiguous amino acids in SEQ ID NO:12.

In still another further aspect the present invention contemplates a nucleic acid molecule or derivative, homologue or analogue thereof comprising a nucleotide sequence substantially as set forth in SEQ ID NO:14 or a derivative thereof capable of hybridizing to SEQ ID NO:14 under low stringency conditions at 42° C. and which encodes an amino acid sequence corresponding to an amino acid sequence set forth in SEQ ID NOs:15 or 16 or a sequence having at least about 45% similarity to at least 30 contiguous amino acids in SEQ ID NOs:15 or 16.

Yet another further aspect of the present invention contemplates a nucleic acid molecule comprising a sequence of nucleotides substantially as set forth in SEQ ID NO:7, SEQ ID NO:11 or SEQ ID NO:14.

Still another further aspect of the present invention is directed to an isolated protein selected from the list consisting of:
(i) An isolated DEC-205 intergenic splice variant or a derivative, homologue, analogue, chemical equivalent or mimetic thereof.
(ii) An isolated DEC-205/DCL-1 intergenic splice variant or a derivative, homologue, analogue, chemical equivalent or mimetic thereof.
(iii) A protein having an amino acid sequence substantially as set forth in SEQ ID NO: 2 or SEQ ID NO: 5 or a derivative, homologue or mimetic thereof or a sequence having at least about 45% similarity to at least 30 contiguous amino acids in SEQ ID NO: 2 or SEQ ID NO: 5 or a derivative, homologue, analogue, chemical equivalent or mimetic of said protein.
(iv) A protein encoded by a nucleotide sequence substantially as set forth in SEQ ID NO: 1 or SEQ ID NO:4 or a derivative, homologue or analogue of said nucleotide sequence or a derivative, homologue, analogue, chemical equivalent or mimetic of said protein.
(v) A protein encoded by a nucleotide sequence substantially as set forth in SEQ ID NO: 1 or SEQ ID NO: 4 or a derivative, homologue or analogue thereof or a sequence encoding an amino acid sequence having at least about 45% similarity to at least 30 contiguous amino acids in SEQ ID NO: 2 or SEQ ID NO: 5 or a derivative, homologue, analogue, chemical equivalent or mimetic of said protein.
(vi) A protein encoded by a nucleic acid molecule capable of hybridizing to the nucleotide sequence set forth in SEQ ID NO:1 or SEQ ID NO:4 or a derivative, homologue or analogue thereof under low stringency conditions at 42° C. or a derivative, homologue, analogue, chemical equivalent or mimetic of said protein.
(vii) A protein encoded by a nucleic acid molecule capable of hybridizing to the nucleotide sequence as set forth in SEQ ID NO: 1 or SEQ ID NO:4 or a derivative, homologue or analogue thereof under low stringency conditions at 42° C. and which encodes an amino acid sequence substantially as set forth in SEQ ID NO:2 or SEQ ID NO:5 or a derivative, homologue or mimetic thereof or an amino acid sequence having at least about 45% similarity to at least 30 contiguous amino acids in SEQ ID NO:2 or SEQ ID NO:5.
(viii) A protein having an amino acid sequence substantially as set forth in SEQ ID NO: 8, SEQ ID NO: 12, or SEQ ID NOs: 15 or 16 or a derivative, homologue or mimetic thereof or a sequence having at least about 45% similarity to at least 30 contiguous amino acids in SEQ ID NO: 8, SEQ ID NO: 12, or SEQ ID NOs: 15 or 16 or a derivative, homologue, analogue, chemical equivalent or mimetic of said protein.
(ix) A protein encoded by a nucleotide sequence substantially as set forth in SEQ ID NOs: 7, 10, 11, 13, 14, 17 or 18 or a derivative, homologue or analogue of said nucleotide sequence or a derivative, homologue, analogue, chemical equivalent or mimetic of said protein.
(x) A protein encoded by a nucleotide sequence substantially as set forth in SEQ ID NOs: 7, 11 or 14 or a derivative, homologue or analogue thereof or a sequence encoding an amino acid sequence having at least about 45% similarity to at least 30 contiguous amino acids in SEQ ID NOs: 8, 12, 15 or 16 or a derivative, homologue, analogue, chemical equivalent or mimetic of said protein.
(xi) A protein encoded by a nucleic acid molecule capable of hybridizing to the nucleotide sequence set forth in SEQ ID NOs: 7, 10, 11, 13, 14, 17 or 18 or a derivative, homologue or analogue thereof under low stringency conditions at 42° C. or a derivative, homologue, analogue, chemical equivalent or mimetic of said protein
(xii) A protein encoded by a nucleic acid molecule capable of hybridizing to the nucleotide sequence as set forth in SEQ ID NOs:7, 11 or 14 or a derivative, homologue or analogue thereof under low stringency conditions at 42° C. and which encodes an amino acid sequence substantially as set forth in SEQ ID NOs:8, 12 or 15 or 16 or a derivative, homologue or mimetic thereof or an amino acid sequence having at least about 45% similarity to at least 30 contiguous amino acids in SEQ ID NOs:8, 12, 15 or 16.
(xiii) A protein as defined in any one of paragraphs (i) to (xii) in a homodimeric form.
(xiv) A protein as defined in any one of paragraphs (i) to (xii) in a heterodimeric form.

Another aspect of the present invention contemplates a method of modulating DEC-205 SV expression or DEC-205 SV functional activity in a mammal, said method comprising administering to said mammal an agent for a time and under conditions sufficient to up-regulate, down-regulate or otherwise modulate expression of DEC-205 SV or functioning of DEC-205 SV.

Yet another aspect of the present invention is directed to a method for modulating DCL-1 expression or DCL-1 functional activity in a mammal, said method comprising administering to said mammal an agent for a time and under conditions sufficient to up-regulate, down-regulate or otherwise modulate said expression or functioning.

Still another aspect of the present invention contemplates a method for regulating cellular activity in a subject said method comprising administering to said subject an effective amount of an agent for a time and under conditions sufficient to modulate DEC-205 SV expression of DEC-205 SV functional activity.

In yet another aspect there is contemplated a method of regulating cellular activity in a subject said method comprising administering to said subject an effective amount of an agent for a time and conditions sufficient to modulate DCL-1 expression or DCL-1 functional activity.

In yet still another aspect there is provided a method for the treatment and/or prophylaxis of a condition characterized by aberrant, unwanted or otherwise inappropriate functioning of DEC-205 SV or DCL-1 in a subject, said method comprising administering to said subject an effective amount of an agent as hereinbefore defined for a time and under conditions sufficient to modulate the expression of DEC-205 SV or DCL-1 and/or functioning of DEC-205 SV or DCL-1.

In still yet another aspect there is provided a method for the treatment of Hodgkin's lymphoma in a mammal, said method comprising administering to said mammal an effective amount of a cytolytic and/or cytotoxic agent which agent interacts or otherwise associates with DEC-205 SV, for a time and under conditions sufficient for said agent to lyse, apoptose or otherwise kill Hodgkin and Reed-Sternberg cells.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 (A-C) shows a DCL-1 protein and gene comparison amongst different species. (A) Amino acid comparison of human (h), mouse (m) and rat (r) DCL-1 homologues. Identical amino acids are shown in dashes. Conservatively substituted amino acids are highlighted in grey. Deleted amino acids are shown in dots. Conserved cysteine and acidic amino acids are indicated by asterisks and open circles, respectively. Conserved serine/threonine and tyrosine phosphorylation sites are shown in open and closed diamonds, respectively. A potential N-glycosylation site is boxed. Putative endocytosis and late endosome-targeting signals are single and double-underlined, respectively. SP, signal peptide; CTLD, C-type lectin like domain; TM, transmembrane domain; CP cytoplasmic domain. The bold bars indicate untranslated regions. (B) DCL-1 gene structural comparison between human, mouse and rat. In the top panel, boxes indicate structural domains of DCL-1 protein in the complete DCL-1 mRNA. In the lower panels, black boxes and horizontal lines indicate exons and introns, respectively. Numbers indicate exon numbers. Hatched lines indicate exons encoding DCL-1 domains. The chromosomal localization is shown in brackets. (C) C-type lectin gene cluster comparison among human, mouse and rat. Black boxes and horizontal lines indicate C-type lectin genes and intergenic sequences, respectively.

FIG. 2 (A-C) shows a BLAST2 analyses of the human (h), mouse (m) and rat (r) homologs. (A) hDCL-1 (SEQ ID NO: 23) aligned with mDCL-1 (SEQ ID NO: 26). (B) hDCL-1 (SEQ ID NO: 23) aligned with rDCL-1 (SEQ ID NO: 24). (C) mDCL-1 (SEQ ID NO: 27) aligned with rDCL-1 (SEQ ID NO: 25).

FIG. 3 (A-C) shows characterization of hDCL-1 protein expressed in tCHO-hDCL-1 transfectants. (A) Surface hDCL-1 protein expressed on HB12 cells was detected with anti-FLAG mAb M2 by flow cytometry (bold line). The grey fill indicates an isotype control staining. (B) Characterization of hDCL-1 protein expressed by HB12 cells. Cell lysate from HB12 cells was immunoprecipitated with anti-hDCL-1 cytoplasmic domain (DCL-1 CP) or preimmune rabbit antibody (Preimmune) and protein A beads, fractionated by SDS-PAGE in reducing (+DTT) or non-reducing conditions (−DTT), followed by Western blotting with anti-FLAG mAb M2 and HRP-conjugated goat anti-mouse IgG. The signals were detected by enhanced chemiluminescence. Asterisks indicate non-specific bands. (C) Effect of N-glycosidase F digestion on HB12-derived hDCL-1 protein. The DCL-1 protein immunoprecipitated from HB12 was digested with N-glycosidase F (N-glyase F) and subjected to Western blot analysis as above. The positions of molecular mass standards are shown on the right. IP, immunoprecipitation; WB, Western blotting.

FIG. 4 (A-B) shows expression of hDCL-1 mRNA. (A) Expression of hDCL-1 mRNA in multiple tissues. The multiple tissue expression array was probed with [³²P]hDCL-1 cDNA and the signals were detected by scintillation counting. (B) Expression of hDCL-1 mRNA in leukocytes. FACS purified leukocytes (purity>98%) and monocyte-derived DC and macrophages (Mph) were subjected to semi-quantitative RT-PCR for hDCL-1 mRNA expression and fractionated by agarose gel electrophoresis. GAPDH was used to normalize the cDNA input.

FIG. 5 (A-C) shows production and characterization of monoclonal antibodies against hDCL-1 protein. (A) HB12 cells were stained with a series of anti-hDCL-1 mAb (MMRI-18, 19, 20 and 21) and subjected to flow cytometry analysis. The grey fills indicate an isotype control staining. (B) Immunoprecipitation of hDCL-1 protein from PBMC using the anti-DCL-1 mAb. Cell surface biotinylated PBMC lysate was immunoprecipitated with the anti-hDCL-1 mAb or an isotype control IgG₁ (Ctr IgG₁), subjected to Western blotting in non-reduced conditions. The positions of molecular mass standards are shown on the left. Arrows indicate the specific hDCL-1 protein bands. (C) Inhibition of PE-conjugated MMRI-19 and FITC-conjugated MMRI-20 binding to HB12 by unconjugated anti-hDCL-1 mAb. HB12 cells were preincubated with unconjugated anti-DCL-1 mAb (10 μg/ml), stained with PE-conjugated MMRI-19 (left panel) and FITC-conjugated MMRI-20 (right panel) and their binding detected by flow cytometry. An isotype control IgG₁ (Ctr IgG₁) was used as negative control.

FIG. 6 (A-C) shows expression of hDCL-1 on human leukocytes by flow cytometry. (A) Blood leukocytes and lineage negative cells. PBMC were stained with FITC-MMRI-20 in combination with lineage markers as described in Materials and Methods. (B) Expression of hDCL-1 on monocyte-derived Mph. Mph differentiated from CD14+ Mo with CSF-1 were incubated without (Mph) or with LPS (Act Mph) and stained with FITC-MMRI-20. (C) Expression of hDCL-1 on MoDC. MoDC differentiated from CD14+Mo with GM-CSF and IL-4 were incubated without (Mph) or with LPS (Act Mph) and stained with FITC-MMRI-20. The bold line and grey fill indicate MMRI-20 staining and an isotype control staining, respectively.

FIG. 7 (A-B) shows detection of hDCL-1 in leukocyte lysate by immunoprecipitation/Western blot analysis. (A) Cell lysate from FACS-purified leukocytes, monocyte-derived Mph and MoDC (400 and 133 μg/ml, indicated by black triangles) was immunoprecipitated with anti-hDCL-1 cytoplasmic domain ((DCL-1 CP) or pre-immune rabbit antibody (Preimmune) and protein A beads, fractionated by SDS-PAGE in non-reducing conditions, followed by Western blotting with MMRI-20. HB12 cells were used as a positive control. (B) FACS purified leukocytes, monocyte-derived Mph and MoDC were cell surface-biotinylated and lysed in a lysis buffer. The cell lysate containing equal amount of protein (100 μg/ml) was immunoprecipitated with MMRI-20 or an isotype control antibody (Ctr IgG₁) and protein G beads, fractionated by SDS-PAGE in reducing conditions, followed by Western blotting with HRP-conjugated streptavidin and enhanced chemiluminescence detection for short (10 min) and long exposures (16 h). HB12 cells were used as a positive control. Arrows indicate the specific hDCL-1 protein bands. The positions of protein molecular mass standards are shown on the right. Asterisks indicate non-specific bands. Arrowheads indicate potential hDCL-1-associated proteins, coimmunoprecipitated with hDCL-1. IP, immunoprecipitation; WB, Western blotting.

FIG. 8 (A-C) shows hDCL-1 colocalizes with F-actin and is internalized when bound with hDCL-1 mAb in HB12 cells. (A) Colocalization of hDCL-1 with F-actin in HB12 cells. The cells cultured on cover slips were fixed with PFA, permeabilized and stained with MMRI-21 and AF₄₈₈₋GAM (green), followed by counterstained with Texas red-phalloidin (red) and DAPI (blue). The cells were analyzed by LSM with x-y-z sectioning using a 100× objective. Top panels, x-y sectioning at basal cell surface; bottom panels, x-z sectioning. (B) hDCL-1 internalization by HB12 cells by flow cytometry. The cells were incubated with FITC-conjugated MMRI-20 or an isotype control IgG₁ (Ctr IgG₁) at 37° C. for various time periods. At the time points, cells were chilled, harvested in cold MACS buffer and stained with biotinylated MMRI-21 followed by APC-conjugated streptavidin for flow cytometry. (C) hDCL-1 internalization by HB12 cells by LSM. The cells cultured on cover slips were incubated with FITC-conjugated (green) MMRI-20 (top two rows) or an isotype control mAb (bottom two rows) as in (B). At the time points, cells were chilled on ice, stained with biotinylated MMRI-21 and AF₆₃₃-streptavidin (blue), and fixed with PFA. After permeabilization, the cells were counterstained with AF₅₄₆-phalloidin (red) and DAPI, and analyzed by LSM with x-y sectioning at basal cell surface using a 100× objective. For simplicity, selected time points (0 and 30 min) are shown. DAPI staining is omitted.

FIG. 9 (A-B) shows HB12 cells bind and phagocytose MMRI-20-coated microbeads specifically. (A) Rat anti-mouse IgG-conjugated microbeads (4.5 μm in diameter) were coated with MMRI-20 or an isotype control IgG₁ (Ctr IgG₁), and incubated with the clone HB12 in on ice. After washing to remove unbound microbeads, the cells were harvested with cold MACS buffer, stained with AF₄₈₈-GAM and the binding of the microbeads analyzed by flow cytometry. (B) The clone HB12 cells cultured on cover slips were incubated with the mAb-coated microbeads on ice as above. After washing, the cells were replenished with the tissue culture medium and incubated for various time periods. At the time points, the cells were chilled on ice, fixed with PFA. After permeabilization, the cells were stained with AF₄₈₈-GAM (green), AF₅₄₆-phalloidin (red) and DAPI (blue), and analyzed by LSM with x-y-z sectioning using a 100× objective. Large panels, x-y sectioning at centers of nuclei; horizontal strips, x-z sectioning; vertical strips, y-z sectioning. White arrowheads indicate phagocytic cup or phagosomes. White arrows indicate mouse IgG dissociated from the microbeads.

FIG. 10 (A-C) shows that human monocyte-derived Mph preferably bind anti-MMR and anti-DEC-205 mAb-coated microbeads, but not anti-hDCL-1-coated microbeads. (A) Cell surface expression of hDCL-1, MMR and DEC-205 on Mph detected with a quantitative indirect immunofluorescence analysis. Mph differentiated from CD14+Mo with CSF-1 were stained with a supersaturating concentration (20 μg/ml) of MMRI-20 (anti hDCL-1), mAb 15-2 (anti MMR) or MMRI-7 (anti DEC-205) and FITC-GAM, and analyzed by FACS. Grey fills indicate an isotype control IgG₁ staining. The numbers in brackets indicate the number of specific antibody binding sites in the Mph preparation determined by the assay. (B) Rat anti-mouse IgG-conjugated microbeads (4.5 μm in diameter) were coated with MMRI-20, mAb 15-2, MMRI-7 or the isotype control IgG₁ (Ctr IgG₁). Monocyte-derived Mph cultured on cover slips were incubated with the mAb-coated microbeads on ice, washed to remove unbound microbeads and fixed with PFA. After permeabilization, the cells were stained with AF₄₈₈-GAM (green), AF₅₄₆-phalloidin (red) and DAPI (blue), and analyzed by LSM using a 20× objective. The inserts correspond to magnified views of boxed areas. (C) Quantitation of mAb-coated microbeads binding to Mph. The number of mAb-coated microbeads and the number of cells were counted from randomly selected confocal microscopic fields (10 fields for anti-hDCL-1, anti-MMR/CD206 and anti-DEC-205 and 20 fields for the isotype control IgG₁) and expressed as number of microbeads/cell (mean±SD).

FIG. 11 (A-C) shows that hDCL-1 colocalizes with F-actin cytoskeletons in human monocyte-derived Mph and COS-1 cells expressing the hDCL-1-EGFP fusion protein. Mph cultured on cover slips were treated with DMSO (a solvent control) (A) or with cytochalasin D (B) for 30 min at 37° C., fixed and permeabilized. The cells were stained with MMRI-20, mAb 15-2, MMRI-7 or an isotype control mAb, followed by AF₄₈₈-conjugated donkey anti mouse IgG (green), counter stained with AF₅₄₆-phalloidin (red) and DAPI, and analyzed by LSM at basal surface levels using a 100× objective. (C) COS-1 cells were transiently transfected with pEGFP-hDCI-1 (left panels) or pEGFP-N1 (right panels) for 24 h. The cells were fixed with PFA, permeabilized and stained with AF₅₄₆-phalloidin (red) and DAPI, and analyzed by LSM. Arrows and arrowheads indicate colocalization of DCL-1-EGFP and F-actin at microvilli on the apical surface and at cell cortex, respectively. Asterisks indicate newly synthesized DCL-1-EGFP in endoplasmic reticulum and/or Golgi apparatus. For simplicity, DAPI staining is omitted.

FIG. 12 shows comparisons of C-type lectin (like) domain sequences of human DCSIGN/CD209 (SEQ ID NO: 28), MGL/CD301 (SEQ ID NO: 29), MMR/CD206 CRD4 (SEQ ID NO: 30) and hDCL-1 (SEQ ID NO: 31). Symbols above sequences represents consensus residues found in functional C-type lectin domains (3, 5). χ=aliphatic or aromatic (FWYHLIV), φ=aliphatic (LIV), o=aromatic (FWYH), *=side chain with carbonyl oxygen (DNEQ), Z=E or Q, B=D or N. Numbers indicate binding sites for auxiliary (site 1) and principal Ca²⁺ (site 2) binding. Conserved residues within sequences are highlighted. The single and double underlines indicate the position of EPN/QPD and WND motif, respectively.

### DETAILED DESCRIPTION OF THE PREFERRED EMBODIMENT

Before describing the invention in detail it is to be understood that it is not limited to particularly exemplified methods, formulations, or components and may, of course, vary. It is also to be understood that the terminology used herein is for the purpose of describing particular embodiments of the invention only, is not intended to be limiting, and will be limited only by the appended claims.

All publications, patents and patent applications cited herein, whether above or below, are hereby incorporated by reference in their entirety. However, publications mentioned herein are cited for the purpose of describing and disclosing the protocols and reagents which are reported in the publications and which might be used in connection with the invention. Nothing herein is to be construed as an admission that the invention is not entitled to antedate such disclosure by virtue of prior invention.

Furthermore, the practice of the present invention employs, unless otherwise indicated, conventional pharmaceutical and medical techniques within the skill of the art. Such techniques are well known to the skilled worker, and are explained fully in the literature. See, e.g., “Molecular Cloning: A Laboratory Manual, 2^{nd} Ed” Sambrook et al, Cold Spring Harbor Laboratory, Cold Spring Harbor, N.Y. (1989); “Current Protocols in Molecular Biology” Ausubel et al, eds., John Wiley & Sons, Inc, 1995; “Remington's Pharmaceutical Sciences”, 17^{th} Edition, Mack Publishing Company, Easton, Pa., USA.

It must be noted that, as used in the subject specification, the singular forms “a”, “an” and “the” include plural aspects unless the context clearly dictates otherwise. Thus, for example, reference to a “molecule” includes a single molecule as well as two or more molecules, an antibody refers to one or more antibodies, a cell refers to one or more cells, and the like.

Throughout the specification the word “comprise” and variations of the word, such as “comprising” and “comprises”, means “including but not limited to” and is not intended to exclude other additives, components, integers or steps. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

The subject specification contains amino acid and nucleotide sequence information prepared using the program PatentIn Version 3.1, presented herein after the bibliography. Each nucleotide sequence is identified in the sequence listing by the numeric indicator <201> followed by the sequence identifier (e.g., <210>1, <210>2, etc). The length, type of sequence (DNA, etc) and source organism for each nucleotide sequence is indicated by information provided in the numeric indicator fields <211>, <212> and <213>, respectively. Nucleotide sequences referred to in the specification are identified by the indicator SEQ ID NO: followed by the sequence identifier (e.g., SEQ ID NO: 1, SEQ ID NO: 2, etc.). The sequence identifier referred to in the specification correlates to the information provided in numeric indicator field <400> in the sequence listing, which is followed by the sequence identifier (e.g., <400>1, <400>2, etc). That is SEQ ID NO: 1 as detailed in the specification correlates to the sequence indicated as <400>1 in the sequence listing. A summary of the sequences detailed in this specification are provided immediately prior to the examples, in Table 1.

Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

The present invention is based on the discovery that DCL-1 behaves as an endocytic and phagocytic receptor and co-localizes with F-actin structures on filopodia, lamellipodia and podosomes. Thus the inventors have determined that DCL-1 is involved in endocytosis, phagocytosis, cell adhesion and cell migration. Moreover, the present inventors believe that DCL-1 and agonists and antagonists thereof can be used to modulate endocytosis, phagocytosis, cell adhesion and/or migration mediated by cells expressing DCL-1, including immune cells (e.g., antigen-presenting cells). Thus, the present invention relates to the use of DCL-1 or a biologically fragment or derivative thereof, an antibody or fragment thereof which specifically binds thereto, a nucleic acid molecule encoding DCL-1 or a biologically active fragment or derivative thereof and/or a nucleic acid molecule antisense thereto to modulate endocytosis, phagocytosis, cell adhesion and/or cell migration.

Furthermore, the discovery that DCL-1 is associated with F-actin has led the inventors to hypothesize that DCL-1 is involved in hematopoiesis, leukocyte trafficking and phagocytic leukocyte immune effector functions. This hypothesis has been made on the basis that contact between DCL-1 and its ligand(s) on other cells or tissue matrix may directly or indirectly control any one or more of the growth, differentiation, activation and/or migration of hematopoietic stem cells, committed leukocyte progenitors and leukocyte populations.

Therefore the inventors believe that DCL-1 is a target for therapeutic manipulation and thus has use in the treatment, prevention and/or diagnosis of several diseases, including those associated with pathogenic infections including fungal or parasitic infections, cancer, hematologic and oncologic diseases, lymphoproliferative diseases, and diseases associated with transplantation, autoimmunity and inflammation.

As used herein, “DCL-1” includes and encompasses a protein comprising the sequence shown in any of SEQ ID NOs: 8, 12, 15 and 16 as well as proteins that display substantial sequence similarity or identity to the sequence shown in any of SEQ ID NOs: 8, 12, 15 and 16, as described in more detail below. A human DCL-1 sequence is provided herein by the amino acid sequence set forth in SEQ ID NO:8, mouse DCL-1 sequence is provided herein by the amino acid sequence set forth in SEQ ID NO:12 and rat DCL-1 sequence is provided herein by the amino acid sequence set forth in SEQ ID NO: 16.

The term “protein” should be understood to encompass peptides, polypeptides and protein. The protein may be glycosylated or unglycosylated and/or may contain a range of other molecules fused, linked, bound or otherwise associated to the protein such as amino acids, lipids, carbohydrates or other peptides, polypeptides or proteins. Reference hereinafter to a “protein” includes a protein consisting of a sequence of amino acids as well as a protein associated with another molecules, such as an amino acid, lipid, carbohydrate or other peptide, polypeptide or protein.

In some embodiments a biologically active fragment or derivative of DCL-1 may be used. The term “fragment” means a portion of an entire molecule. A “biologically active fragment” is one which retains a biological activity of the full-length molecule. Thus a biologically active fragment of DCL-1 is a portion of the full-length DCL-1 protein which is involved in the modulation of endocytosis, phagocytosis, cell adhesion and/or cell migration.

“Derivatives” of the DCL-1 protein include homologues and analogues of DCL-1. Derivatives may be derived by insertion, deletion or substitution of amino acids. Insertional amino acid sequence derivatives are those in which one or more amino acid residues are introduced into a predetermined site in the DCL-1 protein although random insertion is also possible with suitable screening of the resulting product. Amino acid insertional derivatives include amino and/or carboxylic terminal fusions as well as intrasequence insertions of single or multiple amino acids. Deletional derivatives are characterized by the removal of one or more amino acids from the DCL-1 sequence. Substitutional amino acid derivatives are those in which at least one residue in a sequence has been removed and a different residue inserted in its place. The percentage similarity between DCL-1 and a derivative thereof may be greater than 45% such as at least 50% or at least 55% or at least 60% or at least 65% or at least 70% or at least 75% or at least 80% or at least 85% or at least 90%, 91%, 92%, 93%, 94%, 95%, 96%, 97%, 98%, 99% or higher.

The term “similarity” as used herein includes exact identity between compared amino acid sequences. Where there is non-identity at the amino acid level, “similarity” includes amino acids that are nevertheless related to each other at the structural, functional, biochemical and/or conformational levels. To determine the percent identity of two amino acid sequences, the sequences may be aligned for optimal comparison purposes (e.g., gaps can be introduced in the sequence of a first amino acid sequence for optimal alignment with a second amino acid sequence). The amino acid residues at corresponding positions can then be compared. When a position in the first sequence is occupied by the same amino acid residue as the corresponding position in the second sequence, then the molecules are identical at that position. The percent identity between the two sequences is a function of the number of identical positions shared by the sequences (i.e. % identity=# of identical positions/total # of overlapping positions×100). Preferably, the two sequences are the same length. The determination of percent identity or homology between two sequences can be accomplished using a mathematical algorithm. A suitable, mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90.5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to the nucleic acid molecules of the invention. BLAST protein searches can be performed with XBLAST program, score=50, wordlength=3 to obtain amino acid sequences homologous to the protein molecules of the invention. To obtain gapped alignments for comparison purposes, Gapped BLAST can be utilized as described in Altschul et al. (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov. Another example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, only exact matches are counted. Yet another example of a suitable algorithm is one such Gap which considers all possible alignment and gap positions and creates an alignment with the largest number of matches bases and the fewest gaps. Gap uses the alignment method of Needleman and Wunsch. Gap reads a scoring matrix that contains values for ever possible GCG symbol match. GAP is available on ANGIS (Australian National Genomic Information Service) at website http://mel1.angis.org.au.

The skilled person will appreciate that the term “similarity” may also be applied to nucleic acid sequences. In this case, where there is non-identity at the nucleotide level “similarity” includes differences between sequences which result in different amino acids that are nevertheless related to each other at the structural, functional, biochemical and/or conformational levels.

By “homologue” is meant DCL-1 or a biologically active fragment or derivative thereof derived from a species other than human. For example, the homologue may be a molecule derived from a non-human primate, livestock animal (e.g. sheep, pig, cow, horse, donkey), laboratory test animal (e.g. mouse, rabbit, rat, guinea pig), companion animal (e.g. dog, cat), captive wild animal (e.g. fox, kangaroo, deer), aves (e.g. chicken, geese, duck, emu, ostrich), reptile or fish.

“Analogues” of DCL-1 or a biologically active fragment thereof include, but are not limited to, molecules having modified side chains, incorporating unnatural amino acids and/or their derivatives during peptide, polypeptide or protein synthesis and the use of crosslinkers and other methods which impose conformational constraints on the proteinaceous molecules or their analogues.

DCL-1 or a biologically active fragment or derivative thereof may be in multimeric form meaning that two or more molecules are associated together. Where the same DCL-1 proteins, or biologically active fragments or derivatives thereof, are associated together, the complex is a homomultimer. An example of a homomultimer is a homodimer. Where at least one DCL-1 protein or biologically active fragment or derivative thereof is associated with at least one non-DCL-1 molecule, then the complex is a heteromultimer such as a heterodimer.

“DCL-1” also includes DCL-1 or a biologically active fragment or derivative thereof having a particular epitope or part of the entire protein fused to a peptide, polypeptide or other proteinaceous or non-proteinaceous molecule at the N- and/or C-terminus. For example, DCL-1 or a fragment or derivative thereof may be fused, linked or coupled to a molecule to tag to facilitate screening and/or purification of DCL-1 or conjugated to a molecule to facilitate its homing to a cell.

The DCL-1 protein or a biologically active fragment or derivative thereof is preferably in isolated form. By “isolated” is meant a molecule, such as a protein or nucleic acid molecule, having undergone at least one purification step and this is conveniently defined, for example, by a composition comprising at least about 10% subject molecule, preferably at least about 20%, more preferably at least about 30%, still more preferably at least about 40-50%, even still more preferably at least about 60-70%, yet even still more preferably 80-90% or greater of subject molecule relative to other components as determined by molecular weight, sequence or other convenient means. The molecule may also be considered in some embodiments to be biologically pure.

DCL-1 or a biologically active fragment or derivative thereof may be obtained from either a natural or a non-natural source. Non-natural sources include, for example, recombinant or synthetic sources. By “recombinant sources” is meant that the cellular source from which the subject molecule is harvested has been genetically altered. This may occur, for example, in order to increase or otherwise enhance the rate and volume of production by that particular cellular source.

The ability to produce recombinant DCL-1 or a biologically active fragment or derivative thereof permits the large scale production of these molecules for commercial use. The DCL-1 molecule or biologically active fragment or derivative thereof may need to be produced as part of a large peptide, polypeptide or protein which may be used as is or may first need to be processed in order to remove the extraneous proteinaceous sequences. Such processing includes digestion with proteases, peptidases and amidases or a range of chemical, electrochemical, sonic or mechanical disruption techniques.

Alternatively, chemical synthetic techniques may be used in the synthesis of DCL-1 or a biologically active fragment or derivative thereof. DCL-1 or a biologically active fragment or derivative thereof may be conveniently synthesized based on molecules isolated from a mammal. Isolation of these molecules may be accomplished by any suitable means such as by chromatographic separation, for example using CM-cellulose ion exchange chromatography followed by Sephadex (e.g. G-50 column) filtration. Many other techniques are available including HPLC and PAGE amongst others.

DCL-1 or a biologically active fragment or derivative thereof may be synthesized by solid phase synthesis using F-moc chemistry as described by Carpino et al. (1991). DCL-1 or a biologically active fragment or derivative thereof may also be synthesized by alternative chemistries including, but not limited to, t-Boc chemistry as described in Stewart et al. (1985) or by classical methods of liquid phase peptide synthesis.

The inventors have found that DCL-1 or a biologically active fragment or derivative thereof modulates endocytosis, phagocytosis, cell adhesion and/or cell migration. Therefore antibodies, including catalytic antibodies, which specifically bind DCL-1 or a fragment or derivative thereof, may also modulate endocytosis, phagocytosis, cell adhesion and/or cell migration mediated by cells expressing DCL-1, including immune cells (e.g., antigen-presenting cells). For example, an antibody which specifically binds DCL-1 would be expected to down-regulate endocytosis, phagocytosis, cell adhesion and/or migration. Antibodies which specifically bind DCL-1 or a fragment or derivative are particularly useful as therapeutic or diagnostic agents. Such antibodies may be monoclonal or polyclonal and may be selected from naturally occurring antibodies to DCL-1 or fragment or derivative thereof or may be specifically raised to DCL-1 or a fragment or derivative thereof. Alternatively the antibody may be a recombinant or synthetic antibody, including an antibody hybrid. Fragments of antibodies, such as Fab fragments, may also be used.

Both polyclonal and monoclonal antibodies are obtainable by immunization with DCL-1 or a fragment or derivative thereof. The methods of obtaining both types of sera are well known in the art, for example, the DCL-1 or fragment or derivative may first need to be associated with a carrier molecule. Polyclonal sera are typically prepared by injection of a suitable laboratory animal with an effective amount of DCL-1 or fragment or derivative thereof, or antigenic part thereof, collecting serum from the animal, and isolating specific sera by any of the known immunoadsorbent techniques.

Monoclonal antibodies may be produced in large quantities using hybridoma cell lines derived by fusing an immortal cell line and a lymphocyte sensitized against the immunogenic preparation. Such techniques are well known to those skilled in the art. (See, for example Douillard and Hoffman, Basic Facts about Hybridomas, in Compendium of Immunology Vol. II, ed. by Schwartz, 1981; Kohler and Milstein, Nature 256: 495-499, 1975; European Journal of Immunology 6: 511-519, 1976).

In addition, in some embodiments a nucleic acid molecule encoding DCL-1 or a biologically active fragment or derivative thereof, or a nucleic acid molecule encoding DCL-1 or a biologically active fragment or derivative thereof or a nucleic acid molecule antisense thereto, may be used to modulate the level and/or activity of DCL-1 or a biologically active fragment or derivative thereof and thereby modulate endocytosis, phagocytosis, cell adhesion and/or cell migration. The nucleic acid molecule may be a single or double stranded sequence of deoxyribonucleic acids such as cDNA sequences or a genomic sequence. A cDNA sequence may optionally comprise all or some of the 5′ or 3′ untranslated regions while a genomic sequence may also comprise introns. A genomic sequence may also include a promoter region or other regulatory regions. It should also be understood that the subject nucleic acid molecules may be a single or double stranded sequence of ribonucleic acids, such as mRNA.

The cDNA and genomic nucleotide sequences for human DCL-1 are provided by the sequence set forth in SEQ ID NOs: 7 and 9, respectively. Murine and rat cDNA DCL-1 sequence is provided by the nucleotide sequences set forth in SEQ ID NO: 11 and 14, respectively. SEQ ID NO: 18 discloses a partial sequence of bovine DCL-1.

Regarding nucleic acid molecules antisense to DCL-1, these will be DNA or RNA composed of the complementary sequence to DCL-1. Antisense nucleic acid molecules may be used, for example, in therapeutic strategies that use antisense DNA or RNA sequences to target specific gene DNA sequences or mRNA implicated in disease, in order to bind and physically inhibit their expression by physically blocking them. Nucleic acid molecules antisense to DCL-1 may hybridize to DCL-1 or a fragment thereof under high stringency conditions, which include and encompass from at least about 31% v/v to at least about 50% v/v formamide and from at least about 0.01M to at least about 0.15M salt for hybridization, and at least about 0.01M to at least about 0.15M salt for washing conditions. Stringency may be measured using a range of temperature such as from about 40° C. to about 65° C. Particularly useful stringency conditions are at 42° C. In general, washing is carried out at Tₘ=69.3+0.41 (G+C) %=−12° C. However, the Tₘ of a duplex DNA decreases by 1° C. with every increase of 1% in the number of mismatched based pairs (Bonner et al (1973) J. Mol. Biol., 81:123). Examples of such nucleic acid molecules antisense to DCL-1 or a fragment thereof are the nucleic acid molecules provided in SEQ ID NOs: 10, 13 and 17.

A nucleic acid molecule encoding DCL-1 or a biologically active fragment or derivative thereof may be ligated to a vector, such as an expression vector. Where the nucleic acid molecule has been ligated to an expression vector, the vector may be capable of expression in a prokaryotic cell (e.g. E. coli) or a eukaryotic cell (e.g. yeast cells, fungal cells, insect cells, mammalian cells or plant cells). The nucleic acid molecule may be ligated or fused or otherwise associated with a nucleic acid molecule encoding another entity such as, for example, a signal peptide. It may also comprise additional nucleotide sequence information fused, linked or otherwise associated with it either at the 3′ or 5′ terminal portions or at both the 3′ and 5′ terminal portions. The nucleic acid molecule may also be part of a vector, such as an expression vector. The latter embodiment facilitates production of recombinant forms of DCL-1 or a fragment or derivative thereof.

As mentioned above, DCL-1 of a biologically active fragment or derivative thereof, an antibody which specifically binds thereto, or a nucleic acid molecule which encodes DCL-1 or a fragment or derivative thereof or nucleic acid molecule antisense thereto may be used to modulate endocytosis, phagocytosis, cell adhesion and/or cell migration mediated by cells expressing DCL-1, including immune cells (e.g., antigen-presenting cells).

As used herein, “endocytosis” refers to a process by which materials enter a cell without passing through the cell membrane. Specifically, the cell membrane folds around the material outside the cell (“invagination”), resulting in the formation of a sac-like vesicle into which the material is incorporated. The vesicle is then pinched off from the cell surface so that it lies within the cell.

There are numerous methods of determining endocytosis. These include the use of ligands to study receptor-mediated endocytosis. This involves insertion of a receptor into the plasma membrane of a cell followed by endocytosis of the ligand-receptor complex. There are currently several products available for the study of receptor-mediated endocytosis, including conjugates of low-density lipoprotein, lipopolysaccharide, transferrin, EGF, hyaluronic acid and ovalbumin. Another method involves the use of labelled macromolecules and particles, including killed bacteria and yeast, dextrans and polystyrene microspheres, and liposomes.

“Phagocytosis” is a form of endocytosis where large particles are enveloped by the cell membrane of a phagocyte and internalized to form a phagosome, or food vacuole. “Phagocytes” are white blood cells, such as macrophages, monocytes, dendritic cells and neutrophils. For example, monocytes and macrophages are recruited to sites of inflammation, where they phagocytose pathogenic microbes and damaged tissue components and mediate local effector functions. Resident and recruited dendritic cells also phagocytose pathogens but after migrating into draining lymph nodes to present processed antigens to T and B lymphocytes to elicit antigen-specific adaptive immune responses.

“Cell adhesion” includes stimulating signals that regulate cell differentiation, the cell cycle, and cell survival. The adhesion of cells to each other or to the extracellular matrix is responsible for a wide range of normal and aberrant cellular activities, including migration of immune cells to sites of infection, invasion and metastasis of tumor cells, and angiogenesis during wound healing. To perform many of these functions, cells must bind other cells or various molecules in the extracellular matrix, such as to a DCL-1 ligand. Changes in cell adhesion can be the defining event of leukocyte involvement in a wide range of diseases, including cancer, osteoporosis, atherosclerosis, arthritis, infection, transplant reactions and inflammatory diseases.

Cell adhesion may be determined by a long-term assay or a conventional assay. Both of these involve seeding cells onto a substrate coated with one or more molecules of interest. Subsequently, adherent cells are fixed and stained. Relative attachment is determined using fluorescence and absorbance readings. Alternatively, cell adhesion may be determined in vitro and in vivo by direct cell-cell adhesion assays using line imaging. Still further, cell binding to fixed tissue sections may be assessed.

“Cell migration” refers to the movement of a population of cells from one place to another. In summary, cells of different origins migrate in an integrin dependent manner, involving (i) the formation of extensions at the cell front, (ii) integrin-dependent focal complex formation, (iii) maturation into and plasticity of focal contacts, and (iv) the controlled sliding and dispersal of focal contacts at the cell rear. Cell migration plays a central role in a wide variety of biological phenomena. For example, in embryogenesis, cellular migration is a recurring theme in morphogenic processes ranging from gastrulation to development of the nervous system. In an adult animal, cell migration is prominent in both physiological and pathological conditions. For example, migration of fibroblasts and vascular endothelial cells is essential for wound healing. In metastasis, tumor cells migrate from the initial tumor mass throughout the whole body.

There are several assays to determine cell migration. These include chemotaxis, haptotaxis, and cell invasion assays. Migration assays of these types may be conducted by several methods; the most commonly used being the Boyden Chamber assay. Haptotaxis migration assays measure cell movement toward an immobilized protein gradient and allow quantitative analysis. Chemotaxis assays assess the effects of compounds on the motility of a cell and analyze the migratory capacity of multiple cells types or lines in parallel. Invasion assays often involve the use of a membrane model, such as a basement membrane model, through which invasive cells are able to migrate. The invasive cells are then either stained or counted with a light microscope or detached, lysed and stained using fluorometric detection. Alternatively, genetically labelled cells may be tracked with fluorescent or other mature molecule readouts.

The modulation of endocytosis, phagocytosis, cell adhesion and/or cell migration may be used to treat and/or prevent diseases characterized by aberrant or unwanted endocytosis, phagocytosis, cell adhesion and/or cell migration mediated by cells expressing DCL-1, including immune cells (e.g., antigen-presenting cells).

The terms “modulate” or “modulated” mean changed or adjusted. Thus, the level of DCL-1 or a fragment or derivative thereof may be changed or adjusted. The level of DCL-1 or a fragment or derivative thereof may be increased or decreased. That is, the level of DCL-1 or a fragment or derivative thereof may be made greater or lesser. In some embodiments the level is modulated to that which would be expected to occur in a “normal” subject. A “normal” subject is one not experiencing a disease characterized by aberrant or unwanted endocytosis, phagocytosis, cell adhesion and/or cell migration.

Modulation of the level and/or activity of DCL-1 or a biologically active fragment or derivative thereof may be identified by any means known in the art. For example, identifying modulation of the level of DCL-1 can be achieved using techniques such as Western blotting, electrophoretic mobility shift assays and/or the readout of reporter genes. Alternatively, modulation of DCL-1 activity can be identified by screening for the modulation of endocytosis, phagocytosis, cell adhesion and/or cell migration. This is an example of an indirect system where modulation of DCL-1 expression per se is not the subject of identification.

As used herein, the term “aberrant” means differing from a level present in a subject not experiencing a disease characterized by aberrant or unwanted endocytosis, phagocytosis, cell adhesion and/or cell migration. The level of aberrant activity may be increased to decreased compared to a normal level. The term “unwanted” means not wanted or not needed and may, for example, be associated with an autoimmune disease. Reference to unwanted activity should be understood as a reference to overactivity, underactivity or to physiologically normal activity which is inappropriate in that it is unwanted.

Thus, the level of DCL-1 or a biologically active fragment or derivative thereof may be modulated to that present in a subject not experiencing a disease associated with not wanted or not needed endocytosis, phagocytosis, cell adhesion and/or cell migration.

Means for modulating endocytosis, phagocytosis, cell adhesion and/or cell migration would be well known to the person of skill in the art and include, but are not limited to:
(i) Introducing into a cell a nucleic acid encoding DCL-1 or a fragment or derivative thereof or nucleic acid molecule antisense thereto in order to modulate the capacity of said cell to express DCL-1 or the fragment or derivative thereof;
(ii) Introducing into a cell a proteinaceous or non-proteinaceous molecule which modulates transcriptional and/or translational regulation of a gene, wherein this gene may encode DCL-1 or a fragment or derivative thereof or some other gene which directly or indirectly modulates the expression of a nucleic acid molecule encoding DCL-1 or a fragment or derivative thereof;
(iii) Introducing a proteinaceous or non-proteinaceous molecule which functions as an antagonist of DCL-1 or the fragment or derivative thereof; and
(iv) Introducing a proteinaceous or non-proteinaceous molecule which functions as an agonist of DCL-1 or a fragment or derivative thereof (this should be understood to extend to administering the DCL-1 or fragment or derivative thereof).

For example, DCL-1 antisense sequences such as oligonucleotides may be introduced into a cell to down-regulate the expression and/or activity of DCL-1, and thereby down-regulate the endogenous level of DCL-1. Conversely, a nucleic acid molecule encoding DCL-1 or a fragment or derivative thereof may be introduced into a cell to enhance the level of expressed DCL-1 and/or activity by the cell.

The proteinaceous molecules described in points (i) to (iv), above, may be derived from any suitable source such as natural, recombinant or synthetic sources and include fusion proteins or molecules which have been identified following, for example, natural product screening. The reference to non-proteinaceous molecules may be, for example, a reference to a nucleic acid molecule or it may be a molecule derived from natural sources, such as for example natural product screening, or may be a chemically synthesized molecule. Alternatively, analogues of DCL-1 or a biologically active fragment or derivative thereof or small molecules capable of acting as agonists or antagonists may be used. Chemical agonists may not necessarily be derived from DCL-1 or a fragment or derivative thereof but may share certain conformational similarities. Alternatively, chemical agonists may be specifically designed to meet certain physiochemical properties. Antagonists may be any compound capable of blocking, inhibiting or otherwise preventing DCL-1 or a biologically active fragment or derivative thereof from carrying out its normal biological function. Antagonists include antibodies (e.g., monoclonal antibodies) that are immuno-interactive with DCL-1 and antisense nucleic acids which prevent transcription or translation of a DCL-1 gene or mRNA. Reference herein to “immuno-interactive” includes reference to any interaction, reaction, or other form of association between molecules and in particular where one of the molecules is, or mimics, a component of the immune system. Modulation of expression may also be achieved utilizing antigens, RNA, ribosomes, DNAzymes, RNA aptamers, antibodies or molecules suitable for use in cosuppression. The proteinaceous and non-proteinaceous molecules described herein are collectively referred to as “modulatory agents”.

The modulatory agents which are identified may take any suitable form. For example, proteinaceous agents may be glycosylated or unglycosylated, phosphorylated or dephosphorylated to various degrees and/or may contain a range of other molecules used, linked, bound or otherwise associated with the proteins such as amino acids, lipid, carbohydrates or other peptides, polypeptides or proteins. Similarly, the subject non-proteinaceous molecules may also take any suitable form. Both the proteinaceous and non-proteinaceous agents herein described may be linked, bound or otherwise associated with any other proteinaceous or non-proteinaceous molecules. For example, in one embodiment of the present invention, said agent is associated with a molecule which permits its targeting to a localized region.

The modulatory agent may act either directly or indirectly to modulate the level and/or activity DCL-1 or a biologically active fragment or derivative thereof. Said molecule acts directly if it associates with the DCL-1 nucleic acid molecule or expression product to modulate expression or activity, respectively. Said molecule acts indirectly if it associates with a molecule other than the DCL-1 nucleic acid molecule or expression product which other molecule either directly or indirectly modulates the expression or activity of the DCL-1 nucleic acid molecule or expression product, respectively. Accordingly, the method of the present invention encompasses the regulation of DCL-1 nucleic acid molecule expression or expression product activity via the induction of a cascade of regulatory steps

Accordingly, one embodiment provides a method for modulating endocytosis, phagocytosis, cell adhesion and/or cell migration mediated by a cell expressing DCL-1, especially an immune cell (e.g., an antigen-presenting cell), comprising administering DCL-1 or a fragment or derivative thereof or antibody which specifically binds thereto, or a nucleic acid molecule encoding DCL-1 or a fragment or derivative thereof or a nucleic acid molecule antisense thereto, to the cell for a time and under conditions sufficient to up-regulate, down-regulate or otherwise modulate the level and/or activity of DCL-1.

As used herein, the word “cell” refers to any type of cell irrespective of its origin, as DCL-1 mRNA is present in many tissues. For example, the cell may be a naturally occurring normal or abnormal cell or it may be manipulated, modified or otherwise treated either in vitro or in vivo such as a cell which has been freeze/thawed or genetically, biochemically or otherwise modified either in vitro or in vivo (including, for example, cells which are the result of the fusion of two distinct cell types). In some embodiments the cell is a leukocyte, such as a monocyte, macrophage, granulocyte or dendritic cell, including CD11⁺ (myeloid) and DC11⁻ (plasmacytoid) blood dendritic cells and monocyte-derived dendritic cells. It should be understood that the target cell which is treated according to the method of the present invention may be located ex vivo or in vivo

In some embodiments, the cell is an antigen-presenting cell, which includes professional or facultative antigen-presenting cells. Professional antigen-presenting cells function physiologically to present antigen in a form that is recognized by specific T cell receptors so as to stimulate or energies a T lymphocyte or B lymphocyte mediated immune response. Professional antigen-presenting cells not only process and present antigens in the context of the major histocompatability complex (MHC), but also possess the additional immunoregulatory molecules required to complete T cell activation or induce a tolerogenic response. Professional antigen-presenting cells include, but are not limited to, macrophages, monocytes, B lymphocytes, cells of myeloid lineage, including monocytic-granulocytic-DC precursors, marginal zone Kupffer cells, microglia, T cells, Langerhans cells and dendritic cells including interdigitating dendritic cells and follicular dendritic cells. Non-professional or facultative antigen-presenting cells typically lack one or more of the immunoregulatory molecules required to complete T lymphocyte activation or anergy. Examples of non-professional or facultative antigen-presenting cells include, but are not limited to, activated T lymphocytes, eosinophils, keratinocytes, astrocytes, follicular cells, microglial cells, thymic cortical cells, endothelial cells, Schwann cells, retinal pigment epithelial cells, myoblasts, vascular smooth muscle cells, chondrocytes, enterocytes, thymocytes, kidney tubule cells and fibroblasts. In some embodiments, the antigen-presenting cell is selected from monocytes, macrophages, cells of myeloid lineage, dendritic cells and Langerhans cells.

The term “expression” refers to the transcription and translation of a nucleic acid molecule. Reference to “expression product” is a reference to the product produced from the transcription and translation of a nucleic acid molecule. Increasing the expression of a nucleic acid molecule results in an increased level of the encoded protein. Conversely, decreasing the expression of a nucleic acid molecule results in a decreased level of the encoded protein.

Screening for the modulatory agents hereinbefore described can be achieved by any one of several suitable methods including, but in no way limited to, contacting a cell comprising a DCL-1 gene or fragment thereof with an agent and screening for the modulation of the level and/or activity of DCL-1 or a fragment or derivative thereof, modulation of the level of DCL-1 mRNA or a fragment thereof, and/or modulation of the level and/or activity of a downstream functional activity.

It should be understood that a nucleic acid molecule encoding DCL-1 or a biologically active fragment or derivative thereof used to screen for modulatory agents may be naturally occurring in the cell which is the subject of testing or it may have been transfected into a host cell for the purpose of testing. Further, the naturally occurring or transfected gene may be constitutively expressed—thereby providing a model useful for, inter alia, screening for agents which down regulate the level and/or activity of DCL-1 or a fragment or derivative thereof, or the gene may require activation—thereby providing a model useful for, inter alia, screening for agents which up regulate DCL-1 expression. Further, to the extent that a nucleic acid molecule encoding DCL-1 or a biologically active fragment or derivative thereof is transfected into a cell, that molecule may encode the entire DCL-1 gene or it may merely comprise a portion of the gene such as the portion which regulates expression of DCL-1. For example, the DCL-1 promoter region may be transfected into the cell which is the subject of testing. In this regard, where only the promoter is utilized, detecting modulation of the activity of the promoter can be achieved, for example, by ligating the promoter to a reporter gene. For example, the promoter may be ligated to luciferase or a CAT reporter, the modulation of expression of which gene can be detected via modulation of fluorescence intensity or CAT reporter activity, respectively.

These methods provide a mechanism for performing high throughput screening of putative modulatory agents such as the proteinaceous or non-proteinaceous agents comprising synthetic, combinatorial, chemical and natural libraries. These methods will also facilitate the detection of agents which bind either the DCL-1 nucleic acid molecule or expression product itself or which modulate the expression of an upstream molecule, which upstream molecule subsequently modulates DCL-1 expression or expression product activity. Accordingly, these methods provide a mechanism for detecting agents which either directly or indirectly modulate endocytosis, phagocytosis, cell adhesion and/or cell modulation. Thus, the level of modulation of endocytosis, phagocytosis, cell adhesion and/or cell migration may be used as surrogate markers of the level and/or activity of DCL-1 or a biologically active fragment or derivative thereof.

In order to modulate the level and/or activity of DCL-1 or a biologically active fragment or derivative thereof, the modulatory agent may be administered to the cell directly or indirectly. For example, the modulatory agent may be administered into the nucleus of the cell or may be administered to the medium surrounding the cell. The modulatory agent will be administered for a time and under conditions sufficient to modulate the level and/or activity or DCL-1 or a biologically active fragment or derivative thereof. As used herein, the phrase “for a time and under conditions sufficient” means any time and/or condition sufficient to modulate the level and/or activity of DCL-1 or a fragment or derivative thereof. Suitable time and conditions will depend upon the context under which the level and/or activity is to be modulated and will readily be determined by the skilled person.

DCL-1 or a biologically active fragment or derivative thereof, or antibody which specifically binds thereto, or a nucleic acid molecule encoding DCL-1 or a biologically active fragment or derivative thereof or nucleic acid molecule antisense thereto may be used in the treatment, prevention and or diagnosis of a disease associated with aberrant or unwanted endocytosis, phagocytosis, cell adhesion and/or cell migration mediated by cells expressing DCL-1, including immune cells (e.g., antigen-presenting cells).

Examples of diseases associated with aberrant or unwanted endocytosis, phagocytosis, cell adhesion and/or cell migration include those associated with hematopoiesis, leukocyte trafficking and/or phagocytic leukocyte immune effector functions, such as immunodeficiency diseases including severe combined immunodeficiency disease (SCID) and leukopenia, autoimmune diseases including diabetes and arthritis, and inflammatory diseases including arthritis, infectious diseases and diseases associated with transplant responses.

Hematopoiesis involves an interplay between the intrinsic genetic processes of blood cells and their environment. This interplay determines whether hematopoietic stem cells, progenitors, and mature blood cells remain quiescent, proliferate, differentiate, self-renew, or undergo apoptosis. Adherence of cells to microenvironmental elements can trigger a variety of signalling pathways, potentiate the responses to growth factors and modulate the downstream components of growth factor signalling cascades. Hematopoietic and nonhematopoietic cells that may regulate hematopoiesis include NK cells, T cells, macrophages, fibroblasts, osteoblasts, adipocytes, and perhaps even neurons. These cells may produce important growth factors, facilitate engraftment, or induce apoptosis.

Leukocyte trafficking is mediated by various cell adhesion molecules. Leukocyte trafficking between the blood and the tissues is pivotal for normal immune responses. Cell-adhesion molecules (such as selectins and leukocyte integrins) and chemoattractants (such as chemokines) have well-established roles in supporting leukocyte exit from the blood. These interactions are important for leukocyte extravasation and trafficking in all domestic animal species.

As mentioned above, phagocytic cells include macrophages and neutrophils. These cells act in the innate immune system by engulfing microorganisms, other cells, and foreign particles. Phagocytes distinguish healthy host cells from microbes and other host cells using receptors on their surface that recognize sugars present on microbes or sugars that are newly expressed on dead or damaged host cells. These sugars are not present on healthy host cells and therefore the host cells are not phagocytosed

Clearly, aberrant or unwanted hematopoiesis, leukocyte trafficking and phagocyte functions are involved in diseases such as cancer. Thus the inventors believe that DCL-1 or a biologically active fragment or derivative thereof (e.g., in the form of a soluble DCL-1 decoy), or antibody which specifically binds to DCL-1, or a nucleic acid molecule which encodes DCL-1 or a biologically active fragment thereof or nucleic acid molecule antisense thereto may be used to treat, prevent and/or diagnose diseases or conditions such as cancer, which are associated with aberrant or unwanted hematopoiesis, leukocyte trafficking and phagocyte functions. In particular, the inventors believe that modulating the level and/or activity of DCL-1, or a biologically active fragment or derivative thereof, may be used in the treatment and/or prevention of cancer metastasis because DCL-1 has been found to be expressed in macrophage podosomes and the podosomes of macrophages are similar to the inavdipodia of metastatic cells (Science 2006, 312:1868). Moreover, DCL-1 mRNA has been shown to be highly expressed in certain cell lines, including glioma cell lines (LN-18 and U-138), melanoma cell lines (SK-MEL-5 and M14), an adenocarcinoma cell line (SK-OV-3), a heptoma cell line (huh-7) and a renal cell carcinoma cell line (SN12C) (SymAtlas v1.2.4: on World Wide Web at symatlas.gnf.org/SymAtlas/, search for CD302).

The term “cancer” refers to any malignant growth or tumor caused by abnormal and uncontrolled cell division in any part of the body. These cells may invade other tissues, either by direct growth into adjacent tissue (invasion) or by migration to distant sites (metastasis). In some embodiments the cancer is glioma, melanoma, adenocarcinoma, heptoma, renal cell carcinoma, leukemia or lymphoma. In some embodiments the cancer is Hodgkin's Disease.

The subject of the treatment, prevention and/or diagnosis of a disease associated with aberrant or unwanted endocytosis, phagocytosis, cell adhesion and/or cell migration may be any subject in which DCL-1 or a fragment or derivative is present. For example, homologues of DCL-1 have been found in humans, mice and rats. Generally the subject will be a mammal such as, but not limited to, a human, primate, livestock animal (e.g. sheep, cow, horse, donkey, pig), companion animal (e.g. dog, cat), laboratory test animal (e.g. mouse, rabbit, rat, guinea pig, hamster), captive wild animal (e.g. fox, deer).

The disease may be treated and/or prevented by administering to the subject an effective amount of a modulatory agent as hereinbefore described for a time and under conditions sufficient to modulate the level and/or activity of DCL-1 or a biologically active fragment or derivative thereof. An “effective amount” means an amount necessary at least partly to attain the desired immune response, or to delay the onset or inhibit progression or halt altogether, the onset or progression of a particular condition being treated. The amount varies depending upon the health and physical condition of the individual to be treated, the taxonomic group of the subject to be treated, the degree of protection desired, the formulation of the composition, the assessment of the medical situation, and other relevant factors. It is expected that the amount will fall in a relatively broad range that can be determined through routine trials.

Reference herein to “treatment” and “prevention” is to be considered in its broadest context. The term “treatment” does not necessarily imply that a subject is treated until total recovery. Therefore, the treatment need not achieve a complete “cure”, or eradicate every symptom or manifestation of a disease, to constitute a viable therapy. As is recognized in the pertinent field, a treatment may reduce the severity of a given disease state, but need not abolish every manifestation of the disease to be regarded as a useful treatment. Similarly, “prevention” does not necessarily mean that the subject will not eventually contract a disease condition. Accordingly, treatment and prevention include amelioration of the symptoms of a particular condition or preventing or otherwise reducing the risk of developing a particular condition. The term “prevention” may be considered as reducing the severity or onset of a particular condition. “Treatment” may also reduce the severity of an existing condition.

The modulatory agent may be administered in a form that has been bound to or chemically coupled or linked to one or more antigens. It is believed that the modulatory agent will, for example, modulate trafficking to peripheral lymph nodes for antigen presentation to T and B cells located therein. Thus, the modulatory agent may facilitate binding and co-stimulation of T cells. In other embodiments the modulatory agent may be used to alter the pattern of antigen presenting cell trafficking to specific organs of choice, such as preferentially trafficking to draining lymph nodes, spleen and the like.

In some embodiments, the modulatory agent is a DCL-1 agonist, which is suitably selected from an anti-DCL-1 antibody or fragment thereof, a DCL-1 natural ligand or derivative thereof, or an anti-idiotypic antibody directed against an anti-natural ligand antibody. In illustrative examples of this type, the DCL-1 agonist is coupled, linked or conjugated to an antigen associated with a condition or disease.

Thus, where the disease is an infectious disease, the modulatory agent may be administered in concert with one or more antigens of an infectious agent (e.g., a pathogenic organism) to upregulate endocytosis, phagocytosis, cell adhesion, and/or cell migration and thereby elicit a targeted immune response against the infectious agent in the subject and consequent treatment of the disease. Alternatively, where the disease is a cancer, the modulatory agent may be administered in concert with a cancer or tumor antigen to upregulate endocytosis, phagocytosis, cell adhesion, and/or cell migration and thereby elicit a targeted immune response against the cancerous cells in the subject and consequent treatment of the cancer. In another example, where the disease is associated with an unwanted or deleterious immune response such as in an autoimmune disease or in a transplantation associated disease, the modulatory agent may be co-administered with a self or tumor antigen to upregulate endocytosis, phagocytosis, cell adhesion, and/or cell migration and thereby elicit a targeted tolerogenic immune response in the subject and consequent treatment of the unwanted or deleterious immune response. In these embodiments, the present invention contemplates any antigen that corresponds to at least a portion of a target antigen of interest for modulating an immune response to that target antigen. Such an antigen may be in soluble form (e.g., peptide or polypeptide) or in the form of whole cells or attenuated pathogen preparations (e.g., attenuated virus or bacteria) or it may be presented by antigen-presenting cells.

Target antigens useful in the present invention can be any type of biological molecule including, for example, simple intermediary metabolites, sugars, lipids, and hormones as well as macromolecules such as complex carbohydrates, phospholipids, nucleic acids, polypeptides and peptides. Target antigens may be selected from endogenous antigens produced by a host or exogenous antigens that are foreign to the host. Suitable endogenous antigens include, but are not restricted to, self-antigens that are targets of autoimmune responses as well as cancer or tumor antigens. Illustrative examples of self antigens useful in the treatment or prevention of autoimmune disorders include, but not limited to, diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjögren's Syndrome, including keratoconjunctivitis sicca secondary to Sjögren's Syndrome, alopecia areata, allergic responses due to arthropod bite reactions, Crohn's disease, ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves opthalmopathy, sarcoidosis, primary biliary cirrhosis, uveitis posterior, and interstitial lung fibrosis. Other autoantigens include those derived from nucleosomes for the treatment of systemic lupus erythematosus (e.g., GenBank Accession No. D28394; Bruggen et al., 1996, Ann. Med. Interne (Paris), 147:485-489) and from the 44,000 Da peptide component of ocular tissue cross-reactive with O. volvulus antigen (McKeclmie et al., 1993, Ann Trop. Med. Parasitol. 87:649-652). Thus, illustrative autoantigens antigens that can be used in the compositions and methods of the present invention include, but are not limited to, at least a portion of a lupus autoantigen, Smith, Ro, La, U1-RNP, fibrillin (scleroderma), pancreatic β cell antigens, GAD65 (diabetes related), insulin, myelin basic protein, myelin proteolipid protein, histones, PLP, collagen, glucose-6-phosphate isomerase, citrullinated proteins and peptides, thyroid antigens, thyroglobulin, thyroid-stimulating hormone (TSH) receptor, various tRNA synthetases, components of the acetyl choline receptor (AchR), MOG, proteinase-3, myeloperoxidase, epidermal cadherin, acetyl choline receptor, platelet antigens, nucleic acids, nucleic acid:protein complexes, joint antigens, antigens of the nervous system, salivary gland proteins, skin antigens, kidney antigens, heart antigens, lung antigens, eye antigens, erythrocyte antigens, liver antigens and stomach antigens.

Non-limiting examples of cancer or tumor antigens include antigens from a cancer or tumor selected from ABL1 protooncogene, AIDS related cancers, acoustic neuroma, acute lymphocytic leukemia, acute myeloid leukemia, adenocystic carcinoma, adrenocortical cancer, agnogenic myeloid metaplasia, alopecia, alveolar soft-part sarcoma, anal cancer, angiosarcoma, aplastic anemia, astrocytoma, ataxia-telangiectasia, basal cell carcinoma (skin), bladder cancer, bone cancers, bowel cancer, brain stem glioma, brain and CNS tumors, breast cancer, CNS tumors, carcinoid tumors, cervical cancer, childhood brain tumors, childhood cancer, childhood leukemia, childhood soft tissue sarcoma, chondrosarcoma, choriocarcinoma, chronic lymphocytic leukemia, chronic myeloid leukemia, colorectal cancers, cutaneous t-cell lymphoma, dermatofibrosarcoma-protuberans, desmoplastic-small-round-cell-tumor, ductal carcinoma, endocrine cancers, endometrial cancer, ependymoma, oesophageal cancer, Ewing's Sarcoma, Extra-Hepatic Bile Duct Cancer, Eye Cancer, Eye: Melanoma, Retinoblastoma, Fallopian Tube cancer, Fanconi anemia, fibrosarcoma, gall bladder cancer, gastric cancer, gastrointestinal cancers, gastrointestinal-carcinoid-tumor, genitourinary cancers, germ cell tumors, gestational-trophoblastic-disease, glioma, gynecological cancers, hematological malignancies, hairy cell leukemia, head and neck cancer, hepatocellular cancer, hereditary breast cancer, histiocytosis, Hodgkin's disease, human papillomavirus, hydatidiform mole, hypercalcemia, hypopharynx cancer, intraocular melanoma, islet cell cancer, Kaposi's sarcoma, kidney cancer, Langerhan's-cell-histiocytosis, laryngeal cancer, leiomyosarcoma, leukemia, Li-Fraumeni syndrome, lip cancer, liposarcoma, liver cancer, lung cancer, lymphedema, lymphoma, Hodgkin's lymphoma, non-Hodgkin's lymphoma, male breast cancer, malignant-rhabdoid-tumor-of-kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, nasal cancer, nasopharyngeal cancer, nephroblastoma, neuroblastoma, neurofibromatosis, Nijmegen breakage syndrome, non-melanoma skin cancer, non-small-cell-lung-cancer (NSCLC), ocular cancers, oesophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal-tumors, pituitary cancer, polycythemia vera, prostate cancer, rare-cancers-and-associated-disorders, renal cell carcinoma, retinoblastoma, rhabdomyosarcoma, Rothmund-Thomson syndrome, salivary gland cancer, sarcoma, schwannoma, Sezary syndrome, skin cancer, small cell lung cancer (SCLC), small intestine cancer, soft tissue sarcoma, spinal cord tumors, squamous-cell-carcinoma-(skin), stomach cancer, synovial sarcoma, testicular cancer, thymus cancer, thyroid cancer, transitional-cell-cancer-(bladder), transitional-cell-cancer-(renal-pelvis-/-ureter), trophoblastic cancer, urethral cancer, urinary system cancer, uroplakins, uterine sarcoma, uterus cancer, vaginal cancer, vulva cancer, Waldenstrom's-Macroglobulinemia, Wilms' Tumor. In certain embodiments, the cancer or tumor relates to melanoma. Illustrative examples of melanoma-related antigens include melanocyte differentiation antigen (e.g., gp100, MART, Melan-A/MART-1, TRP-1, Tyros, TRP2, MC1R, MUC1F, MUC1R or a combination thereof) and melanoma-specific antigens (e.g., BAGE, GAGE-1, gp100In4, MAGE-1 (e.g., GenBank Accession No. X54156 and AA494311), MAGE-3, MAGE4, PRAME, TRP2IN2, NYNSO1a, NYNSO1b, LAGE1, p97 melanoma antigen (e.g., GenBank Accession No. M12154) p5 protein, gp75, oncofetal antigen, GM2 and GD2 gangliosides, cdc27, p21ras, gp100^{Pmel117} or a combination thereof. Other tumor-specific antigens include, but are not limited to: etv6, aml1, cyclophilin b (acute lymphoblastic leukemia); Ig-idiotype (B cell lymphoma); E-cadherin, α-catenin, β-catenin, γ-catenin, p120ctn (glioma); p21 ras (bladder cancer); p21 ras (biliary cancer); MUC family, HER2/neu, c-erbB-2 (breast cancer); p53, p21ras (cervical carcinoma); p21ras, HER2/neu, c-erbB-2, MUC family, Cripto-1 protein, Pim-1 protein (colon carcinoma); Colorectal associated antigen (CRC)-CO17-1A/GA733, APC (colorectal cancer); carcinoembryonic antigen (CEA) (colorectal cancer; choriocarcinoma); cyclophilin b (epithelial cell cancer); HER2/neu, c-erbB-2, ga733 glycoprotein (gastric cancer); α-fetoprotein (hepatocellular cancer); Imp-1, EBNA-1 (Hodgkin's lymphoma); CEA, MAGE-3, NY-ESO-1 (lung cancer); cyclophilin b (lymphoid cell-derived leukemia); MUC family, p21ras (myeloma); HER2/neu, c-erbB-2 (non-small cell lung carcinoma); Imp-1, EBNA-1 (nasopharyngeal cancer); MUC family, HER2/neu, c-erbB-2, MAGE-A4, NY-ESO-1 (ovarian cancer); Prostate Specific Antigen (PSA) and its antigenic epitopes PSA-1, PSA-2, and PSA-3, PSMA, HER2/neu, c-erbB-2, ga733 glycoprotein (prostate cancer); HER2/neu, c-erbB-2 (renal cancer); viral products such as human papilloma virus proteins (squamous cell cancers of the cervix and esophagus); NY-ESO-1 (testicular cancer); and HTLV-1 epitopes (T cell leukemia).

Foreign antigens are suitably selected from transplantation antigens, allergens as well as antigens from pathogenic organisms. Transplantation antigens can be derived from donor cells or tissues from e.g., heart, lung, liver, pancreas, kidney, neural graft components, or from the donor antigen-presenting cells bearing MHC loaded with self antigen in the absence of exogenous antigen.

Non-limiting examples of allergens include Fel d 1 (i.e., the feline skin and salivary gland allergen of the domestic cat Felis domesticus, the amino acid sequence of which is disclosed International Publication WO 91/06571), Der p I, Der p II, Der fl or Der fII (i.e., the major protein allergens from the house dust mite dermatophagoides, the amino acid sequence of which is disclosed in International Publication WO 94/24281). Other allergens may be derived, for example from the following: grass, tree and weed (including ragweed) pollens; fungi and moulds; foods such as fish, shellfish, crab, lobster, peanuts, nuts, wheat gluten, eggs and milk; stinging insects such as bee, wasp, and hornet and the chirnomidae (non-biting midges); other insects such as the housefly, fruit fly, sheep blow fly, screw worm fly, grain weevil, silkworm, honeybee, non-biting midge larvae, bee moth larvae, mealworm, cockroach and larvae of Tenibrio molitor beetle; spiders and mites, including the house dust mite; allergens found in the dander, urine, saliva, blood or other bodily fluid of mammals such as cat, dog, cow, pig, sheep, horse, rabbit, rat, guinea pig, mouse and gerbil; airborne particulates in general; latex; and protein detergent additives.

Exemplary pathogenic organisms include, but are not limited to, viruses, bacteria, fungi parasites, algae and protozoa and amoebae. Illustrative examples of viruses include viruses responsible for diseases including, but not limited to, measles, mumps, rubella, poliomyelitis, hepatitis A, B (e.g., GenBank Accession No. E02707), and C (e.g., GenBank Accession No. E06890), as well as other hepatitis viruses, influenza, adenovirus (e.g., types 4 and 7), rabies (e.g., GenBank Accession No. M34678), yellow fever, Epstein-Barr virus and other herpes viruses such as papillomavirus, Ebola virus, influenza virus, Japanese encephalitis (e.g., GenBank Accession No. E07883), dengue (e.g., GenBank Accession No. M24444), hantavirus, Sendai virus, respiratory syncytial virus, othromyxoviruses, vesicular stomatitis virus, visna virus, cytomegalovirus and human immunodeficiency virus (HIV) (e.g., GenBank Accession No. U18552). Any suitable antigen derived from such viruses are useful in the practice of the present invention. For example, illustrative retroviral antigens derived from HIV include, but are not limited to, antigens such as gene products of the gag, pol, and env genes, the Nef protein, reverse transcriptase, and other HIV components. Illustrative examples of hepatitis viral antigens include, but are not limited to, antigens such as the S, M, and L proteins of hepatitis B virus, the pre-S antigen of hepatitis B virus, and other hepatitis, e.g., hepatitis A, B, and C, viral components such as hepatitis C viral RNA. Illustrative examples of influenza viral antigens include; but are not limited to, antigens such as hemagglutinin and neuraminidase and other influenza viral components. Illustrative examples of measles viral antigens include, but are not limited to, antigens such as the measles virus fusion protein and other measles virus components. Illustrative examples of rubella viral antigens include, but are not limited to, antigens such as proteins E1 and E2 and other rubella virus components; rotaviral antigens such as VP7sc and other rotaviral components. Illustrative examples of cytomegaloviral antigens include, but are not limited to, antigens such as envelope glycoprotein B and other cytomegaloviral antigen components. Non-limiting examples of respiratory syncytial viral antigens include antigens such as the RSV fusion protein, the M2 protein and other respiratory syncytial viral antigen components. Illustrative examples of herpes simplex viral antigens include, but are not limited to, antigens such as immediate early proteins, glycoprotein D, and other herpes simplex viral antigen components. Non-limiting examples of varicella zoster viral antigens include antigens such as 9PI, gpII, and other varicella zoster viral antigen components. Non-limiting examples of Japanese encephalitis viral antigens include antigens such as proteins E, M-E, M-E-NS1, NS 1, NS 1-NS2A, 80% E, and other Japanese encephalitis viral antigen components. Representative examples of rabies viral antigens include, but are not limited to, antigens such as rabies glycoprotein, rabies nucleoprotein and other rabies viral antigen components. Illustrative examples of papillomavirus antigens include, but are not limited to, the L1 and L2 capsid proteins as well as the E6/E7 antigens associated with cervical cancers, See Fundamental Virology, Second Edition, eds. Fields, B. N. and Knipe, D. M., 1991, Raven Press, New York, for additional examples of viral antigens.

Illustrative examples of fungi include Acremonium spp., Aspergillus spp., Basidiobolus spp., Bipolaris spp., Blastomyces dermatidis, Candida spp., Cladophialophora carrionii, Coccoidiodes immitis, Conidiobolus spp., Cryptococcus spp., Curvularia spp., Epidermophyton spp., Exophiala jeanselmei, Exserohilum spp., Fonsecaea compacta, Fonsecaea pedrosoi, Fusarium oxysporum, Fusarium solani, Geotrichum candidum, Histoplasma capsulatum var. capsulatum, Histoplasma capsulatum var. duboisii, Hortaea werneckii, Lacazia loboi, Lasiodiplodia theobromae, Leptosphaeria senegalensis, Madurella grisea, Madurella mycetomatis, Malassezia furfur, Microsporum spp., Neotestudina rosatii, Onychocola canadensis, Paracoccidioides brasiliensis, Phialophora verrucosa, Piedraia hortae, Piedra iahortae, Pityriasis versicolor, Pseudallesheria boydii, Pyrenochaeta romeroi, Rhizopus arrhizus, Scopulariopsis brevicaulis, Scytalidium dimidiatum, Sporothrix schenckii, Trichophyton spp., Trichosporon spp., Zygomcete fungi, Absidia corymbifera, Rhizomucor pusillus and Rhizopus arrhizus. Thus, illustrative fungal antigens that can be used in the compositions and methods of the present invention include, but are not limited to, candida fungal antigen components; histoplasma fungal antigens such as heat shock protein 60 (HSP60) and other histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components.

Illustrative examples of bacteria include bacteria that are responsible for diseases including, but not restricted to, diphtheria (e.g., Corynebacterium diphtheria), pertussis (e.g., Bordetella pertussis, GenBank Accession No. M35274), tetanus (e.g., Clostridium tetani, GenBank Accession No. M64353), tuberculosis (e.g., Mycobacterium tuberculosis), bacterial pneumonias (e.g., Haemophilus influenzae), cholera (e.g., Vibrio cholerae), anthrax (e.g., Bacillus anthracis), typhoid, plague, shigellosis (e.g., Shigella dysenteriae), botulism (e.g., Clostridium botulinum), salmonellosis (e.g., GenBank Accession No. L03833), peptic ulcers (e.g., Helicobacter pylori), Legionnaire's Disease, Lyme disease (e.g., GenBank Accession No. U59487). Other pathogenic bacteria include Escherichia coli, Clostridium perfringens, Pseudomonas aeruginosa, Staphylococcus aureus and Streptococcus pyogenes. Thus, bacterial antigens which can be used in the compositions and methods of the invention include, but are not limited to: pertussis bacterial antigens such as pertussis toxin, filamentous hemagglutinin, pertactin, F M2, FIM3, adenylate cyclase and other pertussis bacterial antigen components; diphtheria bacterial antigens such as diphtheria toxin or toxoid and other diphtheria bacterial antigen components; tetanus bacterial antigens such as tetanus toxin or toxoid and other tetanus bacterial antigen components, streptococcal bacterial antigens such as M proteins and other streptococcal bacterial antigen components; gram-negative bacilli bacterial antigens such as lipopolysaccharides and other gram-negative bacterial antigen components; Mycobacterium tuberculosis bacterial antigens such as mycolic acid, heat shock protein 65 (HSP65), the 30 kDa major secreted protein, antigen 85A and other mycobacterial antigen components; Helicobacter pylori bacterial antigen components, pneumococcal bacterial antigens such as pneumolysin, pneumococcal capsular polysaccharides and other pneumococcal bacterial antigen components; Haemophilus influenza bacterial antigens such as capsular polysaccharides and other Haemophilus influenza bacterial antigen components; anthrax bacterial antigens such as anthrax protective antigen and other anthrax bacterial antigen components; rickettsiae bacterial antigens such as rompA and other rickettsiae bacterial antigen component. Also included with the bacterial antigens described herein are any other bacterial, mycobacterial, mycoplasmal, rickettsial, or chlamydial antigens.

Illustrative examples of protozoa include protozoa that are responsible for diseases including, but not limited to, malaria (e.g., GenBank Accession No. X53832), hookworm, onchocerciasis (e.g., GenBank Accession No. M27807), schistosomiasis (e.g., GenBank Accession No. LOS198), toxoplasmosis, trypanosomiasis, leishmaniasis, giardiasis (GenBank Accession No. M33641), amoebiasis, filariasis (e.g., GenBank Accession No. J03266), borreliosis, and trichinosis. Thus, protozoal antigens which can be used in the compositions and methods of the invention include, but are not limited to: plasmodium falciparum antigens such as merozoite surface antigens, sporozoite surface antigens, circumsporozoite antigens, gametocyte/gamete surface antigens, blood-stage antigen pf 155/RESA and other plasmodial antigen components; toxoplasma antigens such as SAG-1, p30 and other toxoplasmal antigen components; schistosomae antigens such as glutathione-S-transferase, paramyosin, and other schistosomal antigen components; leishmania major and other leishmaniae antigens such as gp63, lipophosphoglycan and its associated protein and other leishmanial antigen components; and trypanosoma cruzi antigens such as the 75-77 kDa antigen, the 56 kDa antigen and other trypanosomal antigen components.

The present invention also contemplates toxin components as antigens. Illustrative examples of toxins include, but are not restricted to, staphylococcal enterotoxins, toxic shock syndrome toxin; retroviral antigens (e.g., antigens derived from HIV), streptococcal antigens, staphylococcal enterotoxin-A (SEA), staphylococcal enterotoxin-B (SEB), staphylococcal enterotoxin₁₋₃ (SE₁₋₃), staphylococcal enterotoxin-D (SED), staphylococcal enterotoxin-E (SEE) as well as toxins derived from mycoplasma, mycobacterium, and herpes viruses.

The invention also contemplates modifying peptide antigens using ordinary molecular biological techniques so as to alter their resistance to proteolytic degradation or to optimize solubility properties or to render them more suitable as an immunogenic agent.

Peptide antigens may be of any suitable size that can be utilized to stimulate or inhibit an immune response to a target antigen of interest. A number of factors can influence the choice of peptide size. For example, the size of a peptide can be chosen such that it includes, or corresponds to the size of, T cell epitopes and/or B cell epitopes, and their processing requirements. Practitioners in the art will recognize that class I-restricted T cell epitopes are typically between 8 and 10 amino acid residues in length and if placed next to unnatural flanking residues, such epitopes can generally require 2 to 3 natural flanking amino acid residues to ensure that they are efficiently processed and presented. Class II-restricted T cell epitopes usually range between 12 and 25 amino acid residues in length and may not require natural flanking residues for efficient proteolytic processing although it is believed that natural flanking residues may play a role. Another important feature of class II-restricted epitopes is that they generally contain a core of 9-10 amino acid residues in the middle which bind specifically to class II MHC molecules with flanking sequences either side of this core stabilizing binding by associating with conserved structures on either side of class II MHC antigens in a sequence independent manner. Thus the functional region of class II-restricted epitopes is typically less than about 15 amino acid residues long. The size of linear B cell epitopes and the factors effecting their processing, like class II-restricted epitopes, are quite variable although such epitopes are frequently smaller in size than 15 amino acid residues. From the foregoing, it is advantageous, but not essential, that the size of the peptide is at least 6, 7, 8, 9, 10, 11, 12, 13, 14, 15, 20, 25, 30 amino acid residues. Suitably, the size of the peptide is no more than about 500, 200, 100, 80, 60, 50, 40 amino acid residues. In certain advantageous embodiments, the size of the peptide is sufficient for presentation by an antigen-presenting cell of a T cell and/or a B cell epitope contained within the peptide.

Criteria for identifying and selecting effective antigenic peptides (e.g., minimal peptide sequences capable of eliciting an immune response) can be found in the art. For example, Apostolopoulos-et al. (2000, Curr. Opin. Mol. Ther. 2:29-36) discusses the strategy for identifying minimal antigenic peptide sequences based on an understanding of the three dimensional structure of an antigen-presenting molecule and its interaction with both an antigenic peptide and T-cell receptor. Shastri (1996, Curr. Opin. Immunol. 8:271-277) discloses how to distinguish rare peptides that serve to activate T cells from the thousands peptides normally bound to MHC molecules.

Administration of the modulatory agent is typically in the form of a pharmaceutical composition and may be by any convenient means, depending on the particular case. The variation depends, for example, on the human or animal and the modulatory agent chosen. A broad range of doses may be applicable. Considering a human subject, for example, from about 0.1 mg to about 1 mg of modulatory agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

The modulatory agent may be administered in a convenient manner such as by the oral, intravenous (where water soluble), intraperitoneal, intramuscular, subcutaneous, intradermal or suppository routes or implanting (e.g. using slow release molecules). The modulatory agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

In addition, the modulatory agent may be coadministered or sequentially administered with one or more other compounds or molecules. By “coadministered” is meant simultaneous administration in the same formulation or in two different formulations via the same or different routes or sequential administration by the same or different routes. By “sequential” administration is meant a time difference of from seconds, minutes, hours or days between the administration of the two types of molecules. These molecules may be administered in any order.

The modulatory agent may be administered in the form of a pharmaceutical composition, comprising a modulatory agent as hereinbefore defined and one or more pharmaceutically acceptable carriers and/or diluents. Said modulatory agents are referred to as the active ingredients.

The pharmaceutical compositions suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The preventions of the action of microorganisms can be brought about by various antibacterial and antifungal agents, for example, parabens, chlorobutanol, phenol, sorbic acid, thimerosal and the like. In many cases isotonic agents, for example, sugars or sodium chloride may be used. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavoring agent such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the active compound(s) may be incorporated into sustained-release preparations and formulations.

The pharmaceutical composition may also comprise genetic molecules such as a vector capable of transfecting target cells where the vector carries a nucleic acid molecule encoding a modulatory agent. The vector may, for example, be a viral vector.

Yet another embodiment provides a method of diagnosing in a subject a disease associated with aberrant or unwanted endocytosis, phagocytosis, cell adhesion and/or cell migration, comprising identifying the level and/or activity of DCL-1 or a fragment or derivative thereof in a biological sample isolated from the subject. For example, screening for the levels of DCL-1 protein or DCL-1 mRNA transcripts in tissues may be used as an indicator of a predisposition to, or the development of a disease associated with aberrant or unwanted endocytosis, phagocytosis, cell adhesion and/or cell migration that is mediated by cells expressing DCL-1. More specifically, there is now provided a means for screening subjects for the presence of DCL-1 or a fragment or derivative thereof or a nucleic acid which encodes DCL-1 or a fragment or derivative thereof which are transcribed and/or translated by a given population of cells. The screening methodology may be directed to qualitative and/or quantitative DCL-1 analysis.

Screening for DCL-1 or a fragment or derivative thereof or a nucleic acid which encodes DCL-1 or a fragment or derivative thereof in a biological sample can be performed by any one of a number of suitable methods which are well known to those skilled in the art. Examples of suitable methods include, but are not limited to, in situ hybridization of biopsy sections to detect mRNA transcript or DNA, Northern blotting, RT-PCR of specimens isolated from tissue biopsies or antibody screening of tissue sections.

To the extent that antibody based methods of diagnosis are used, the presence of DCL-1 or a fragment or derivative thereof may be determined in a number of ways such as by Western blotting, ELISA or flow cytometry procedures. These, of course, include both single-site and two-site or “sandwich” assays of the non-competitive types, as well as in the traditional competitive binding assays. These assays also include direct binding of a labelled antibody to a target.

Sandwich assays are among the most useful and commonly used assays and are favored for use in the present invention. A number of variations of the sandwich assay technique exist. Briefly, in a typical forward assay, an unlabelled antibody is immobilized on a solid substrate and the sample to be tested brought into contact with the bound molecule. After a suitable period of incubation, for a period of time sufficient to allow formation of an antibody-antigen complex, a second antibody specific to the antigen, labelled with a reporter molecule capable of producing a detectable signal is then added and incubated, allowing time sufficient for the formation of another complex of antibody-antigen-labelled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of hapten. Variations on the forward assay include a simultaneous assay, in which both sample and labelled antibody are added simultaneously to the bound antibody. These techniques are well known to those skilled in the art, including any minor variations as will be readily apparent. The sample may contain DCL-1 or a fragment or derivative thereof or DCL-1 or a fragment thereof, including cell extract, tissue biopsy or possibly serum, saliva, mucosal secretions, lymph, tissue fluid and respiratory fluid. The sample is, therefore, generally a biological sample comprising biological fluid but also extends to fermentation fluid and supernatant fluid such as from a cell culture.

In the typical forward sandwich assay, a first antibody having specificity for DCL-1 or an antigenic part thereof, is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes) and under suitable conditions (e.g. 25° C.) to allow binding of any subunit present in the antibody. Following the incubation period, the antibody subunit solid phase is washed and dried and incubated with a second antibody specific for a portion of the hapten. The second antibody is linked to a reporter molecule which is used to indicate the binding of the second antibody to the hapten.

An alternative method involves immobilizing the target molecules in the biological sample and then exposing the immobilized target to specific antibody which may or may not be labelled with a reporter molecule. Depending on the amount of target and the strength of the reporter molecule signal, a bound target may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

By “reporter molecule” as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody hapten complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of hapten which was present in the sample. “Reporter molecule” also extends to use of cell agglutination or inhibition of agglutination such as red blood cells on latex beads, and the like.

Alternately, fluorescent compounds, such as fluorescein and rhodamine, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic color visually detectable with a light microscope. As in the EIA, the fluorescent labelled antibody is allowed to bind to the first antibody-hapten complex. After washing off the unbound reagent, the remaining tertiary complex is then exposed to the light of the appropriate wavelength the fluorescence observed indicates the presence of the hapten of interest. Immunofluorescent and EIA techniques are both very well established in the art and are particularly preferred for the present method. However, other reporter molecules, such as radioisotope, chemiluminescent or bioluminescent molecules, may also be employed.

TABLE 1

| SEQ ID NO | SEQUENCE DESCRIPTION |
|---|---|
| <400>1 | Human DEC205/DCL-1 splice variant (exon 34 fusion): |
| | cDNA sequence |
| <400>2 | Human DEC205/DCL-1 splice variant (exon 34 fusion): |
| | amino acid sequence |
| <400>3 | Human DEC205/DCL-1 splice variant (exon 34 fusion): |
| | complementary DNA strand |
| <400>4 (20) | Human DEC-205/DCL-1 cDNA (exon 33 fusion) sequence |
| <400>5 (21) | Human DEC-205/DCL-1 amino acid (exon 33 fusion) |
| | sequence |
| <400>6 (22) | Human DEC-205/DCL-1 (exon 33 fusion) complementary |
| | DNA strand sequence |
| <400>7 | Human DCL-1 cDNA sequence |
| <400>8 | Human DCL-1 amino acid sequence |
| <400>9 | Human DCL-1 genomic DNA sequence |
| <400>10 | Human DCL-1 complementary DNA sequence |
| <400>11 | Murine DCL-1 cDNA sequence |
| <400>12 | Murine DCL-1 amino acid sequence |
| <400>13 | Murine DCL-1 complementary DNA sequence |
| <400>14 | Rat DCL-1 cDNA sequence |
| <400>15 | Rat DCL-1 partial amino acid sequence |
| <400> 16 | Rat DCL-1 full amino acid sequence |
| <400>17 | Rat DCL-1 complementary DNA sequence |
| <400>18 | Bovine DCL-1 EST sequence |
| <400>19 | Primer MK062 |
| <400>20 | Primer MK0636 |
| <400>21 | Primer MK333 |
| <400>22 | Primer MK211 |
| <400>23 | Human DCL-1 ortholog |
| <400> 24 | Rat DCL-1 ortholog 1 |
| <400> 25 | Rat DCL-1 ortholog 2 |
| <400> 26 | Mouse DCL-1 ortholog 1 |
| <400> 27 | Mouse DCL-1 ortholog 2 |
| <400>28 | C-type lectin domain sequence of human DCSIGN/CD209 |
| <400>29 | C-type lectin domain sequence of human MGL/CD301 |
| <400>30 | C-type lectin domain sequence of human MMR/CD206 |
| | CRD4 |
| <400>31 | C-type lectin domain sequence of human hDCL-1 |

Further features of the present invention are more fully described in the following non-limiting examples.

### EXAMPLES

### Example 1

### Characterisation of DCL-1

As mentioned above, DCL-1 is a type I transmembrane C-type lectin receptor. It is also known as CD302. DCL-1 is the simplest type I transmembrane C-type lectin discovered so far: containing a SP, one C-type lectin-like domain (CTLD) and one short spacer, followed by one TM and one CP.

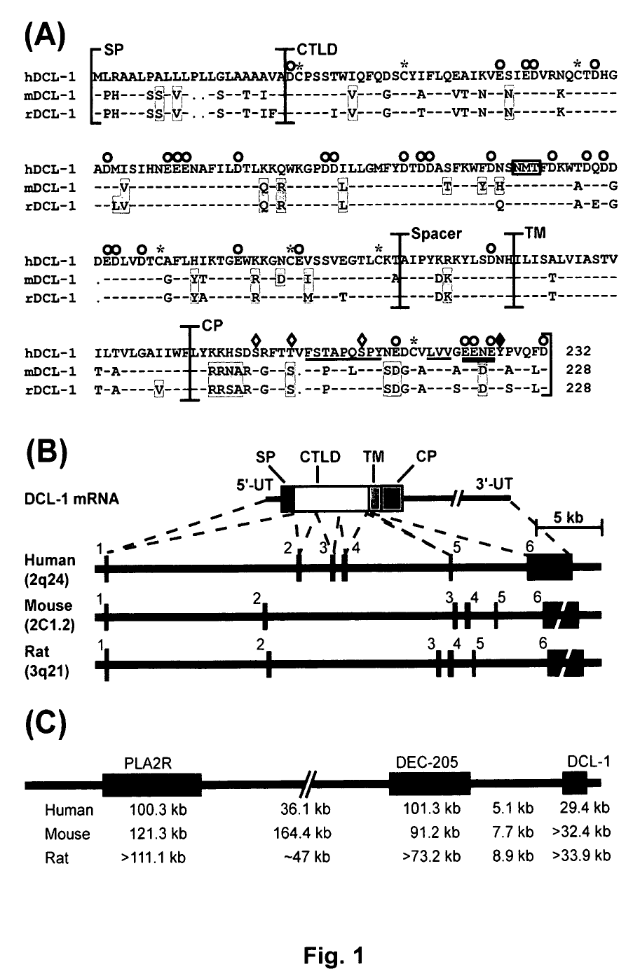

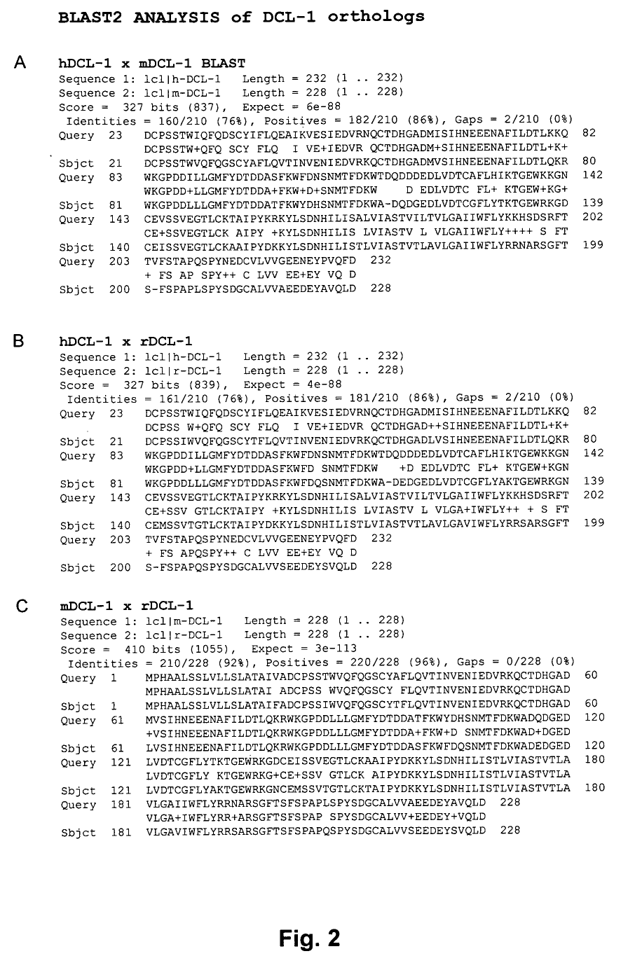

The amino acid comparison between human, mouse (GenBank Accession No. AK004267) and rat (GenBank Accession No. BC089829) DCL-1, indicated that DCL-1 was highly conserved (FIG. 1A and FIG. 2). The overall amino acid identity and similarity between hDCL-1 and the mouse or rat homologue was 76% and 81%, respectively. Mouse DCL-1 was nearly identical to rat DCL-1 (92% identity and 94% similarity).

In addition to six highly conserved cysteines for a typical C-type lectin motif, DCL-1 CTLD were rich in acidic amino acids (D and E), consisting of 21.5%, 19.4% and 18.6% and the predicted pIs were 4.14, 4.24 and 4.21 for human, mouse and rat DCL-1, respectively. One potential N-glycosylation site (NXS/T) was also conserved. In the CP, putative serine/threonine/tyrosine-phosphorylation sites, an acidic amino acid cluster (EEN/DE, potential lysosome targeting signal) (Mahnke, K., M. Guo, S. Lee, H. Sepulveda, S. L. Swain, M. Nussenzweig, and R. M. Steinman. 2000. The dendritic cell receptor for endocytosis, DEC-205, can recycle and enhance antigen presentation via major histocompatibility complex class II positive lysosomal compartments. J Cell Biol. 151:673-684), a hydrophobic amino acid cluster (LVV, potential endocytosis signal) (Sheikh, H., and C. M. Isacke. 1996. A di-hydrophobic Leu-Val motif regulates the basolateral localization of CD44 in polarized Madin-Darby canine kidney epithelial cells. J Biol. Chem. 271:12185-12190) and a putative tyrosine-based internalization motif (FST/PAPQ/LSPY) (East, L., and C. M. Isacke. 2002. The mannose receptor family. Biochim Biophys Acta. 1572:364-386) were all conserved between the species.

To determine the full genomic structure of hDCL-1, we performed a BLAST search of the NCBI genomic sequence database and determined the intron-exon boundaries using the “GT-AG” splice site consensus by comparing the genomic sequence with these cDNA sequences (Breathnach, R., C. Benoist, K. O'Hare, F. Gannon, and P. Chambon. 1978. Ovalbumin gene: evidence for a leader sequence in mRNA and DNA sequences at the exon-intron boundaries. Proc Natl Acad Sci USA. 75.4853-4857, Catterall, J. F., B. W. O'Malley, M. A. Robertson, R. Staden, Y. Tanaka, and G. G. Brownlee. 1978. Nucleotide sequence homology at 12 intron-exon junctions in the chick ovalbumin gene. Nature. 275.510-513). We found the hDCL-1 gene consisted of 6 exons spanning 29 kbp (FIGS. 1B and C). The SP was encoded by exon 1, CTLD was by exon 2-4, the spacer was by exon 5 and the TM and CP were by exon 6. Similarly, both the mouse and rat DCL-1 genes consisted of 6 exons with conserved exon-intron junctions, spanning over 33 kbp. In mice, we detected an alternatively spliced DCL-1 mRNA lacking exon 5 (GenBank Accession No. AK004267), but no such deletion was found in human or rat DCL-1 by extensive BLAST search. The hDCL-1 gene was mapped to the chromosome band 2q24, containing the type I transmembrane C-type lectin cluster, which includes the phospholipase A2 receptor, DEC-205 and DCL-1. This gene cluster was also conserved in mice (2C1.2) and rats (3q21). These data suggested that hDCL-1 protein structure and function was highly conserved during evolution.

### Example 2

### Purification of Leukocytes and Production of MoDC and Mph

Blood was obtained from volunteer donors and “inflamed” palataine tonsils were obtained at routine tonsillectomy with informed consent, as approved by the Mater Hospital Human Research Ethical Committee.

To isolate pure (>98% purity) T, B lymphocytes and NK cells with minimal contaminating cells, cells were first isolated using MACS (AutoMACS, Miltenyi Biotec, North Ryde, NSW, Australia) and then FACS using a FACSVantage (BD Bioscience, Sydney, NSW, Australia) as described previously (Kato, M., K. J. McDonald, S. Khan, I. L. Ross, S. Vuckovic, K. Chen, D. Munster, K. P. MacDonald, and D. N. Hart. 2006. Expression of human DEC-205 (CD205) multilectin receptor on leukocytes. Int Immunol. 18:857-869). T lymphocytes were CD3+CD11c−HLA-DR−. B lymphocytes were CD19+CD20+CD3−CD11c−. NK cells were CD3−CD14−CD19−CD20−CD34−CD11−HLA-DR−CD235a−CD16+CD56+. Monocytes were CD14+CD3−CD20−. Blood dendritic cell (BDC) subsets were CD3−CD14−CD19−CD20−CD34−CD56−CD235a− (Lin−) CD4−CD11c+(myeloid BDC) and Lin−CD4+CD11c− (plasmacytoid BDC).

Monocyte-derived DC (MODC) and macrophages were produced by culturing CD14+ monocytes with GM-CSF and IL-4 (for MODC) or CSF-1 (10,000 U/ml) in RPMI 1640 and 10% AB serum (for Mph) for 5-7 days. As required, the cells were activated with E. coli LPS (Sigma, 100 ng/ml) for 1 day. CSF-1 was a kind gift from David Hume (University of Queensland, QLD, Australia).

### Example 3

### hDCL-1 mRNA Expression Analysis

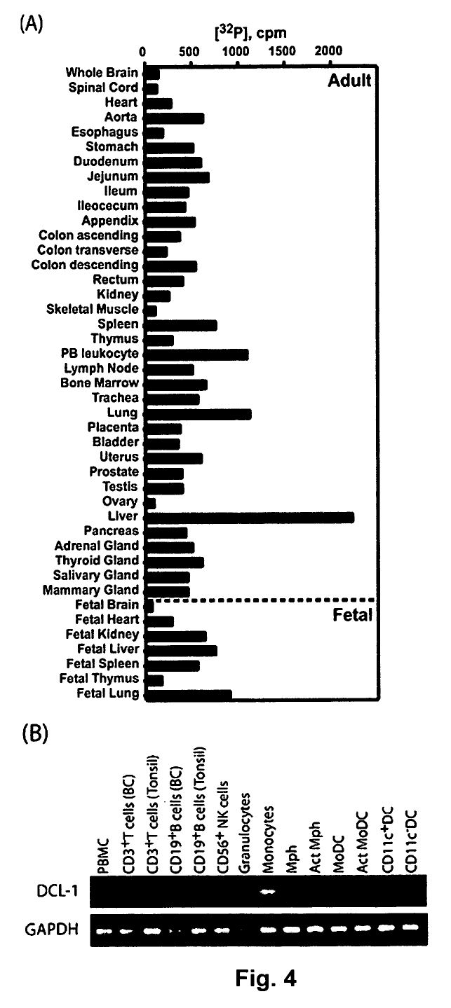

A commercial multiple tissue expression array (MTE Array, Clontech, Palo Alto, Calif.) was probed with [³²P]dCTP-labelled 1.6 kbp hDCL-1 cDNA, produced by PCR using primers MK062 (GACCATGGAGCGGACATGATA: SEQ ID NO: 19) and MK0636 (GGCTCTACCATCTGGGTTTGT: SEQ ID NO: 20) on the pB30-1 plasmid DNA (28) as a template, by random priming (Rediprime II DNA labelling system, Amersham Bioscience, Castle Hill, NSW, Australia). The final washing condition was 0.1×SSC, 5% SDS at 55° C. The blot was quantitated by scintillation counting using a ₃₂P cassette adaptor on a 1450 Microbeta scintillation counter (Wallac, Turku, Finland). Multiple tissue expression array analysis revealed that hDCL-1 mRNA was present in several different issues but at variable levels (FIG. 4A). Adult liver, lung, PBMC and spleen expressed hDCL-1 mRNA at relatively high levels, whereas neuronal tissues (e.g. brain and spinal cord), skeletal muscle and ovary had low levels. In the limited fetal tissues examined, lung, liver, spleen and kidney all had relatively high levels of hDCL-1 mRNA.

Semi-quantitative RT-PCR analysis used cDNA synthesized from RNA obtained from purified leukocytes as described previously (Kato, M., K. J. McDonald, S. Khan, I. L. Ross, S. Vuckovic, K. Chen, D. Munster, K. P. MacDonald, and D. N. Hart. 2006. Expression of human DEC-205 (CD205) multilectin receptor on leukocytes. Int Immunol. 18.857-869). The cDNA was subjected to PCR using hDCL-1 specific primers MK333 (cgggatccGACTACGAAGACCATGACGGT: SEQ ID NO: 21, Bam HI site underlined) and MK203 with p3XFLAG-hDCL-1 as a template, and cloned it into pSecTag B vector (Invitrogen, Melbourne, VIC, Australia) to construct pSec3XFLAG-hDCL-1. Semi-quantitative RT-PCR on dendritic cells (BDC and MoDC), granulocytes, monocytes, macrophages, T lymphocytes, B lymphocytes and NK cells indicated that hDCL-1 mRNA was expressed in MoDC, myeloid and plasmacytoid BDC, monocytes, macrophages and granulocytes but not in T, B lymphocytes or NK cells (FIG. 4B). The mRNA levels in MoDC and macrophages decreased considerably upon activation by LPS.

These data suggested that hDCL-1 expression was restricted to phagocytes, including antigen presenting cells and that its ubiquitous hDCL-1 mRNA expression in human tissues might be explained by the residential tissue phagocytes.

### Example 4

### Construction of DCL-1 Expression Vectors

The pSec3XFLAG-hDCL-1-Ig expression vector was constructed by amplifying the 650 bp fragment encoding the 3×FLAG-hDCL-1 extracellular domain using a T7 primer and MK211 (cgaattcacttacctgtATATTTCCTTTTGTATGGGATAGCT: SEQ ID NO:22, Eco RI site underlined, a splice donor site italicized) and pSec3XFLAG-hDCL-1 as a template and cloned the fragment at the blunted Hind III and Eco RI site of the pcDNA3-Fc vector, which was constructed by cloning a 1.4 kb Hind III-Not I fragment (containing human IgG₁ Fc) from the pIG-1 vector Kato, M., K. J. McDonald, S. Khan, I. L. Ross, S. Vuckovic, K. Chen, D. Munster, K. P. MacDonald, and D. N. Hart. 2006. Expression of human DEC-205 (CD205) multilectin receptor on leukocytes. Int Immunol. 18:857-869 into the pcDNA3 vector (Invitrogen).

### Example 5

### Production hDCL-1 transfectants and hDCL-1-Ig Fusion Protein

CHO-K1 cells were maintained in Ham F12 (Invitrogen, Melbourne, VIC, Australia) and 10% FCS (Invitrogen). The expression vectors were transfected into CHO-K1 cells by electroporation (Genepulser, BioRad, Regent park, NSW, Australia) at 256 V and 950 μF and stable transfectants selected in 400 μg/ml zeocin (Invitrogen). The 3×FLAG-hDCL-1 expressing CHO clone (HB12) was established by single cell sorting with anti-FLAG mAb M2 (Sigma). Similarly, one clone secreting high levels of 3×FLAG-hDCL-1-Ig was chosen by staining with FITC-sheep anti-human IgG, F(ab′)2 (Chemicon, Boronia, VIC, Australia) and cultured in Ham F12 and 3.5% FCS. The 3×FLAG-hDCL-1-Ig was purified from the conditioned medium by protein A column chromatography (Kato, M., K. J. McDonald, S. Khan, I. L. Ross, S. Vuckovic, K. Chen, D. Munster, K. P. MacDonald, and D. N. Hart. 2006. Expression of human DEC-205 (CD205) multilectin receptor on leukocytes. Int Immunol. 18:857-869).

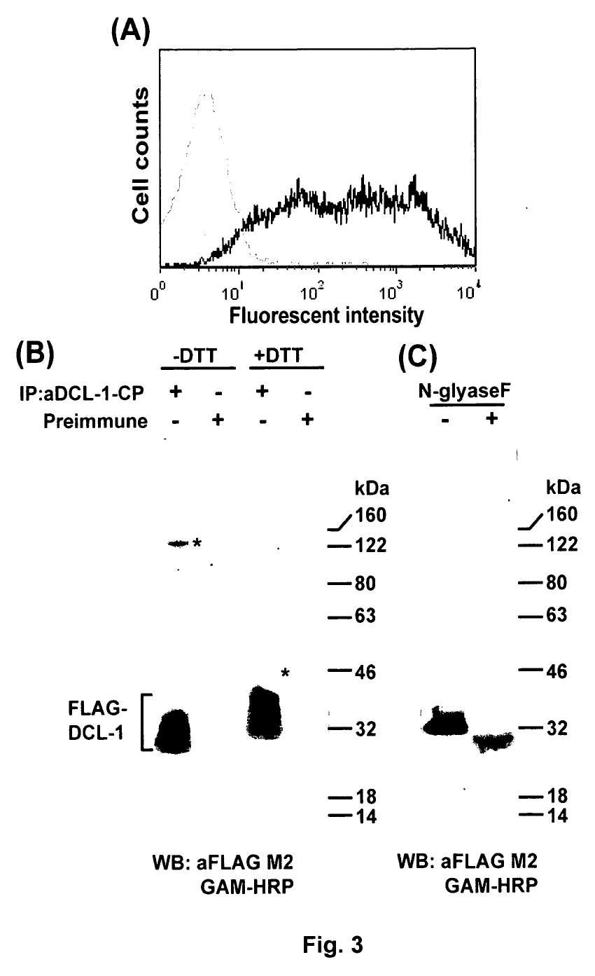

After labelling with the anti-FLAG mAb M2, flow cytometry analysis confirmed significant 3×FLAG-hDCL-1 cell surface expression (FIG. 3A).

### Example 6

### mAb Production to hDCL-1

BALB/C mice were immunized intraperitoneally with 10×10⁶ HB12 cells suspended in PBS and boosted by tail base injections with 3×FLAG-hDCL-1-Ig emulsified with ICFA.

Splenocytes were fused to mouse myeloma cell line NS-1 using a conventional polyethylene glycol fusion protocol. IgG-producing hybridomas selected by dot blot analysis were screened for mAb reactivity to HB12 cells by FACS, their binding to hDCL-1-Ig by ELISA (Kato, M., K. J. McDonald, S. Khan, I. L. Ross, S. Vuckovic, K. Chen, D. Munster, K. P. MacDonald, and D. N. Hart. 2006. Expression of human DEC-205 (CD205) multilectin receptor on leukocytes. Int Immunol. 18:857-869) and immunoprecipitation/Western blot (IP/WB) analysis as described later. The hybridomas (MMRI-18, 19, 20 and 21; all mouse IgG₁ isotype) were adapted to a serum-free medium (Hybridoma-SFM, Invitrogen) and the mAb purified by protein G column chromatography. As required, the mAbs were conjugated with FITC (Sigma) or PE (PhycoLink R-Phycoerythrin Conjugation Kit, Prozyme, San Leandro, Calif.).

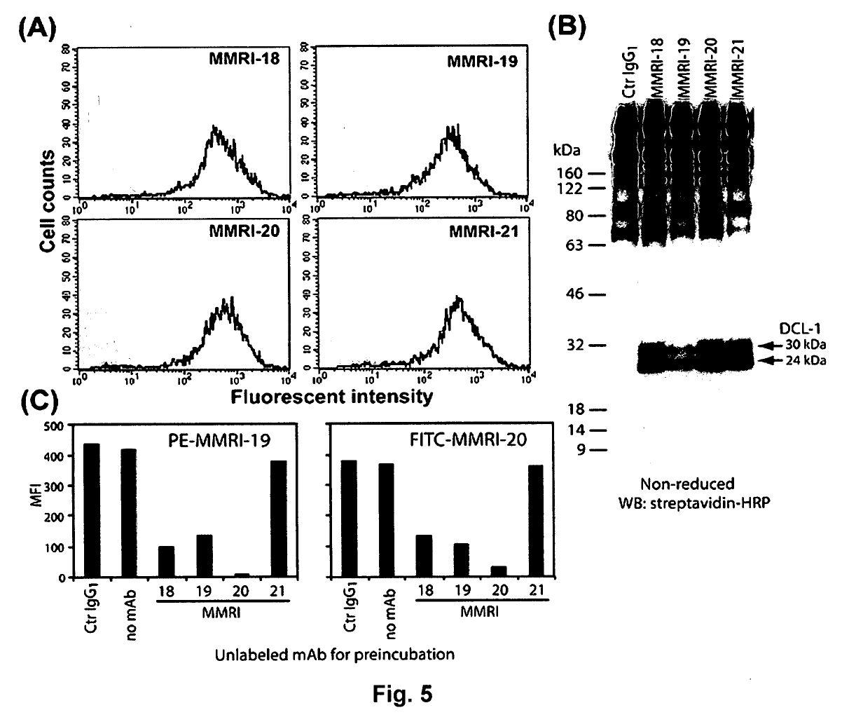

These mAbs labelled HB12 cells (FIG. 5A), bound to 3×FLAG-hDCL-1-Ig fusion protein in ELISA, immunoprecipitated the 3×FLAG-hDCL-1 in IP/WB analysis (data not shown), and also immunoprecipitated 24 and 30 kDa bands (in non-reduced conditions) from cell surface biotinylated PBMC lysate (FIG. 5B).

To map hDCL-1 mAb epitopes, HB12 cells were preincubated with unconjugated hDCL-1 mAbs (10 μg/ml) on ice, washed and stained with 10 μg/ml FITC-conjugated PEMMRI-19 or FITC-MMRI-20 for flow cytometry analysis. Epitope mapping analysis indicated that MMRI-18, 19 and 20 bound to similar epitopes, distinct from that of MMRI-21, as preincubation of HB12 cells with unconjugated MMRI-18, 19 or 20 inhibited the binding of directly conjugated PEMMRI-19 and FITC-20 to the HB12 cells, whereas MMRI-21 preincubation had no effect (FIG. 5C). MMRI-20 preincubation completely blocked PE-MMRI-19 binding, whereas MMRI-18 or 19 preincubation only partially blocked PE-MMRI-20 staining, suggesting that MMRI-20 had the highest affinity among MMRI-18, 19 and 20.

hDCL-1 is Expressed on Phagocytes and DC

Using the new DCL-1 mAbs, we investigated hDCL-1 expression on human leukocytes, including T lymphocytes (CD3+CD11c−HLA-DR−), B lymphocytes (CD20+HLA-DR+CD11c−), NK cells (CD56+HLA-DR−), Mo (CD14+HLA-DR+CD19−) and the myeloid (lin-HLADR+CD11c+) and plasmacytoid (lin-HLA-DR+CD11c− or BDCA2+) BDC subsets using stringent gating strategies (Kato, M., K. J. McDonald, S. Khan, I. L. Ross, S. Vuckovic, K. Chen, D. Munster, K. P. MacDonald, and D. N. Hart. 2006. Expression of human DEC-205 (CD205) multilectin receptor on leukocytes. Int Immunol. 18:857-869). This minimized the possible contamination of myeloid cells (monocyte and myeloid dendritic cells) in cellular aggregates.

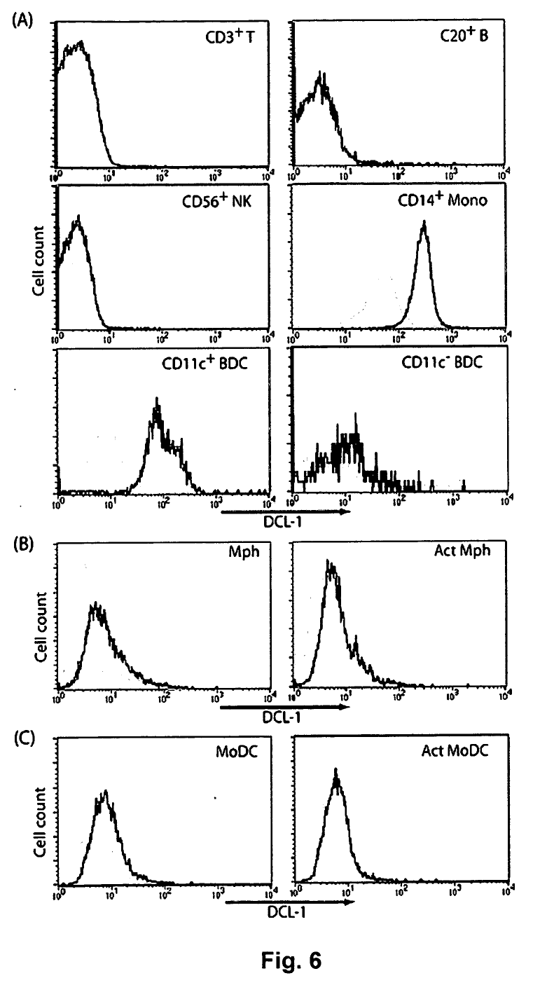

FACS analysis using FITC-MMRI-20 revealed that moderate levels of hDCL-1 were present on both monocyte and myeloid BDC (FIG. 6A). Plasmacytoid BDC expressed low levels of hDCL-1 on their surface.

Granulocytes also expressed hDCL-1 at moderate levels (data not shown). Monocyte derived macrophage and MoDC expressed low levels of hDCL-1 and the levels decreased considerably upon LPS activation (FIGS. 6B and 6C). These data supported the hDCL-1 RNA analysis (see FIG. 3) and showed that hDCL-1 expression was restricted to the phagocytic, monocyte, macrophage, granulocyte and dendritic cell leukocyte populations.

### Example 7

### Immunoprecipitation (IP) and Western Blot (WB) Analysis

Cells were surface-biotinylated using sulfo-NHS-LC-biotin (Pierce, Rockford, Ill.), lysed in a lysis buffer (1% Triton X-100, 0.25% sodium deoxycholate, 0.15 M NaCl, 50 mM Tris-HCl, pH 7.4 and 5 mM EDTA) containing a cocktail of protease inhibitors (Complete, Roche Applied Science, Castle Hill, NSW, Australia and 1 mM PMSF). The lysate was subjected to IP analysis using the DCL-1 mAb and an isotype control mAb 401.21 (Hill, A. S., and J. H. Skerritt. 1989. Monoclonal antibody-based two-site enzyme immunoassays for wheat gluten proteins. 1. Kinetic characteristics and comparison with other ELISA formats. Food Agric. Immunol. 1:147-160) as described previously (Kato, M., K. J. McDonald, S. Khan, I. L. Ross, S. Vuckovic, K. Chen, D. Munster, K. P. MacDonald, and D. N. Hart. 2006. Expression of human DEC-205 (CD205) multilectin receptor on leukocytes. Int Immunol. 18:857-869). For N-deglycosylation, the SDS-PAGE samples were diluted 10 times with 1% CHAPS, 1 mM PMSF and digested with 10 U of N-glycosidase F (Roche Applied Science) at 37° C. overnight. The samples were concentrated with Microcon YM30 ultrafiltration units (Millipore) and subjected to SDS-PAGE.

For IP/WB analysis, five to ten million cells were lysed with 1 ml of the lysis buffer. Two different concentrations of the cell lysate (final protein concentration: 400 and 133 μg/ml) were immunoprecipitated with the rabbit peptide antibody to hDCL-1 cytoplasmic domain and protein A Sepharose as described previously (Kato, M., S. Khan, N. Gonzalez, B. P. O'Neill, K. J. McDonald, B. J. Cooper, N. Z. Angel, and D. N. Hart. 2003. Hodgkin's lymphoma cell lines express a fusion protein encoded by intergenically spliced mRNA for the multilectin receptor DEC-205 (CD205) and a novel C-type lectin receptor DCL-1. J Biol. Chem. 278:34035-34041), Western-blotted with MMRI-20, followed by chemiluminescence detection.

Immunoprecipitation with rabbit anti-hDCL-1 CP peptide antibody (Kato, M., S. Khan, N. Gonzalez, B. P. O'Neill, K. J. McDonald, B. J. Cooper, N. Z. Angel, and D. N. Hart. 2003. Hodgkin's lymphoma cell lines express a fusion protein encoded by intergenically spliced mRNA for the multilectin receptor DEC-205 (CD205) and a novel C-type lectin receptor DCL-1. J Biol. Chem. 278.34035-34041) and Western blotting with anti-FLAG mAb, identified 3×FLAG-hDCL-1 as a broad band with a modal size 32 kDa in non-reduced and 35 kDa in reduced conditions (FIG. 3B), confirming the presence of the intermolecular disulfide bonds expected in a C-type lectin domain (Weis, W. I., M. E. Taylor, and K. Drickamer. 1998. The C-type lectin superfamily in the immune system. Immunol Rev. 163:19-34). N-glycosidase F treatment focused the 3XFLAGhDCL-1 into a more defined single band of 30 kDa when reduced (FIG. 3C), indicating that the DCL-1 CTLD N-glycosylation motif was glycosylated in CHO cells. The 3×FLAG-hDCL-1 was also glycosylated in transfected COS-1 and HEK293 cells (data not shown).

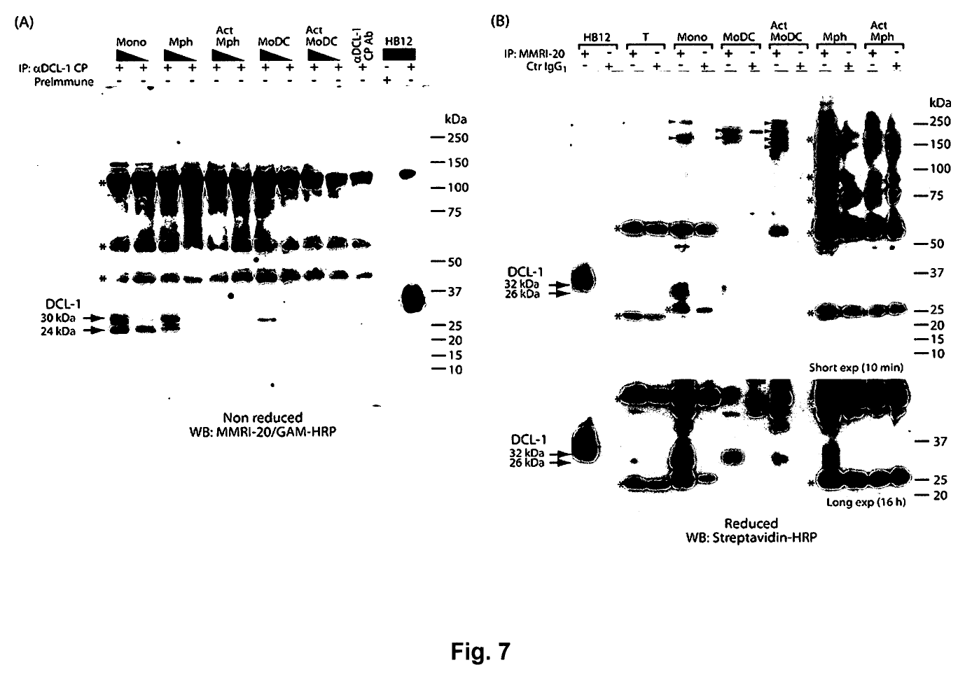

We used semiquantitative IP/WB to analyze DCL-1 expression on monocyte, macrophage and MoDC (FIG. 7A)(Kato, M., K. J. McDonald, S. Khan, I. L. Ross, S. Vuckovic, K. Chen, D. Munster, K. P. MacDonald, and D. N. Hart. 2006. Expression of human DEC-205 (CD205) multilectin receptor on leukocytes. Int Immunol. 18:857-869). We identified two bands in monocyte, macrophage and MoDC of 24 and 30 kDa in non-reducing conditions. The ratios of these two bands differed, depending on the cell type: the 24 kDa band was more abundant than the 30 kDa band in monocyte, whereas the 30 kDa band was more prominent in macrophage and MoDC than the 24 kDa band. LPS activation consistently decreased both signal levels. The exact nature of these two hDCL-1 bands requires future clarification.

To find potential hDCL-1 associated proteins, we immunoprecipitated lysate from cell surface biotinylated leukocytes with MMRI-20 and used a streptavidin probe to reveal the proteins (FIG. 7B). In addition to the expected hDCL-1 bands (26 and 32 kDa in reducing conditions), MMRI-20 coimmunoprecipitated additional bands (50, 150-200 kDa) from the monocyte and MoDC lysate, suggesting that hDCL-1 was associated with these molecules at their cell surfaces. We could not reduce the background signal levels from macrophage sufficiently to unequivocally define similar discrete protein bands, despite several attempts, but protein bands with similar molecular mass were also detected in anti hDCL-1 immunoprecipitation from macrophage.

### Example 8

### DCL-1 Expression Analysis by Flow Cytometry

HB12 cells were stained with anti-FLAG mAb M2 or anti-DCL-1 mAb followed by FITCsheep anti mouse IgG, F(ab′)2 (Chemicon) in cold PBS with 2 mM EDTA, 0.5% (w/v) BSA (MACS buffer), and subjected to FACS using a FACSCalibur (BD Bioscience). PBMC, MoDCs and macrophages were suspended in cold MACS buffer and stained with FITC-MMRI-20 or an isotype control mAb 401.21 (10 μg/ml) in combination with fluorochrome-conjugated lineage antibodies (Kato, M., K. J. McDonald, S. Khan, I. L. Ross, S. Vuckovic, K. Chen, D. Munster, K. P. MacDonald, and D. N. Hart. 2006. Expression of human DEC-205 (CD205) multilectin receptor on leukocytes. Int Immunol. 18.857-869). T lymphocytes were defined as CD3+ CD11c−HLA-DR−; B lymphocytes were CD20+HLA-DR+CD11c−; NK cells were CD56+HLA-DR−; monocytes were CD14+HLA-DR+CD19−; myeloid BDC were HLA-DR+Lin−CD11c+; plasmacytoid BDC were HLA-DR+Lin−CD11c− or BDCA2+CD11c−.

### Example 9

### DCL-1 Endocytosis by HB12 Cells

Near confluent HB12 cells cultured for 36-48 h in a 24 well plate were incubated at 37° C. in a CO₂ incubator for indicated time periods with FITC-MMRI-20 or 401.21 (10 μg/ml) diluted in Ham F12, 10% FCS and 10 mM HEPES, pH 7.4. For t=0 min, the cells were stained on ice for 1 h with the FITC-conjugated mAbs. At the end of incubation, the cells were chilled on ice, washed once with cold culture medium and harvested in cold MACS buffer. The cells were stained with biotinylated MMRI-21 (2.5 μg/ml) on ice, followed by allophcocyaninstreptavidin (BD Bioscience) to detect cell surface hDCL-1, fixed with 4% paraformaldehyde (PFA) in PBS and analyzed by FACS.

Geometrical mean of fluorescence (MFI) was determined using FCS Express version 3 software (De Novo Software, Thornhill, Ontario, Canada), and relative hDCL-1 expression was calculated using the hDCL-1 cell surface expression at t=0 min as 100%.

For confocal microscope analysis, HB12 cells cultured on round cover slips (13 mm in diameter) were incubated with FITC-MMRI-20 or 401.21 as above. At the end of incubation, the cells were chilled on ice, washed twice with cold culture medium and stained with biotinylated MMRI-21 followed by streptavidin-Alexafluor 633 (AF₆₃₃, Invitrogen) in the cold medium. After fixing with PFA, the cells were permeabilized with 0.1% Triton X-100 in HEPES-buffered saline (HBS: 1 mM CaCl₂, 1 mM MgCl₂, 0.15 M NaCl, 10 mM HEPES, pH 7.4), stained with AF₅₄₆-phalloidin (Invitrogen) and DAPI, postfixed with 4% PFA in HBS, mounted with Prolong Gold (Invitrogen) and observed under a laser-scanning confocal microscope (LSM) using a 100× objective (LSM510 META, Carl Zeiss, North Ryde, NSW, Australia).

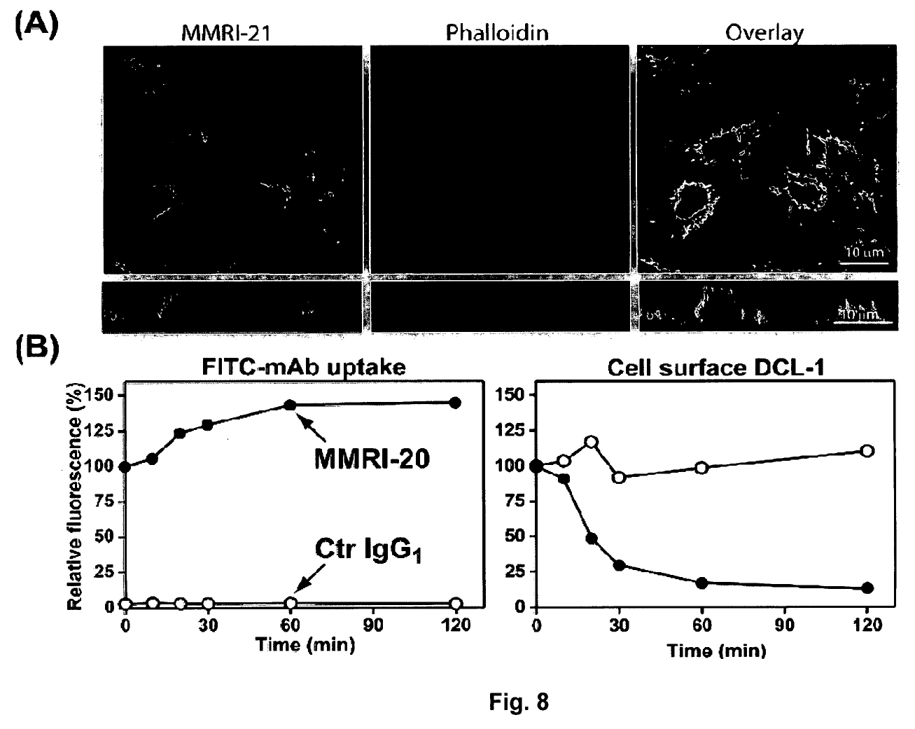

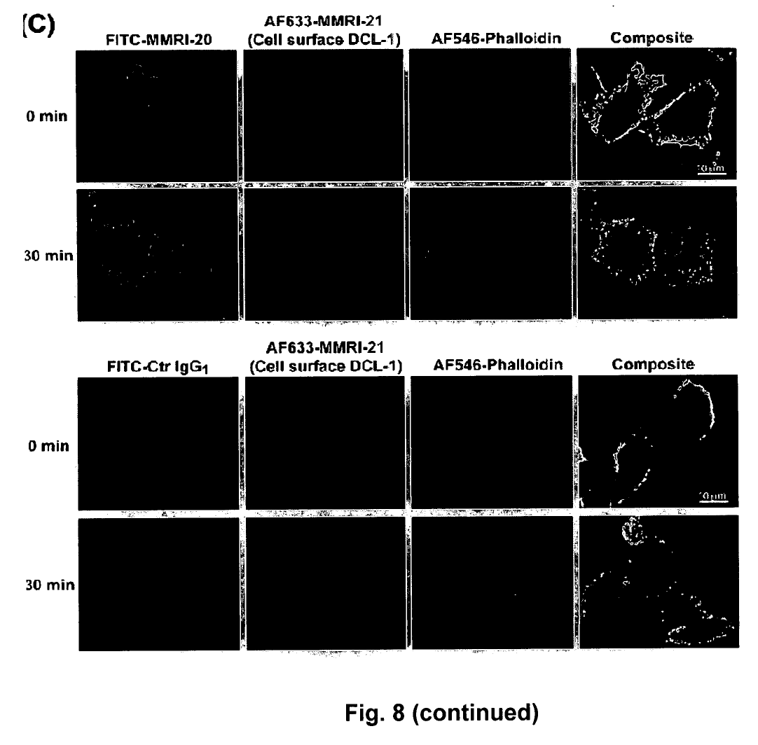

Cellular localization of hDCL-1 in HB12 cells and its relationship with F-actin was assessed by LSM (FIG. 8A). Both x-y and x-z optical sectioning of the staining indicated that the majority of hDCL-1 protein in HB12 cells co-localized with F-actin in filopodia and cellular cortex at basal surfaces (x-y sectioning) and cellular cortex (x-z sectioning), indicating that hDCL-1 was associated with F-actin.

Because hDCL-1 CP contained potential internalization motifs (tyrosine-based and hydrophobic amino acid-based), we investigated hDCL-1 internalization by HB12 cells (FIGS. 8B and 8C) using flow cytometry and laser scanning confocal microscopy. When incubated with FITC-MMRI-20 at 37° C., the cells took up the antibody for up to 60 min, then reached a plateau. The increase of FITC-MMRI-20 uptake was due to the continuous transport of intracellular hDCL-1 to cell surface. This uptake was specific because the FITC-isotype control mAb was not taken up by the cells. In contrast, the cell surface hDCL-1 detected with biotinylated MMRI-21, which bound to a distinct epitope from that of MMRI-20 (FIG. 5C), decreased concomitantly, indicating that hDCL-1 was endocytosed when bound to MMRI-20. The half life of cell surface hDCL-1 was ˜20 min. The level of cell surface hDCL-1 was relatively unchanged, when HB12 cells were incubated with the control mAb. These flow cytometric data were confirmed by laser scanning confocal microscopy, showing that hDCL-1 endocytosis was hDCL-1 mAb specific. Interestingly, endocytosed hDCL-1 at t=30 min no longer co-localized with F-actin, whilst cell surface hDCL-1 co-localized with F-actin at t=0 min (FIG. 5C).

These data indicated that (i) cell surface hDCL-1 is endocytosed when bound by hDCL-1 mAb, (ii) intracellular hDCL-1 (up to 50% of cell surface hDCL-1) was transported to the cell surface and internalized upon hDCL-1 mAb binding and (iii) once internalized, hDCL-1 did not co-localize with F-actin and was not recycled to cell surface for at least up to 120 min.

### Example 10

### Phagocytosis of MMRI-20-Coated Microbeads by HB12 Cells

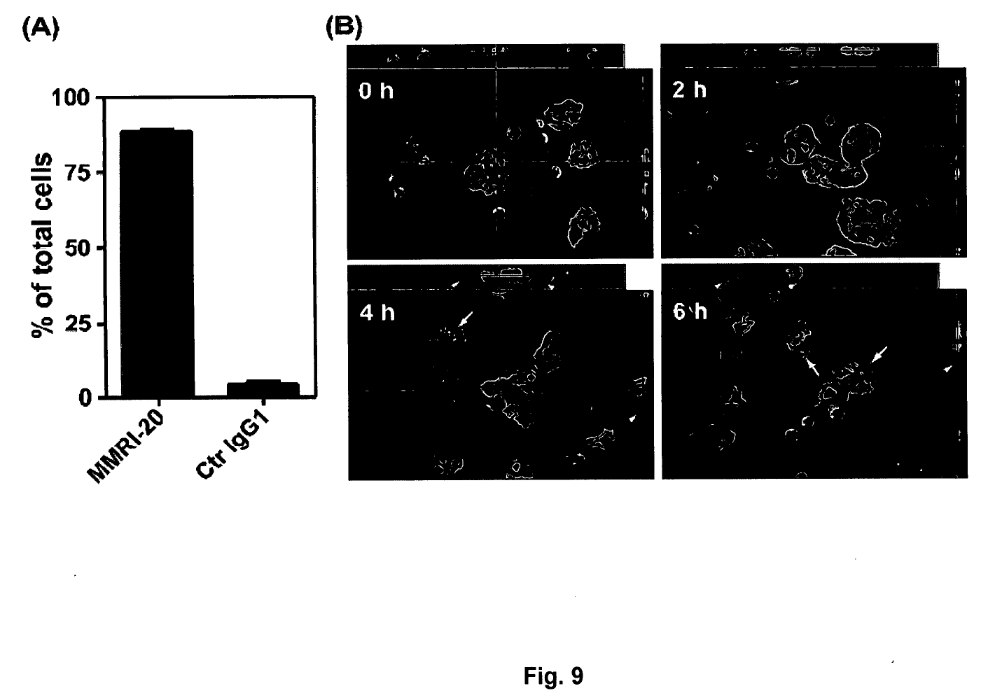

One of the presumed functions of C-type lectins on phagocytes and dendritic cells is phagocytosis. Therefore, we investigated whether hDCL-1 behaved as a phagocytic receptor using HB12 cells (FIG. 9A).

Rat anti-mouse IgG₁-microbeads (4.5 μm in diameter, Dynabeads, Invitrogen) were incubated with a saturating concentration of MMRI-20 or the isotype control mAb (10 μg mAb/100 μl beads suspension), washed and resuspended in the CHO cell culture medium. HB12 cells cultured on round cover slips were incubated on ice with the microbeads for 1 h to allow the cells to bind the beads. After washing extensively with the cold culture medium, the cells were incubated at 37° C. for indicated periods, washed with cold HBS, fixed with PFA and permeabilized as above.

The cells were stained with AF₄₈₈-goat anti-mouse IgG, F(ab)₂ (GAM, Invitrogen), AF₅₄₆-phalloidin and DAPI, postfixed and subjected to LSM.

To quantitate the microbeads bound on HB12 cells at t=0 h, the cells incubated with the microbeads on ice (in triplicates) were harvested with cold MACS buffer, stained with AF₄₈₈-GAM and subjected to FACS analysis.

As expected, the majority of HB12 cells (>85%) bound the MMRI-20-coated microbeads, whereas little binding was observed with the isotype control mAb-coated beads. When the cells were incubated at 37° C., we found that HB12 cells phagocytosed the MMRI-20-coated beads (FIG. 9B): At t=0 h, we observed colocalization of F-actin at the contacts between beads and cells. In 2-4 h, HB12 cells began to phagocytose the majority of the microbeads surrounded by phagocytic cups. In some cases, the microbeads were fully phagocytosed. Occasionally, we observed AF488 dye released in the cytoplasm, suggesting that some beads were transported to phagolysosomes for proteolytic degradation. These data indicated that hDCL-1 behaves as a phagocytic receptor.

### Example 11

### Binding of Anti-C-Type Lectin mAb-Coated Microbeads to Mph

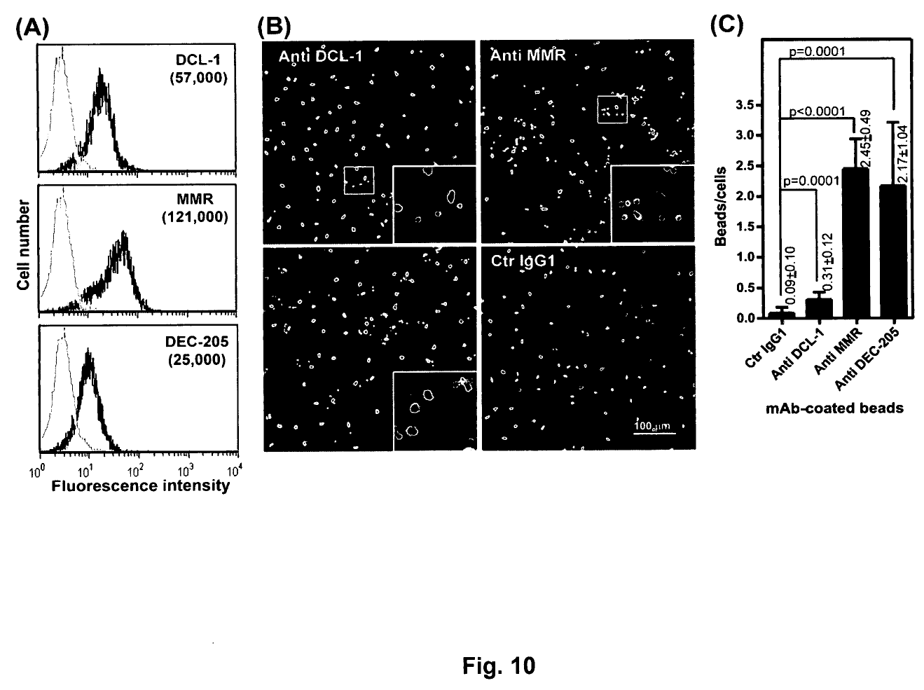

Mph are prototypical phagocytes and express an array of C-type lectin receptors, including MMR and DEC-205, which may play a role in phagocytosis. The surface expression of these C-type lectin receptors on macrophages was assessed using a quantitative indirect immunofluorescence analysis (Serke, S., A. van Lessen, and D. Huhn. 1998. Quantitative fluorescence flow cytometry: a comparison of the three techniques for direct and indirect immunofluorescence. Cytometry. 33:179-187; Poncelet, P., and P. Carayon. 1985. Cytofluorometric quantification of cell-surface antigens by indirect immunofluorescence using monoclonal antibodies. J Immunol Methods. 85:65-74) (FIG. 10A). The analysis revealed that, although the expression levels varied among donors, MMR was the most abundant C-type lectin receptor, whereas the levels of DCL-1 and DEC-205 were approximately ½ and ⅕ that of the MMR, respectively. Rat anti-mouse IgG₁-conjugated microbeads were coated with anti-DEC-205 mAb (MMRI-7) (Kato, M., K. J. McDonald, S. Khan, I. L. Ross, S. Vuckovic, K. Chen, D. Munster, K. P. MacDonald, and D. N. Hart. 2006. Expression of human DEC-205 (CD205) multilectin receptor on leukocytes. Int Immunol. 18.857-869), anti-MMR mAb (clone 15-2, Abcam, Cambridge, Mass.) (33) or DCL-1 (MMRI-20) as above, washed and resuspended in RPMI 1640, 1% BSA and 10 mM HEPES, pH7.4 (Macrophage binding buffer). Macrophages cultured on cover slips were incubated on ice with the beads in the binding buffer for 1 h. The cells were washed extensively with the binding buffer, fixed and permeabilized as above.

The cells were stained with AF₄₈₈-GAM, AF₅₄₆-phalloidin and DAPI, and subjected to LSM using a 20× objective. Randomly selected field images (10-20 fields/sample) were taken, and the numbers of beads associated to the cells were counted. Little or no staining (0.09+0.10 beads/cells, n=20) of an isotype control mAb-coated beads was seen. Statistical significance was assessed by Student's t-test (2-tailed, unpaired) using GraphPad Prism software (GraphPad Software, San Diego, Calif.).

The results of this analysis, which are presented in FIGS. 10B and C, show that anti-MMR and anti-hDEC-205 mAb-coated microbeads bound to Mph effectively and more than two microbeads were found to be associated with Mph (2.45+0.49 and 2.17+1.04 beads/cells, respectively, mean±SD, n=10). In contrast, only a small number of MMRI-20-coated microbeads bound to Mph (0.31+0.12 beads/cell, n=10). The binding of C-type lectin mAb-coated beads to Mph compared to isotype control mAb microbeads was statistically significant (p<0.0001 by Student's t test).

The comparative p-values for MMRI-20 versus anti-MMR mAb-coated beads and MMRI-20 versus anti-hDEC-205 mAb coated beads were most significant at 9×10⁻⁸ and 3×10⁻⁴, respectively, but there was no significant difference between the binding of anti-MMR mAb and anti-DEC-205 mAb-coated beads to Mph (p=0.46).

After incubation at 37° C. for 3 h, the C-type lectin mAb-coated beads were phagocytosed completely (data not shown). These data indicated that macrophages could utilize hDCL-1 for particle binding and phagocytosis, although DCL-1 was less efficient than MMR or hDEC-205-mediated binding and uptake.

### Example 12

### Cellular Localization of C-Type Lectins in Mph

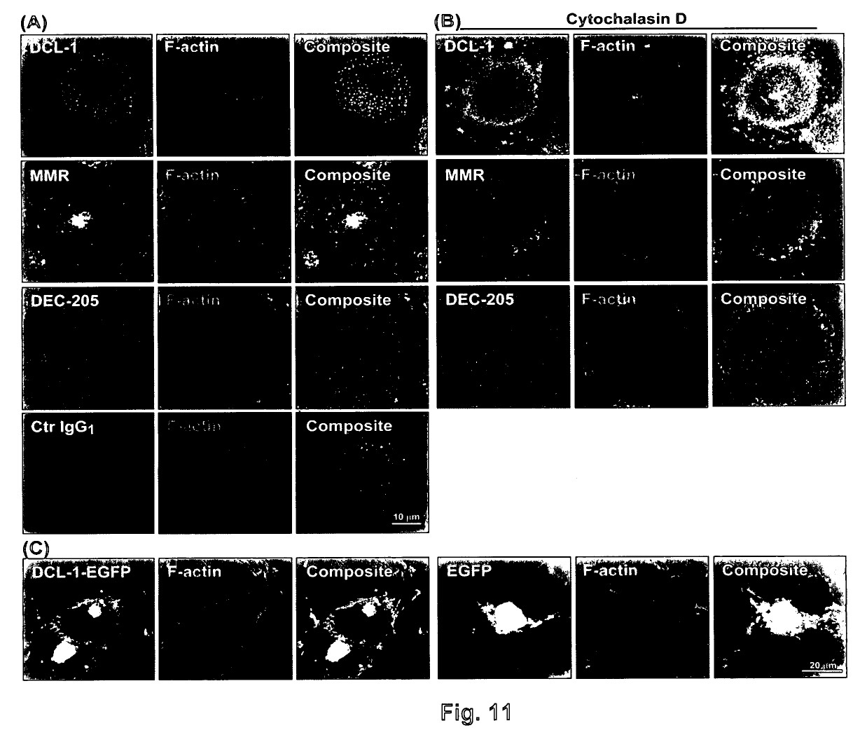

It was possible that the relatively inefficient binding and subsequent phagocytosis of hDCL-1 mAb-coated microbeads was due to a distinct and/or alternative hDCL-1 cellular localization from that of MMR or DEC-205. The inventor therefore investigated the localization of hDCL-1 and MMR in relation to F-actin in fixed and permeabilized Mph using LSM (FIG. 11). Staining with MMRI-20 and AF₅₄₆-phalloidin showed that hDCL-1 colocalized with F-actin structures at the near basal surface such as filopodia and lamellipodia (not shown) in periphery (FIG. 11A). The inventors also found hDCL-1 staining in relatively large dot-like structures (1-2 μm) or podosomes, associated with F-actin in some Mph. In contrast, MMR and DEC-205 were dispersed and there was no apparent colocalization of MMR or DEC-205 with F-actin (FIG. 11A). The hDCL-1 colocalization with F-actin became more apparent when Mph were treated with cytochalasin D to disrupt F-actin extension (FIG. 11B). The treatment resulted in formation of F-actin clumps at the periphery of the Mph and the marked proportion of hDCL-1 colocalized with the clumps, whilst there was no colocalization of MMR or DEC-205 with clumped F-actin.

To further investigate whether the colocalization of hDCL-1 to F-actin is intrinsic to hDCL-1 protein, the inventors constructed an expression vector for hDCL-1-EGFP fusion protein (pEGFP-hDCL-1) and transiently transfected it into COS-1 cells (FIG. 11C). The hDCL-1-EGFP colocalized with F-actin at the cellular cortex and microvilli of the apical cell surface of the transfectants, whereas control EGFP expression was restricted within the cytoplasm and nuclei. These data indicate that the DCL-1 colocalizes intrinsically with F-actin involved in cell adhesion and migration, suggesting that hDCL-1 may play an additional or alternative role in Mph adhesion and migration.

### Example 13

DNA Sequencing and Bioinformatics

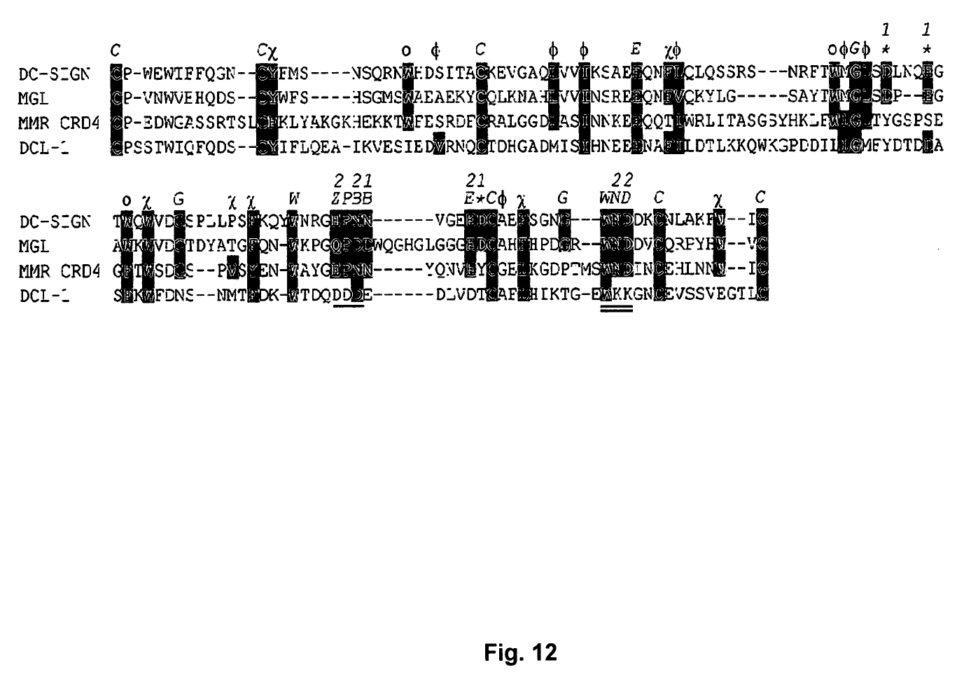

The DNA sequences of the expression vectors were confirmed by sequencing (Australian Genome Research Facility, St Lucia, QLD, Australia). DCL-1 homologues were identified using non-redundant database (nr) and EST database from the National Center for Biotechnology Information (NCBI) by performing a BLASTn search using human DCL-1 coding sequence (GenBank Accession No. AY314007) for inquiry. Multiple sequence alignment was performed using ClustalW available on the Australian National Genomic Information Service Bioinformatics service (ANGIS, at the Word Wide Web at angis.org.au) see FIG. 12. Potential Serine/threonine/tyrosine phosphorylation sites and pI were predicted using the programs NetPhos 2.0 (Blom, N., S. Gammeltoft, and S. Brunak. 1999. Sequence and structure-based prediction of eukaryotic protein phosphorylation sites. J Mol. Biol. 294:1351-1362) and ProtParam, respectively, on the ExPASy Molecular Biology server (http://au.expasy.org) (Gasteiger, E., A. Gattiker, C. Hoogland, I. Ivanyi, R. D. Appel, and A. Bairoch. 2003. ExPASy: The proteomics server for in-depth protein knowledge and analysis. Nucleic Acids Res. 31:3784-3788).

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

What is claimed is:

1. A method for modulating an immune function of a cell that expresses DCL-1, the method comprising exposing the cell to an agent that modulates the level or functional activity of DCL-1, wherein the agent is selected from the group consisting of:
a) a proteinaceous molecule comprising an amino acid sequence which has at least 75% sequence identity to the sequence set forth in any one of SEQ ID NOs: 8, 12, 15 or 16 and which modulates at least one immune function selected from the group consisting of endocytosis, phagocytosis, cell adhesion and cell migration;
b) a proteinaceous molecule comprising an amino acid sequence which is encoded by a nucleotide sequence that hybridizes under high stringency conditions to the sequence set forth in any one of SEQ ID NOs: 7, 10, 11, 13, 14, 17 or 18 and which modulates at least one immune function selected from the group consisting of endocytosis, phagocytosis, cell adhesion and cell migration;
c) an antibody or fragment thereof which specifically binds to the amino acid sequence defined in a) or b);
d) a nucleic acid molecule comprising a nucleotide sequence that encodes the amino acid sequence defined in a) or b);
e) a nucleic acid molecule comprising a nucleic acid sequence that hybridizes under high stringency conditions to the nucleotide sequence defined in d); and
f) a nucleic acid molecule which comprises a nucleotide sequence that is antisense to the nucleotide sequence defined in d) or e).

2. The method of claim 1, wherein the immune function is endocytosis.

3. The method of claim 1, wherein the immune function is phagocytosis.

4. The method of claim 1, wherein the immune function is cell adhesion.

5. The method of claim 1, wherein the immune function is cell migration.

6. A method of modulating an immune response, comprising exposing a cell that expresses DCL-1 to an agent that modulates the level or functional activity of DCL-1, wherein the agent is selected from the group consisting of:
a) a proteinaceous molecule comprising an amino acid sequence which has at least 75% sequence identity to the sequence set forth in any one of SEQ ID NOs: 8, 12, 15 or 16 and which modulates at least one immune function selected from the group consisting of endocytosis, phagocytosis, cell adhesion and cell migration;
b) a proteinaceous molecule comprising an amino acid sequence which is encoded by a nucleotide sequence that hybridizes under high stringency conditions to the sequence set forth in any one of SEQ ID NOs: 7, 10, 11, 13, 14, 17 or 18 and which modulates at least one immune function selected from the group consisting of endocytosis, phagocytosis, cell adhesion and cell migration;
c) an antibody or fragment thereof which specifically binds to the amino acid sequence defined in a) or b);
d) a nucleic acid molecule comprising a nucleotide sequence that encodes the amino acid sequence defined in a) or b);
e) a nucleic acid molecule comprising a nucleotide sequence that hybridizes under high stringency conditions to the nucleotide sequence defined in d);
f) a nucleic acid molecule which comprises a nucleotide sequence that is antisense to the nucleotide sequence defined in d) or e); and
g) an inhibitory RNA molecule that is specific to the nucleotide sequence defined in d) or e).

7. A method of treating or preventing a disease associated with an aberrant immune response in a subject, the method comprising administering to the subject an immune response-modulating effective amount of an agent that modulates the level or functional activity of DCL-1, wherein the agent is selected from the group consisting of:
a) a proteinaceous molecule comprising an amino acid sequence which has at least 75% sequence identity to the sequence set forth in any one of SEQ ID NOs: 8, 12, 15 or 16 and which modulates at least one immune function selected from the group consisting of endocytosis, phagocytosis, cell adhesion and cell migration;
b) a proteinaceous molecule comprising an amino acid sequence which is encoded by a nucleotide sequence that hybridizes under high stringency conditions to the sequence set forth in any one of SEQ ID NOs: 7, 10, 11, 13, 14, 17 or 18 and which modulates at least one immune function selected from the group consisting of endocytosis, phagocytosis, cell adhesion and cell migration;
c) an antibody or fragment thereof which specifically binds to the amino acid sequence defined in a) or b);
d) a nucleic acid molecule comprising a nucleotide sequence that encodes the amino acid sequence defined in a) or b);
e) a nucleic acid molecule comprising a nucleotide sequence that hybridizes under high stringency conditions to the nucleotide sequence defined in d);
f) a nucleic acid molecule which comprises a nucleotide sequence that is antisense to the nucleotide sequence defined in d) or e); and
g) an inhibitory RNA molecule that is specific to the nucleotide sequence defined in d) or e).

8. A method of treating or preventing a disease associated with an unwanted immune response in a subject, the method comprising administering to the subject an immune response-modulating effective amount of an agent that modulates the level or functional activity of DCL-1, wherein the agent is selected from the group consisting of:
a) a proteinaceous molecule comprising an amino acid sequence which has at least 755% sequence identity to the sequence set forth in any one of SEQ ID NOs: 8, 12, 15 or 16 and which modulates at least one immune function selected from the group consisting of endocytosis, phagocytosis, cell adhesion and cell migration;
b) a proteinaceous molecule comprising an amino acid sequence which is encoded by a nucleotide sequence that hybridizes under high stringency conditions to the sequence set forth in any one of SEQ ID NOs: 7, 10, 11, 13, 14, 17 or 18 and which modulates at least one immune function selected from the group consisting of endocytosis, phagocytosis, cell adhesion and cell migration;
c) an antibody or fragment thereof which specifically binds to the amino acid sequence defined in a) or b);
d) a nucleic acid molecule comprising a nucleotide sequence that encodes the amino acid sequence defined in a) or b);
e) a nucleic acid molecule comprising a nucleotide sequence that hybridizes under high stringency conditions to the nucleotide sequence defined in d);
f) a nucleic acid molecule which comprises a nucleotide sequence that is antisense to the nucleotide sequence defined in d) or e); and
g) an inhibitory RNA molecule that is specific to the nucleotide sequence defined in d) or e).

9. The method according to claim 7 or claim 8, wherein the disease is cancer.

10. The method according to claim 7 or claim 8, wherein the disease is an infectious disease.

11. The method according to claim 7 or claim 8, wherein the disease is associated with an unwanted or deleterious immune response.

12. The method according to claim 7 or claim 8, wherein the agent is the antibody or antibody fragment, which is coupled to, or otherwise associated with, an antigen that corresponds to at least a portion of a target antigen that associates with the disease.

13. The method of any one of claims 1, 6, 7 or 8, wherein the proteinaceous molecule of a) comprises an amino acid sequence which has at least 80% sequence identity to the sequence set forth in any one of SEQ ID NOs: 8, 12, 15 or 16.

14. The method of any one of claims 1, 6, 7 or 8, wherein the proteinaceous molecule of a) comprises an amino acid sequence which has at least 85% sequence identity to the sequence set forth in any one of SEQ ID NOs: 8, 12, 15 or 16.

15. The method of any one of claims 1, 6, 7 or 8, wherein the proteinaceous molecule of a) comprises an amino acid sequence which has at least 90% sequence identity to the sequence set forth in any one of SEQ ID NOs: 8, 12, 15 or 16.

16. The method of any one of claims 1, 6, 7 or 8, wherein the proteinaceous molecule of a) comprises an amino acid sequence which has at least 95% sequence identity to the sequence set forth in any one of SEQ ID NOs: 8, 12, 15 or 16.

17. A method of screening an agent for ability to modulate an immune response, comprising:
a) contacting a cell expressing a nucleic acid molecule that comprises (a) a nucleotide sequence encoding an amino acid sequence which has at least 75% sequence identity to the sequence set forth in any one of SEQ ID NOs: 8, 12, 15 or 16 and which modulates at least one immune function selected from the group consisting of endocytosis, phagocytosis, cell adhesion and cell migration; or (b) a nucleotide sequence that hybridizes under high stringency conditions to the sequence set forth in any one of SEQ ID NOs: 7, 10, 11, 13, 14, 17 or 18 and which encodes an amino acid sequence that modulates at least one immune function selected from the group consisting of endocytosis, phagocytosis, cell adhesion and cell migration, with the agent; and
b) detecting a change in the level and/or activity of an expression product of the nucleic acid molecule, relative to a normal or reference level and functional activity in the absence of the agent, wherein the change indicates that the agent modulates the immune response.

18. The method of claim 17, wherein the nucleic acid molecule expressed by the cell of a) encodes an amino acid sequence which has at least 75% sequence identity to the sequence set forth in any one of SEQ ID NOs: 8, 12, 15 or 16.

19. The method of claim 17, wherein the nucleic acid molecule expressed by the cell of a) encodes an amino acid sequence which has at least 80% sequence identity to the sequence set forth in any one of SEQ ID NOs: 8, 12, 15 or 16.

20. The method of claim 17, wherein the nucleic acid molecule expressed by the cell of a) encodes an amino acid sequence which has at least 85% sequence identity to the sequence set forth in any one of SEQ ID NOs: 8, 12, 15 or 16.

21. The method of claim 17, wherein the nucleic acid molecule expressed by the cell of a) encodes an amino acid sequence which has at least 90% sequence identity to the sequence set forth in any one of SEQ ID NOs: 8, 12, 15 or 16.

22. The method of claim 17, wherein the nucleic acid molecule expressed by the cell of a) encodes an amino acid sequence which has at least 95% sequence identity to the sequence set forth in any one of SEQ ID NOs: 8, 12, 15 or 16.


SEQUENCE LISTING (31 sequences)

SEQ ID NO: 1 - DNA, 5622 bp, Homo sapiens
atgaggacaggctgggcgacccctcgccgcccggcggggctcctcatgctgctcttctggttcttcgatctcgcggagccctctggccgcgcagctaatgaccccttcaccatcgtccatggaaatacgggcaagtgcatcaagccagtgtatggctggatagtagcagacgactgtgatgaaactgaggacaagttatggaagtgggtgtcccagcatcggctctttcatttgcactcccaaaagtgccttggcctcgatattaccaaatcggtaaatgagctgagaatgttcagctgtgactccagtgccatgctgtggtggaaatgtgagcaccactctctgtacggagctgcccggtaccggctggctctgaaggatggacatggcacagcaatctcaaatgcatctgatgtctggaagaaaggaggctcagaggaaagcctttgtgaccagccttatcatgagatctataccagagatgggaactcttatgggagaccttgtgaatttccattcttaattgatgggacctggcatcatgattgcattcttgatgaagatcatagtgggccatggtgtgccaccaccttaaattatgaatatgaccgaaagtggggcatctgcttaaagcctgaaaacggttgtgaagataattgggaaaagaacgagcagtttggaagttgctaccaatttaatactcagacggctctttcttggaaagaagcttatgtttcatgtcagaatcaaggagctgatttactgagcatcaacagtgctgctgaattaacttaccttaaagaaaaagaaggcattgctaagattttctggattggtttaaatcagctatactctgctagaggctgggaatggtcagaccacaaaccattaaactttctcaactgggatccagacaggcccagtgcacctactataggtggctccagctgtgcaagaatggatgctgagtctggtctgtggcagagcttttcctgtgaagctcaactgccctatgtctgcaggaaaccattaaataatacagtggagttaacagatgtctggacatactcagatacccgctgtgatgcaggctggctgccaaataatggattttgctatctgctggtaaatgaaagtaattcctgggataaggcacatgcgaaatgcaaagccttcagtagtgacctaatcagcattcattctctagcagatgtggaggtggttgtcacaaaactccataatgaggatatcaaagaagaagtgtggataggccttaagaacataaacataccaactttatttcagtggtcagatggtactgaagttactctaacatattgggatgagaatgagccaaatgttccctacaataagacgcccaactgtgtttcctacttaggagagctaggtcagtggaaagtccaatcatgtgaggagaaactaaaatatgtatgcaagagaaagggagaaaaactgaatgacgcaagttctgataagatgtgtcctccagatgagggctggaagagacatggagaaacctgttacaagatttatgaggatgaggtcccttttggaacaaactgcaatctgactatcactagcagatttgagcaagaatacctaaatgatttgatgaaaaagtatgataaatctctaagaaaatacttctggactggcctgagagatgtagattcttgtggagagtataactgggcaactgttggtggaagaaggcgggctgtaaccttttccaactggaattttcttgagccagcttccccgggcggctgcgtggctatgtctactggaaagtctgttggaaagtgggaggtgaaggactgcagaagcttcaaagcactttcaatttgcaagaaaatgagtggaccccttgggcctgaagaagcatcccctaagcctgatgacccctgtcctgaaggctggcagagtttccccgcaagtctttcttgttataaggtattccatgcagaaagaattgtaagaaagaggaactgggaagaagctgaacgattctgccaagcccttggagcacacctttctagcttcagccatgtggatgaaataaaggaatttcttcactttttaacggaccagttcagtggccagcattggctgtggattggtttgaataaaaggagcccagatttacaaggatcctggcaatggagtgatcgtacaccagtgtctactattatcatgccaaatgagtttcagcaggattatgacatcagagactgtgctgctgtcaaggtatttcataggccatggcgaagaggctggcatttctatgatgatagagaatttatttatttgaggccttttgcttgtgatacaaaacttgaatgggtgtgccaaattccaaaaggccgtactccaaaaacaccagactggtacaatccagaccgtgctggaattcatggacctccacttataattgaaggaagtgaatattggtttgttgctgatcttcacctaaactatgaagaagccgtcctgtactgtgccagcaatcacagctttcttgcgactataacatcttttgtgggactaaaagccatcaaaaacaaaatagcaaatatatctggtgatggacagaagtggtggataagaattagcgagtggccaatagatgatcattttacatactcacgatatccatggcaccgctttcctgtgacatttggagaggaatgcttgtacatgtctgccaagacttggcttatcgacttaggtaaaccaacagactgtagtaccaagttgcccttcatctgtgaaaaatataatgtttcttcgttagagaaatacagcccagattctgcagctaaagtgcaatgttctgagcaatggattccttttcagaataagtgttttctaaagatcaaacccgtgtctctcacattttctcaagcaagcgatacctgtcactcctatggtggcacccttccttcagtgttgagccagattgaacaagactttattacatccttgcttccggatatggaagctactttatggattggtttgcgctggactgcctatgaaaagataaacaaatggacagataacagagagctgacgtacagtaactttcacccattattggttagtgggaggctgagaataccagaaaatttttttgaggaagagtctcgctaccactgtgccctaatactcaacctccaaaaatcaccgtttactgggacgtggaattttacatcctgcagtgaacgccactttgtgtctctctgtcagaaatattcagaagttaaaagcagacagacgttgcagaatgcttcagaaactgtaaagtatctaaataatctgtacaaaataatcccaaagactctgacttggcacagtgctaaaagggagtgtctgaaaagtaacatgcagctggtgagcatcacggacccttaccagcaggcattcctcagtgtgcaggcgctccttcacaactcttccttatggatcggactcttcagtcaagatgatgaactcaactttggttggtcagatgggaaacgtcttcattttagtcgctgggctgaaactaatgggcaactcgaagactgtgtagtattagacactgatggattctggaaaacagttgattgcaatgacaatcaaccaggtgctatttgctactattcaggaaatgagactgaaaaagaggtcaaaccagttgacagtgttaaatgtccatctcctgttctaaatactccgtggataccatttcagaactgttgctacaatttcataataacaaagaataggcatatggcaacaacacaggatgaagttcatactaaatgccagaaactgaatccaaaatcacatattctgagtattcgagatgaaaaggagaataactttgttcttgagcaactgctgtacttcaattatatggcttcatgggtcatgttaggaataacttatagaaataattctcttatgtggtttgataagaccccactgtcatatacacattggagagcaggaagaccaactataaaaaatgagaagtttttggctggtttaagtactgacggcttctgggatattcaaacctttaaagttattgaagaagcagtttattttcaccagcacagcattcttgcttgtaaaattgaaatggttgactacaaagaagaacataatactacactgccacagtttatgccatatgaagatggtatttacagtgttattcaaaaaaaggtaacatggtatgaagcattaaacatgtgttctcaaagtggaggtcacttggcaagcgttcacaaccaaaatggccagctctttctggaagatattgtaaaacgtgatggatttccactatgggttgggctctcaagtcatgatggaagtgaatcaagttttgaatggtctgatggtagtacatttgactatatcccatggaaaggccaaacatctcctggaaattgtgttctcttggatccaaaaggaacttggaaacatgaaaaatgcaactctgttaaggatggtgctatttgttataaacctacaaaatctaaaaagctgtcccgtcttacatattcatcaagatgtccagcagcaaaagagaatgggtcacggtggatccagtacaagggtcactgttacaagtctgatcaggcattgcacagtttttcagaggccaaaaaattgtgttcaaaacatgatcactctgcaactatcgtttccataaaagatgaagatgagaataaatttgtgagcagactgatgagggaaaataataacattaccatgagagtttggcttggattatctcaacattctgttgaccagtcttggagttggttagatggatcagaagtgacatttgtcaaatgggaaaataaaagtaagagtggtgttggaagatgtagcatgttgatagcttcaaatgaaacttggaaaaaagttgaatgtgaacatggttttggaagagttgtctgcaaagtgcctctggactgtccttcatctacttggattcagttccaagacagttgttacatttttctccaagaagccatcaaagtagaaagcatagaggatgtcagaaatcagtgtactgaccatggagcggacatgataagcatacataatgaagaagaaaatgcttttatactggatactttgaaaaagcaatggaaaggcccagatgatatcctactaggcatgttttatgacacagatgatgcgagtttcaagtggtttgataattcaaatatgacatttgataagtggacagaccaagatgatgatgaggatttagttgacacctgtgcttttctgcacatcaagacaggtgaatggaaaaaaggaaattgtgaagtttcttctgtggaaggaacactatgcaaaacagctatcccatacaaaaggaaatatttatcagataaccacattttaatatcagcattggtgattgctagcacggtaattttgacagttttgggagcaatcatttggttcctgtacaaaaaacattctgattctcgtttcaccacagttttttcaaccgcaccccaatcaccttataatgaagactgtgttttggtagttggagaagaaaatgaatatcctgttcaatttgactaa

SEQ ID NO: 2 - protein, 1873 aa, Homo sapiens [sequence omitted]

SEQ ID NO: 3 - DNA, 5622 bp, Homo sapiens
tactcctgtccgacccgctggggagcggcgggccgccccgaggagtacgacgagaagaccaagaagctagagcgcctcgggagaccggcgcgtcgattactggggaagtggtagcaggtacctttatgcccgttcacgtagttcggtcacataccgacctatcatcgtctgctgacactactttgactcctgttcaataccttcacccacagggtcgtagccgagaaagtaaacgtgagggttttcacggaaccggagctataatggtttagccatttactcgactcttacaagtcgacactgaggtcacggtacgacaccacctttacactcgtggtgagagacatgcctcgacgggccatggccgaccgagacttcctacctgtaccgtgtcgttagagtttacgtagactacagaccttctttcctccgagtctcctttcggaaacactggtcggaatagtactctagatatggtctctacccttgagaataccctctggaacacttaaaggtaagaattaactaccctggaccgtagtactaacgtaagaactacttctagtatcacccggtaccacacggtggtggaatttaatacttatactggctttcaccccgtagacgaatttcggacttttgccaacacttctattaacccttttcttgctcgtcaaaccttcaacgatggttaaattatgagtctgccgagaaagaacctttcttcgaatacaaagtacagtcttagttcctcgactaaatgactcgtagttgtcacgacgacttaattgaatggaatttctttttcttccgtaacgattctaaaagacctaaccaaatttagtcgatatgagacgatctccgacccttaccagtctggtgtttggtaatttgaaagagttgaccctaggtctgtccgggtcacgtggatgatatccaccgaggtcgacacgttcttacctacgactcagaccagacaccgtctcgaaaaggacacttcgagttgacgggatacagacgtcctttggtaatttattatgtcacctcaattgtctacagacctgtatgagtctatgggcgacactacgtccgaccgacggtttattacctaaaacgatagacgaccatttactttcattaaggaccctattccgtgtacgctttacgtttcggaagtcatcactggattagtcgtaagtaagagatcgtctacacctccaccaacagtgttttgaggtattactcctatagtttcttcttcacacctatccggaattcttgtatttgtatggttgaaataaagtcaccagtctaccatgacttcaatgagattgtataaccctactcttactcggtttacaagggatgttattctgcgggttgacacaaaggatgaatcctctcgatccagtcacctttcaggttagtacactcctctttgattttatacatacgttctctttccctctttttgacttactgcgttcaagactattctacacaggaggtctactcccgaccttctctgtacctctttggacaatgttctaaatactcctactccagggaaaaccttgtttgacgttagactgatagtgatcgtctaaactcgttcttatggatttactaaactactttttcatactatttagagattcttttatgaagacctgaccggactctctacatctaagaacacctctcatattgacccgttgacaaccaccttcttccgcccgacattggaaaaggttgaccttaaaagaactcggtcgaaggggcccgccgacgcaccgatacagatgacctttcagacaacctttcaccctccacttcctgacgtcttcgaagtttcgtgaaagttaaacgttcttttactcacctggggaacccggacttcttcgtaggggattcggactactggggacaggacttccgaccgtctcaaaggggcgttcagaaagaacaatattccataaggtacgtctttcttaacattctttctccttgacccttcttcgacttgctaagacggttcgggaacctcgtgtggaaagatcgaagtcggtacacctactttatttccttaaagaagtgaaaaattgcctggtcaagtcaccggtcgtaaccgacacctaaccaaacttattttcctcgggtctaaatgttcctaggaccgttacctcactagcatgtggtcacagatgataatagtacggtttactcaaagtcgtcctaatactgtagtctctgacacgacgacagttccataaagtatccggtaccgcttctccgaccgtaaagatactactatctcttaaataaataaactccggaaaacgaacactatgttttgaacttacccacacggtttaaggttttccggcatgaggtttttgtggtctgaccatgttaggtctggcacgaccttaagtacctggaggtgaatattaacttccttcacttataaccaaacaacgactagaagtggatttgatacttcttcggcaggacatgacacggtcgttagtgtcgaaagaacgctgatattgtagaaaacaccctgattttcggtagtttttgttttatcgtttatatagaccactacctgtcttcaccacctattcttaatcgctcaccggttatctactagtaaaatgtatgagtgctataggtaccgtggcgaaaggacactgtaaacctctccttacgaacatgtacagacggttctgaaccgaatagctgaatccatttggttgtctgacatcatggttcaacgggaagtagacactttttatattacaaagaagcaatctctttatgtcgggtctaagacgtcgatttcacgttacaagactcgttacctaaggaaaagtcttattcacaaaagatttctagtttgggcacagagagtgtaaaagagttcgttcgctatggacagtgaggataccaccgtgggaaggaagtcacaactcggtctaacttgttctgaaataatgtaggaacgaaggcctataccttcgatgaaatacctaaccaaacgcgacctgacggatacttttctatttgtttacctgtctattgtctctcgactgcatgtcattgaaagtgggtaataaccaatcaccctccgactcttatggtcttttaaaaaaactccttctcagagcgatggtgacacgggattatgagttggaggtttttagtggcaaatgaccctgcaccttaaaatgtaggacgtcacttgcggtgaaacacagagagacagtctttataagtcttcaattttcgtctgtctgcaacgtcttacgaagtctttgacatttcatagatttattagacatgttttattagggtttctgagactgaaccgtgtcacgattttccctcacagacttttcattgtacgtcgaccactcgtagtgcctgggaatggtcgtccgtaaggagtcacacgtccgcgaggaagtgttgagaaggaatacctagcctgagaagtcagttctactacttgagttgaaaccaaccagtctaccctttgcagaagtaaaatcagcgacccgactttgattacccgttgagcttctgacacatcataatctgtgactacctaagaccttttgtcaactaacgttactgttagttggtccacgataaacgatgataagtcctttactctgactttttctccagtttggtcaactgtcacaatttacaggtagaggacaagatttatgaggcacctatggtaaagtcttgacaacgatgttaaagtattattgtttcttatccgtataccgttgttgtgtcctacttcaagtatgatttacggtctttgacttaggttttagtgtataagactcataagctctacttttcctcttattgaaacaagaactcgttgacgacatgaagttaatataccgaagtacccagtacaatccttattgaatatctttattaagagaatacaccaaactattctggggtgacagtatatgtgtaacctctcgtccttctggttgatattttttactcttcaaaaaccgaccaaattcatgactgccgaagaccctataagtttggaaatttcaataacttcttcgtcaaataaaagtggtcgtgtcgtaagaacgaacattttaactttaccaactgatgtttcttcttgtattatgatgtgacggtgtcaaatacggtatacttctaccataaatgtcacaataagtttttttccattgtaccatacttcgtaatttgtacacaagagtttcacctccagtgaaccgttcgcaagtgttggttttaccggtcgagaaagaccttctataacattttgcactacctaaaggtgatacccaacccgagagttcagtactaccttcacttagttcaaaacttaccagactaccatcatgtaaactgatatagggtacctttccggtttgtagaggacctttaacacaagagaacctaggttttccttgaacctttgtactttttacgttgagacaattcctaccacgataaacaatatttggatgttttagatttttcgacagggcagaatgtataagtagttctacaggtcgtcgttttctcttacccagtgccacctaggtcatgttcccagtgacaatgttcagactagtccgtaacgtgtcaaaaagtctccggttttttaacacaagttttgtactagtgagacgttgatagcaaaggtattttctacttctactcttatttaaacactcgtctgactactcccttttattattgtaatggtactctcaaaccgaacctaatagagttgtaagacaactggtcagaacctcaaccaatctacctagtcttcactgtaaacagtttacccttttattttcattctcaccacaaccttctacatcgtacaactatcgaagtttactttgaaccttttttcaacttacacttgtaccaaaaccttctcaacagacgtttcacggagacctgacaggaagtagatgaacctaagtcaaggttctgtcaacaatgtaaaaagaggttcttcggtagtttcatctttcgtatctcctacagtctttagtcacatgactggtacctcgcctgtactattcgtatgtattacttcttcttttacgaaaatatgacctatgaaactttttcgttacctttccgggtctactataggatgatccgtacaaaatactgtgtctactacgctcaaagttcaccaaactattaagtttatactgtaaactattcacctgtctggttctactactactcctaaatcaactgtggacacgaaaagacgtgtagttctgtccacttaccttttttcctttaacacttcaaagaagacaccttccttgtgatacgttttgtcgatagggtatgttttcctttataaatagtctattggtgtaaaattatagtcgtaaccactaacgatcgtgccattaaaactgtcaaaaccctcgttagtaaaccaaggacatgttttttgtaagactaagagcaaagtggtgtcaaaaaagttggcgtggggttagtggaatattacttctgacacaaaaccatcaacctcttcttttacttataggacaagttaaactgatt

SEQ ID NO: 4 - DNA, 5454 bp, Homo sapiens
atgaggacaggctgggcgacccctcgccgcccggcggggctcctcatgctgctcttctggttcttcgatctcgcggagccctctggccgcgcagctaatgaccccttcaccatcgtccatggaaatacgggcaagtgcatcaagccagtgtatggctggatagtagcagacgactgtgatgaaactgaggacaagttatggaagtgggtgtcccagcatcggctctttcatttgcactcccaaaagtgccttggcctcgatattaccaaatcggtaaatgagctgagaatgttcagctgtgactccagtgccatgctgtggtggaaatgtgagcaccactctctgtacggagctgcccggtaccggctggctctgaaggatggacatggcacagcaatctcaaatgcatctgatgtctggaagaaaggaggctcagaggaaagcctttgtgaccagccttatcatgagatctataccagagatgggaactcttatgggagaccttgtgaatttccattcttaattgatgggacctggcatcatgattgcattcttgatgaagatcatagtgggccatggtgtgccaccaccttaaattatgaatatgaccgaaagtggggcatctgcttaaagcctgaaaacggttgtgaagataattgggaaaagaacgagcagtttggaagttgctaccaatttaatactcagacggctctttcttggaaagaagcttatgtttcatgtcagaatcaaggagctgatttactgagcatcaacagtgctgctgaattaacttaccttaaagaaaaagaaggcattgctaagattttctggattggtttaaatcagctatactctgctagaggctgggaatggtcagaccacaaaccattaaactttctcaactgggatccagacaggcccagtgcacctactataggtggctccagctgtgcaagaatggatgctgagtctggtctgtggcagagcttttcctgtgaagctcaactgccctatgtctgcaggaaaccattaaataatacagtggagttaacagatgtctggacatactcagatacccgctgtgatgcaggctggctgccaaataatggattttgctatctgctggtaaatgaaagtaattcctgggataaggcacatgcgaaatgcaaagccttcagtagtgacctaatcagcattcattctctagcagatgtggaggtggttgtcacaaaactccataatgaggatatcaaagaagaagtgtggataggccttaagaacataaacataccaactttatttcagtggtcagatggtactgaagttactctaacatattgggatgagaatgagccaaatgttccctacaataagacgcccaactgtgtttcctacttaggagagctaggtcagtggaaagtccaatcatgtgaggagaaactaaaatatgtatgcaagagaaagggagaaaaactgaatgacgcaagttctgataagatgtgtcctccagatgagggctggaagagacatggagaaacctgttacaagatttatgaggatgaggtcccttttggaacaaactgcaatctgactatcactagcagatttgagcaagaatacctaaatgatttgatgaaaaagtatgataaatctctaagaaaatacttctggactggcctgagagatgtagattcttgtggagagtataactgggcaactgttggtggaagaaggcgggctgtaaccttttccaactggaattttcttgagccagcttccccgggcggctgcgtggctatgtctactggaaagtctgttggaaagtgggaggtgaaggactgcagaagcttcaaagcactttcaatttgcaagaaaatgagtggaccccttgggcctgaagaagcatcccctaagcctgatgacccctgtcctgaaggctggcagagtttccccgcaagtctttcttgttataaggtattccatgcagaaagaattgtaagaaagaggaactgggaagaagctgaacgattctgccaagcccttggagcacacctttctagcttcagccatgtggatgaaataaaggaatttcttcactttttaacggaccagttcagtggccagcattggctgtggattggtttgaataaaaggagcccagatttacaaggatcctggcaatggagtgatcgtacaccagtgtctactattatcatgccaaatgagtttcagcaggattatgacatcagagactgtgctgctgtcaaggtatttcataggccatggcgaagaggctggcatttctatgatgatagagaatttatttatttgaggccttttgcttgtgatacaaaacttgaatgggtgtgccaaattccaaaaggccgtactccaaaaacaccagactggtacaatccagaccgtgctggaattcatggacctccacttataattgaaggaagtgaatattggtttgttgctgatcttcacctaaactatgaagaagccgtcctgtactgtgccagcaatcacagctttcttgcgactataacatcttttgtgggactaaaagccatcaaaaacaaaatagcaaatatatctggtgatggacagaagtggtggataagaattagcgagtggccaatagatgatcattttacatactcacgatatccatggcaccgctttcctgtgacatttggagaggaatgcttgtacatgtctgccaagacttggcttatcgacttaggtaaaccaacagactgtagtaccaagttgcccttcatctgtgaaaaatataatgtttcttcgttagagaaatacagcccagattctgcagctaaagtgcaatgttctgagcaatggattccttttcagaataagtgttttctaaagatcaaacccgtgtctctcacattttctcaagcaagcgatacctgtcactcctatggtggcacccttccttcagtgttgagccagattgaacaagactttattacatccttgcttccggatatggaagctactttatggattggtttgcgctggactgcctatgaaaagataaacaaatggacagataacagagagctgacgtacagtaactttcacccattattggttagtgggaggctgagaataccagaaaatttttttgaggaagagtctcgctaccactgtgccctaatactcaacctccaaaaatcaccgtttactgggacgtggaattttacatcctgcagtgaacgccactttgtgtctctctgtcagaaatattcagaagttaaaagcagacagacgttgcagaatgcttcagaaactgtaaagtatctaaataatctgtacaaaataatcccaaagactctgacttggcacagtgctaaaagggagtgtctgaaaagtaacatgcagctggtgagcatcacggacccttaccagcaggcattcctcagtgtgcaggcgctccttcacaactcttccttatggatcggactcttcagtcaagatgatgaactcaactttggttggtcagatgggaaacgtcttcattttagtcgctgggctgaaactaatgggcaactcgaagactgtgtagtattagacactgatggattctggaaaacagttgattgcaatgacaatcaaccaggtgctatttgctactattcaggaaatgagactgaaaaagaggtcaaaccagttgacagtgttaaatgtccatctcctgttctaaatactccgtggataccatttcagaactgttgctacaatttcataataacaaagaataggcatatggcaacaacacaggatgaagttcatactaaatgccagaaactgaatccaaaatcacatattctgagtattcgagatgaaaaggagaataactttgttcttgagcaactgctgtacttcaattatatggcttcatgggtcatgttaggaataacttatagaaataattctcttatgtggtttgataagaccccactgtcatatacacattggagagcaggaagaccaactataaaaaatgagaagtttttggctggtttaagtactgacggcttctgggatattcaaacctttaaagttattgaagaagcagtttattttcaccagcacagcattcttgcttgtaaaattgaaatggttgactacaaagaagaacataatactacactgccacagtttatgccatatgaagatggtatttacagtgttattcaaaaaaaggtaacatggtatgaagcattaaacatgtgttctcaaagtggaggtcacttggcaagcgttcacaaccaaaatggccagctctttctggaagatattgtaaaacgtgatggatttccactatgggttgggctctcaagtcatgatggaagtgaatcaagttttgaatggtctgatggtagtacatttgactatatcccatggaaaggccaaacatctcctggaaattgtgttctcttggatccaaaaggaacttggaaacatgaaaaatgcaactctgttaaggatggtgctatttgttataaacctacaaaatctaaaaagctgtcccgtcttacatattcatcaagatgtccagcagcaaaagagaatgggtcacggtggatccagtacaagggtcactgttacaagtctgatcaggcattgcacagtttttcagaggccaaaaaattgtgttcaaaacatgatcactctgcaactatcgtttccataaaagatgaagatgagaataaatttgtgagcagactgatgagggaaaataataacattaccatgagagtttggcttggattatctcaacattctgttgactgtccttcatctacttggattcagttccaagacagttgttacatttttctccaagaagccatcaaagtagaaagcatagaggatgtcagaaatcagtgtactgaccatggagcggacatgataagcatacataatgaagaagaaaatgcttttatactggatactttgaaaaagcaatggaaaggcccagatgatatcctactaggcatgttttatgacacagatgatgcgagtttcaagtggtttgataattcaaatatgacatttgataagtggacagaccaagatgatgatgaggatttagttgacacctgtgcttttctgcacatcaagacaggtgaatggaaaaaaggaaattgtgaagtttcttctgtggaaggaacactatgcaaaacagctatcccatacaaaaggaaatatttatcagataaccacattttaatatcagcattggtgattgctagcacggtaattttgacagttttgggagcaatcatttggttcctgtacaaaaaacattctgattctcgtttcaccacagttttttcaaccgcaccccaatcaccttataatgaagactgtgttttggtagttggagaagaaaatgaatatcctgttcaatttgactaa

SEQ ID NO: 5 - protein, 1817 aa, Homo sapiens [sequence omitted]

SEQ ID NO: 6 - DNA, 5454 bp, Homo sapiens
tactcctgtccgacccgctggggagcggcgggccgccccgaggagtacgacgagaagaccaagaagctagagcgcctcgggagaccggcgcgtcgattactggggaagtggtagcaggtacctttatgcccgttcacgtagttcggtcacataccgacctatcatcgtctgctgacactactttgactcctgttcaataccttcacccacagggtcgtagccgagaaagtaaacgtgagggttttcacggaaccggagctataatggtttagccatttactcgactcttacaagtcgacactgaggtcacggtacgacaccacctttacactcgtggtgagagacatgcctcgacgggccatggccgaccgagacttcctacctgtaccgtgtcgttagagtttacgtagactacagaccttctttcctccgagtctcctttcggaaacactggtcggaatagtactctagatatggtctctacccttgagaataccctctggaacacttaaaggtaagaattaactaccctggaccgtagtactaacgtaagaactacttctagtatcacccggtaccacacggtggtggaatttaatacttatactggctttcaccccgtagacgaatttcggacttttgccaacacttctattaacccttttcttgctcgtcaaaccttcaacgatggttaaattatgagtctgccgagaaagaacctttcttcgaatacaaagtacagtcttagttcctcgactaaatgactcgtagttgtcacgacgacttaattgaatggaatttctttttcttccgtaacgattctaaaagacctaaccaaatttagtcgatatgagacgatctccgacccttaccagtctggtgtttggtaatttgaaagagttgaccctaggtctgtccgggtcacgtggatgatatccaccgaggtcgacacgttcttacctacgactcagaccagacaccgtctcgaaaaggacacttcgagttgacgggatacagacgtcctttggtaatttattatgtcacctcaattgtctacagacctgtatgagtctatgggcgacactacgtccgaccgacggtttattacctaaaacgatagacgaccatttactttcattaaggaccctattccgtgtacgctttacgtttcggaagtcatcactggattagtcgtaagtaagagatcgtctacacctccaccaacagtgttttgaggtattactcctatagtttcttcttcacacctatccggaattcttgtatttgtatggttgaaataaagtcaccagtctaccatgacttcaatgagattgtataaccctactcttactcggtttacaagggatgttattctgcgggttgacacaaaggatgaatcctctcgatccagtcacctttcaggttagtacactcctctttgattttatacatacgttctctttccctctttttgacttactgcgttcaagactattctacacaggaggtctactcccgaccttctctgtacctctttggacaatgttctaaatactcctactccagggaaaaccttgtttgacgttagactgatagtgatcgtctaaactcgttcttatggatttactaaactactttttcatactatttagagattcttttatgaagacctgaccggactctctacatctaagaacacctctcatattgacccgttgacaaccaccttcttccgcccgacattggaaaaggttgaccttaaaagaactcggtcgaaggggcccgccgacgcaccgatacagatgacctttcagacaacctttcaccctccacttcctgacgtcttcgaagtttcgtgaaagttaaacgttcttttactcacctggggaacccggacttcttcgtaggggattcggactactggggacaggacttccgaccgtctcaaaggggcgttcagaaagaacaatattccataaggtacgtctttcttaacattctttctccttgacccttcttcgacttgctaagacggttcgggaacctcgtgtggaaagatcgaagtcggtacacctactttatttccttaaagaagtgaaaaattgcctggtcaagtcaccggtcgtaaccgacacctaaccaaacttattttcctcgggtctaaatgttcctaggaccgttacctcactagcatgtggtcacagatgataatagtacggtttactcaaagtcgtcctaatactgtagtctctgacacgacgacagttccataaagtatccggtaccgcttctccgaccgtaaagatactactatctcttaaataaataaactccggaaaacgaacactatgttttgaacttacccacacggtttaaggttttccggcatgaggtttttgtggtctgaccatgttaggtctggcacgaccttaagtacctggaggtgaatattaacttccttcacttataaccaaacaacgactagaagtggatttgatacttcttcggcaggacatgacacggtcgttagtgtcgaaagaacgctgatattgtagaaaacaccctgattttcggtagtttttgttttatcgtttatatagaccactacctgtcttcaccacctattcttaatcgctcaccggttatctactagtaaaatgtatgagtgctataggtaccgtggcgaaaggacactgtaaacctctccttacgaacatgtacagacggttctgaaccgaatagctgaatccatttggttgtctgacatcatggttcaacgggaagtagacactttttatattacaaagaagcaatctctttatgtcgggtctaagacgtcgatttcacgttacaagactcgttacctaaggaaaagtcttattcacaaaagatttctagtttgggcacagagagtgtaaaagagttcgttcgctatggacagtgaggataccaccgtgggaaggaagtcacaactcggtctaacttgttctgaaataatgtaggaacgaaggcctataccttcgatgaaatacctaaccaaacgcgacctgacggatacttttctatttgtttacctgtctattgtctctcgactgcatgtcattgaaagtgggtaataaccaatcaccctccgactcttatggtcttttaaaaaaactccttctcagagcgatggtgacacgggattatgagttggaggtttttagtggcaaatgaccctgcaccttaaaatgtaggacgtcacttgcggtgaaacacagagagacagtctttataagtcttcaattttcgtctgtctgcaacgtcttacgaagtctttgacatttcatagatttattagacatgttttattagggtttctgagactgaaccgtgtcacgattttccctcacagacttttcattgtacgtcgaccactcgtagtgcctgggaatggtcgtccgtaaggagtcacacgtccgcgaggaagtgttgagaaggaatacctagcctgagaagtcagttctactacttgagttgaaaccaaccagtctaccctttgcagaagtaaaatcagcgacccgactttgattacccgttgagcttctgacacatcataatctgtgactacctaagaccttttgtcaactaacgttactgttagttggtccacgataaacgatgataagtcctttactctgactttttctccagtttggtcaactgtcacaatttacaggtagaggacaagatttatgaggcacctatggtaaagtcttgacaacgatgttaaagtattattgtttcttatccgtataccgttgttgtgtcctacttcaagtatgatttacggtctttgacttaggttttagtgtataagactcataagctctacttttcctcttattgaaacaagaactcgttgacgacatgaagttaatataccgaagtacccagtacaatccttattgaatatctttattaagagaatacaccaaactattctggggtgacagtatatgtgtaacctctcgtccttctggttgatattttttactcttcaaaaaccgaccaaattcatgactgccgaagaccctataagtttggaaatttcaataacttcttcgtcaaataaaagtggtcgtgtcgtaagaacgaacattttaactttaccaactgatgtttcttcttgtattatgatgtgacggtgtcaaatacggtatacttctaccataaatgtcacaataagtttttttccattgtaccatacttcgtaatttgtacacaagagtttcacctccagtgaaccgttcgcaagtgttggttttaccggtcgagaaagaccttctataacattttgcactacctaaaggtgatacccaacccgagagttcagtactaccttcacttagttcaaaacttaccagactaccatcatgtaaactgatatagggtacctttccggtttgtagaggacctttaacacaagagaacctaggttttccttgaacctttgtactttttacgttgagacaattcctaccacgataaacaatatttggatgttttagatttttcgacagggcagaatgtataagtagttctacaggtcgtcgttttctcttacccagtgccacctaggtcatgttcccagtgacaatgttcagactagtccgtaacgtgtcaaaaagtctccggttttttaacacaagttttgtactagtgagacgttgatagcaaaggtattttctacttctactcttatttaaacactcgtctgactactcccttttattattgtaatggtactctcaaaccgaacctaatagagttgtaagacaactgacaggaagtagatgaacctaagtcaaggttctgtcaacaatgtaaaaagaggttcttcggtagtttcatctttcgtatctcctacagtctttagtcacatgactggtacctcgcctgtactattcgtatgtattacttcttcttttacgaaaatatgacctatgaaactttttcgttacctttccgggtctactataggatgatccgtacaaaatactgtgtctactacgctcaaagttcaccaaactattaagtttatactgtaaactattcacctgtctggttctactactactcctaaatcaactgtggacacgaaaagacgtgtagttctgtccacttaccttttttcctttaacacttcaaagaagacaccttccttgtgatacgttttgtcgatagggtatgttttcctttataaatagtctattggtgtaaaattatagtcgtaaccactaacgatcgtgccattaaaactgtcaaaaccctcgttagtaaaccaaggacatgttttttgtaagactaagagcaaagtggtgtcaaaaaagttggcgtggggttagtggaatattacttctgacacaaaaccatcaacctcttcttttacttataggacaagttaaactgatt

SEQ ID NO: 7 - DNA, 3740 bp, Homo sapiens
ctctccggccgcgcagccgctgccgcccacccgcacccgccgtcatgctccgggccgcgctgcccgcgctcctgctgccgttgctgggcctcgccgctgctgccgtcgcggactgtccttcatctacttggattcagttccaagacagttgttacatttttctccaagaagccatcaaagtagaaagcatagaggatgtcagaaatcagtgtactgaccatggagcggacatgataagcatacataatgaagaagaaaatgcttttatactggatactttgaaaaagcaatggaaaggcccagatgatatcctactaggcatgttttatgacacagatgatgcgagtttcaagtggtttgataattcaaatatgacatttgataagtggacagaccaagatgatgatgaggatttagttgacacctgtgcttttctgcacatcaagacaggtgaatggaaaaaaggaaattgtgaagtttcttctgtggaaggaacactatgcaaaacagctatcccatacaaaaggaaatatttatcagataaccacattttaatatcagcattggtgattgctagcacggtaattttgacagttttgggagcaatcatttggttcctgtacaaaaaacattctgattctcgtttcaccacagttttttcaaccgcaccccaatcaccttataatgaagactgtgttttggtagttggagaagaaaatgaatatcctgttcaatttgactaagtttttggtaatcttgcactaagacatcaacaaatgccctggcagagataacttgggaaagattttaatataaaacttgacattggatattagagctttaatggtattccttattccagtaacatttttatgtactcatctgctgtgaaaagtctttaggttcattaaaaaaacaggttttagaaatgatcttagatctaatatagtgattttaagcatcccgtcaaaggcagaatctgtcacttgaatgaaggaaagcttaaagcccaagcagataaaaataaaagcccagcctatttgtcttgcctgctgtatcttccctatttagttgacccactttagtttatatgtttattagtaaacatgaaatggggaataagtgattttaagtacatcccatacatttaaatatctttgataattgttatttttttggcagataattcctctagaatgtgtatctttttatgatttagatgaagaaaattttacaacttttaacaccccacaccaattttagtttcattacttttacacacaccattttatcacaaatgactcaagttttaatgaatgtttataaattatttgaaacaaaatatgatcgctgtgtccaggatggcatagagaaagctggcaattaggttaacacttacatattatagtgcccctttaaggatttctctcttgccaccataccttttgtactttcccctatacaagatgtatctcattctcctcaagcatttataaatttttccttcaatgacatgaaaactgtgcaagcaaaaaccgaagaaaaacacttaagtacaactgtagtgacagtgatcaaagttttcagtgcatttattgtacattttaagaaaaaggtgaaaatcatttggggagtaaaaaaatgaaaaagctgaaacgagtaattttcctcaccatcaataaaccaaaaacaggaaagataaagaatgtataaatttcacgtaaattagtcacgtatcacttatcaatggggatacgttctaagaaatgcatagttagggaatcttgtgtgaaaatcagcttgtatttacacaaacccagatggtagagcctattttgtcccaaacctacacagcatgttactgtgctgaatactgcagacaattgtaacacaatatttgtgtatctaaatatagaaaaggtacagtaaaaatatggtctactaaggaaacactgttctatatgtggtccattactgactgaagtatactgtctagaagtctgaggctcaaagaaaagtaatccctcttctgaatccacaccccatcaattatcttactttcttctggggagatagatagatatactatctcactagcttgactaatggcaacaaagttccagcttgtgtagtctctttttattgaccacatgaatcgaaaacactcatcacaattaatggcactatcattaatgagacatgagtaactaaaaagtgatagaaaactattacagtgcggctacatggtactgaaaatgcaggcattacaccagctgttacacaagcacaagcatgctctgtaagagctttacatttctgagattttgtatagtgattgagatgtctattttattattgatagactattactaatgtcaatattgaacactaccctggaattcctgcctggttttcctacccaaattgtaccactccttgaagaactacaggcacagtaaaaaaatatggcgtattatgtgaactaaaagagttctaaaggagttcttaaaggagtggtagaatttgggtaggaaagtgattaagtccaacttaaaaccaacagtctcaaacgtctacaactacaatgtccaatgagccactagccacatgaggctatttaagtaaatttagtttaaaatccagttttcgaattacattagccacattgtcaagtgttcaaatcacaggtggttagtggctactgtactgggcaacatacattatagaacattttcattataggaagttttattgggcagtgctgctcttaaatcctaccttccactcaactcccatacaactttcttttgtacattttgatactttctacctaatggcagctcttccaaaatagctgctttaaactctgatttaattttcaatatttggtttcatttttcaacaggccaagaggcctctggtaatgaagtgctatatatatatatatatgacggagtctcactgtgctgcccaggctacagtgcagtggctcgatcttggctctctccaatctccgccttgcaggttttcaagcaattctcctgcctcagcctccttagtagctgggaccacagacatctgtcaccacacccagctaactttttgtatttttggtagagacggggtttcgccatattgactgggctggtctcaaactcctgacctcaagtgatccacccaccttggtctcccaaagtgctgggattacatgcgtgagccaccacacttggcctacattttttctttatataccagaacatctataacaggcaccttatctactcattagtgaagagataattggattacacaggcaggcttgtttactacatccagaatgtagaaactgctttcttcaacatcttggttctagctagtaataacaatataattctttggcagatattcagaataacattttaaactacattttcttagaaaattgcattcttgtagtgagcagtgtatggtctcttttgttcagaatttaaaactgataaccaatgaaagccttttctcttattcctctaccgtcatttacatgataatctgaagctaatatgacaatatttaaatactaagtggtactagggaactacaagaatactgtaaagcttaagccattgttatcactgtcatttagcatttaataacaaaactatacagaattatgtgcataccaatgaatgttttgtaccatctagttaaattttttaaataaagttttatgggttaagcag

SEQ ID NO: 8 - protein, 232 aa, Homo sapiens
MLRAALPALLLPLLGLAAAAVADCPSSTWIQFQDSCYIFLQEAIKVESIEDVRNQCTDHGADMISIHNEEENAFILDTLKKQWKGPDDILLGMFYDTDDASFKWFDNSNMTFDKWTDQDDDEDLVDTCAFLHIKTGEWKKGNCEVSSVEGTLCKTAIPYKRKYLSDNHILISALVIASTVILTVLGAIIWFLYKKHSDSRFTTVFSTAPQSPYNEDCVLVVGEENEYPVQFD

SEQ ID NO: 9 - DNA, 32460 bp, Homo sapiens
attttttttaactgggtcatttgttttcacgttgttgagtttagacttgattttatagaactcttttgccaatgacatgaggcttaaacaagcacccttgcttgaccctggctagtgttgtccagaggccctcatgggtgaagtataattgcacagtttatgcatacattgtagcaaacatagcttaaagactcttcatccaggagctggtaacagagaaaagttgtctaagagacatagtccactgaaaggggtctttgaaaaggaggattctggccagtagatccaaagaagaaggctggcaaacgccaccttaaccaactgatccaagttaacatcagtgatgagacatcgacatcttgtccttctcgagcacgtacatcacttctggggaactctttcccaaaaaagcgcaacctgaatccaattagaaggaaatgtcagacaaaatgaaactgagggacattctacaaaacgactggccagtactttttaaatgtggtttgatcgtgaaagaaaaagaatgacagagaaccgtcctagattaaagggagactaaggagacacgacatgcaatgtatgatcctggattgaatcttggaacagaaaaggacccttagaggggcaattaatgaaatgtggtaagtgctgtagattagctaatcgcattatcccagtgcgagtgccctgtgtttgatcactgcactggcgttatatacaatgtcaatatttagagaagctggctgaagggttacggaaatctcttgtgctgtttttgcaacattttcgaaagtcctaaattatttcaattcaaatgaaaagtttaaaaaacaaaaattagaagttccagaaggccgcgcgccagccgctcctgcgggacgggacacccgggttctcctggtgggagcccccagtgccgttcacgttccgcccgggggggggattaaactcgcacgcgagaagcaccgcccccgctcccgccccgccccgctcctctccggccgcgcagccgctgccgcccacccgcacccgccgtcatgctccgggccgcgctgcccgcgctcctgctgccgttgctgggcctcgccgctgctgccgtcgcgggtaagcccttacgtagtccctcgccgggaccgtgcgcgaccgccttcgcccccttcccaacgcacgctcttcgtccccgcgcacccgagggcggcccgcagacgcaacacccggccggccatcccgcccttccctgcacgcccgtcccccgtgggtcctggctccgggtcacctctcacccgcctgccctcggggaggggaggtggccgagaataagggagggctctgtcttcctcggagtccacatcctcaccgcagaccccactccgcggggagggaacccccaaattaggccagttggccggagaactgagggacttggagtcgcacgacgggcgccgtttcagggcaatttcgggctgaaatgagaagcggggacgttggtggcgatttcccctgctggtgcgcggccggagtggggttgctgggatgggggtgggggccggaggaagtaggccctcttttgcaagcagcgctgtttgtctagttggttggtgttcaagttgtttaaacaggaaaacagttcagccaaataacccctggatggaagaggaacgggaataggcaaagcttggatttcactgaaatcaaggagttttaaagttctagtctgctgttgtgcaagtgacatctgaaaaatcacacacgtgatcattcatttacaaaacgactcgtgaggaaaatgcacaattctattgaccgtggtctttatttttaaaaaatttccatacaagcatgtcaaaaatatgtggatggggagactctggagaacacagacttccaaaaacaccactgactgaataattccaggaattaaagagcaaaataaacaagaactaaatgagtacttgtgtgggcttaaataaagtgcaagagatttaaataaaatgcaagagattccccccccccaccccttgccccagatttcactgcgtttttataataactgcctgctcgaagtctactgacaggaatatttcagtggacctcagtgttggaggcagcagcagctcagaacttggatacaaacccaaggttcctttcttgaaaacttctgtggacctgcatttatgactggttgtgacatctgctgcctatcaaaggggcagaaacaagatgtgcccatgttcacattgttcagactgggaacattaattttgtctaagacaaagctgggctgtctctgaaccctccttctgcacaccctcattttgcgagccagtaacatctcaactctcatgtaaaccaccctctgcgaggctgtgcatttgtactttaggctagtcgaattttcttgtcagatttttctttcttgtcagacttttaaagaaaatcagtttctagattttggtatgtctcttcttcagtgaagctgttttgaccagcaatagagggcaaatttccctttggaaatttttgtgcatttcctttgataagtccagtgtggatcaataggcttttcaagagctttagaaaagtgcatgatgaataaattaatgttaattaatcagctcctcccagtcaggaagctttaaggattaatttggaaatgagtgtgagctttgacctagctagttaaccaacttatctgcacttcagtaaaacagagataatacttactcatggggctattgggagcattaagtgggaactccacgtctagtccctattacaggcgtggttcatcttggtttccttccctttattctcttcatacaaaatgaagggtaattgttgcaaccagaaaacgtatgaataccaccttatgtatattggatgtttatggttactgaacacattcatatgtatgctaatgttatagggctgaaaaactaagtgtgtttttcataatactttacaaatctcccatccaagcaagatcaggggtcatatttggcttagaactaagtcaagaaagagtttgttgctgaataccaagatcttaatagaaaagctcttatgatgttgcataataaatatgggtattgcatataaatgtgatgttgaaacggaaatcattgttattgtctgtcattctggaggttattagtgaagtgatcttaaccttgttcttagctattattttgaaaatcacagtggacaaaacattctttaaattcctgagtgaaaatccatggcatcgctttaaaaagtttttccgtaagggtgcttaagcacataatagatgatcagtatgtatttatttggtcagtggtttcctatgcctggcatgagctgagtgaacaagcatgttccgagtaagtcctcattctgtgatcatcatgtggatcagaatctagggattttgaattgccatgtcctacaagccttagatgaggtgcctctgccttcttccctgatgtcatctcttaccacccttcctgccattcactctgtgtatccacagagtaaacagtatctgggtactccaacacctctggtccttctgtgcacaccaccttcccccagtccttggcatggctgcccccacctccaccattcagatctctgttcatatgtcaccttctccgaagcctccctgaacacccacattctctgtcacatgtccatgttttatcttttccaagcctttctgaatgcattgtgtttattcatctgtcttgcttgttgcttctcttaccaggggaaagaagctccataacagagattttaccatcttgtggatttttgttttgttttgatttgtgtctctgcactttattctaagttataataaaaggatgaaaaaatttaggtgagttatttaaaaagtaggtaggataagtggattgcaatttttttcaaattatattacctctgctatgcactcatttcataatgaattcaaagccttaaatcatcttaatctattgctgccaccttttctttctttctttttttttttttccgagatggagtctcactctgtcacccaggctggagtgcggtggcgcaatctcggctcacagcaacctctaccttctgggttcaagcaattctcctgcctcagcctcccgagtagctgggattacaggcatgcgccaccatgcctggctaacttttgtatttttagtagagatggggtttcaccatgttggccaggctggtctcaaactcctggcctcgtgatctgcccacctcggcctcccaaagtgctgggattacaggtgtgagccaccgtgcccggcctgatgccaccttttctgactcttgtgtatggcacagttatgtttcaggggaaattatctcagattaaatattagagtattttaaaataaattttgagctgggcgtggtggctcacgcctgtaatcccagcactttgggaggccgaggcgggtggatcacgaggtcaggagatcgagaccatcctggctaacacggtgaaaccctgtctctactaaaaatacaaaaaattagccgggcgtggtggcaggcacctgtggtcccagctactcaggaggctgaggcaggagaatggcgtgaacccaggagacagagcttgcagtgagctgagatcgcgccactgcactccagcctgggcgacagagcgagactcagtctcaaaataaataaatgaataaataaataaataaataaataaataaattaattaattttgaaatgttaatgtttatttttcttccaatgaaaaagtgaaatttcaattaggtattatttcaggacttcttttactgttgagaatcttttaacaatttgcttatgtgttgaaaactagttttgcagcaaggttactttccataatttttattgtgcatatcaagtatttcattccttctgtgggttatttagtgtttttttttttattattttattttattttattttattttattttattgttttgagacagggcctcactctgtcacccaggctagagtgcagtggtgcaatcatggctcactacagccttgacctgctgggctcaagtgatcctcccacctcagccccctgagtagctgggactacaggtgcatgccaccatgccctgctaattttttttttttttttttgtagaggtggggtctcactttgttgcccaggctagtcccaaactcctgagctcaagtgatcctcctacctcagccttccaaagctctgggattacaggtgtgaactgccacgcctggcctattttagattttgagagcaaatcttgagaccaaatctttaaaaaatagaccagtgatgcccaaagagaaaggtacagattaacaatggcacaccccttctatgaatatacattataacagcccttctgaagagggaagtgatgggacaatatggagatggatgtcttgaaagctagattgggaagagtgatatggagaggggatgaggtgtcagagtgcctcaatttatttgggattcagagagatcttgaagctcagagtacagccacagaatctctggattagtcagggacttggaacagccactccctcctccatacattataaatgtaataaatagcccctcttcttcatctgtctgtgccacagttgcccttgcggaagttatgtgactacaactcatatcttaatgggagaggagcaaaggtcttattattgacaatgaaaatgaaaaagaagtctttcccttttcctttgttaacatttagaacaagtatgtcccaagaatcacttcctcatgctgtgcctgttttttttttctttttttaaaaaaaagagagacagagaacttataaaggaacagagagttctattagatttgactggtaatgcaaagtattcctgtggatatgacattatcttcttttagcacatgataattttaggcagaggatctatatagaaacttttggagttatgctaagtacagcttttaaaatattcatgaggctcctgaatttctgtacgtattaaagaattataaaattataaaatccctaagagtcatttaagtgcattattaataacaacctatcattcaggactgctgtgaagaaataagagtaataggccctttgcagagagcttagtgggcaggtttttaattatattaggattatggcaaagatacttagcgttgaagacgacttagtttagtcttttgttcatgaggtgcatgaacgtcaacaatctcctcacaggagttatccagcctctatttgagcactgttcttctcaaagtaaaaatatatcttttgagtagtgcttaaaacttgtttctggggcacacactctgatcggggatctatagcatgagaaaaggggttccatgttgaattaactggagttactgctaatgctgaaatgagcctaaggagaaggcagttgaaagttttggggaagtgagtcaacagtctgttttctatttttctatacataccctgttaaaaaaaaatttttttttggtcttgtcacatgcagatgctcctcgacttagaatggggctatatcccaataaacttgtgaagtcaaaaaatcgtaagttgtccattattaagttcgggaccatttctgtattgctaacttagttattcagcaatggtctttgagtagctggttatcattatcatcatcatagtcattcccatactgtgcaacacagtagcattgtgtttgctcctccctctgtttgcctgaaatatccatgtgacttctttccctatcttttcagacacacacacacacacacacacacacacacacacacacacacacagagttggttctctgtttctctggagaaccctgactaatacaataaccaataagatagtatggaaataagacagtgtgacaatcaaggctggattgtatgaagcattgcatatccacctttgccctcctttgaatcactcactctgggggaagctacctgccatgtcataaggaccctcaagcacccctgtgtagaagtccacgtggtgaggaactgtggtgtcctgcccacagccagcaccagctcaccacccatatgagtgaggcttcttgaagctgacctttcagctccagttcagtgtttagatggctgcagccctagccagcatcttcactgtaacttcatggagaccccaagccagaatcacccagacaagcaactgcttcagaattcctgacccagagaaactgtatataataagtgtttcttgttttaagctgctaattttttacatagcaatagaccacaatacaccatctaacatattattttatgtatttgtcttccttttgcataactgcgtcaactagaacataaatttccaggagggcaggaatttcttttcctttttgttcactgtggaatctcaagcaccttgcacaattttttaacagcaccccctcagataggtactgttattcccattatacagatgaggaaactgggacacagtaaggttaaataacatgtccacgattgtgtagtcagagccagacttttctccacagtctaatgggcttaaccagagtcctgtattgacccacttctatgcatcccttccagtgttatgttcaagacagaaggatagttattattgagagtagttagatctcagacatggtctctgctgtcgtggcgttaacaatcatttgggaaaacaggatgcatatcagatagccgctatataaagaaggatctaatgagtgctatgtaaacatgtataggccatcagagggtaacacatttccttttccaaagaagaatgactacccatagttcttatgatttgtgccatattatttttaaaaccctagacacaactaattggaataaggataatttccttagtcaaaagctggctactctaggctaggcatcagcccaatatggggcctagtatatgaacgctaagcctatttgactgttgagatagtatcctcagttttcttattggccatgtatctgtacaataaatctatcatctggtagcctgagcatgtctttagtccttgttgccagaaaaggtctctcatgcataatgcagtttaaaagtcctacagatgttcaaaggagtcacaggttagaggaagataaggctgagacacttcatggagaattgacacatgagctgccactgaaataagaatataccttaatcagtgttttagagatcatatcttcagatcgttttaatttataaagtataagaactacatgttttcacagcatgccaggagcaaaagatgaaaaaaagaactataagtgcttgacttgagtgttgattatatttttgtggacttattagacattttattttatataaatggtgtttcaaaaattaattcatttttatatattgtcgagttatttgttataagtagaaaacactatctgtagttttgactagaaataatacaaaagtgcatttctgaacctgtggttcaataattgccaatgatttataaaagtgtttcaggtgtcattcagtgtggtaatgcagttccatttcacttttttaaaattccataaggtaaaagaaaaagagcaataaaaggaaccagaagaaagtaaaaagggaaaaaagaagagaaagaataatcaaaatttgcaatttagagcctctgcccttttgactttcagtttgagttttattctggaaatgacttctgtgtcctctgtcatgtctctggcagcagagaggggaaggagggaagaaacctgcctttttatcctagttcactcattcaatgaatatttatggagaccttactaggtacctgtaactgtgctttttatcatatgccctctgcctggattttttttttctttttccttttatccgtggcaacttcctatttatccagtgagctgtagcttaaatattgcctactttttgaaatgctcacttattctacaaagttatactctccttctcctacatctcatagcattttacctatcctaccttcggaatatctgtttgtctgtccgactcttccatcagactgaccacttcaagtgtagaagacttttactctttttctcatcaccccttaacactaagcattatacctaacacataggagttgctcagcaaatgtctgatatggatgatgggtagatgggtgaccaactgttgacaacttgttgaaaagtataaaagatggaattttgcatctggctatcatggagtaggagactaacactcccattaagaacaactagaaaatctagattaaattccataagaatcgtatttgctggcatcagagagctgctaaggcagtttgctgaggtgagcccaacagtctgtgtattacttttctccttaggagagctggtaaaatcttaaatagctaaaagcagagcagagtttttggcaatctcatagtgttgaggaaagaaaaaacatgagtcttagctcaccaaagaggaggtggctctagttaatatccaggctttcaactggcaatccctacggatcaaaagtctacaaagtctagaacacctcttcacatagactcagtccctgctgatcagaatcctttctaaaggaagataacatcatttagagttactacaatttttcatacacagtgtttggcatttgatagaaaactaccagacagaacaaaaaaaaaaaacaggcctaagggggtgtgagggtgcagggtaggagagtagagaacagactaataactgacctagaaattagagttttcagaaatggacttcaaaataactacgataagttaaagaatataaatactaagattgagaattttaaaatataatcaaattgaaattttaaaaatataatcaagttgaaattctataactgaaaaatcactgacattataaactcaatagatggggtttaacagcatgaaaaactgaagtaggctgggcacagtgactcacgcctgtaatcccagcactttgggaggccaaggtaggaggattgcttgagaccaagagttcaagaccagcctgggcaatatagtgacacccctgtctctacaaaaaaaaaaatcaaacaaattagccatcctgtctactcaggaggctgaggtgggaggatcacctgagcccaggagttcaaggctgcaatgagctatgaccatgccattgtaccccagcctgggtgacagagagagatcttgtcttaaaaaaaaaaaaattggagttacccttaccccagttaagttacaacttaagagtgcattaataaaccaggaagaatgagattatattatggagagtattaacagtattgaaaagagcaaaaggatatacaggacatggtaaaaagtctaacatagatgtaattttggtcccagaaagggaagagagagagaatggagcaaaagtagcatctgaaatgataatggccaagaacttttcaaaagtgaccaatgaaaataagccacagattcagaaagtgcattgaattcttagcaggatttaaaaacactaaaacaacagcatgccactgtaaaactgctaaaaaccgaagacaaagaaaatcttgaaagcagcaagaagggggaaaagcacattactgtccaaaaagcaacagtgagactgacagttaacttctcagtagaaatgatggaagcccaagcaacaaaactatagtgattcattgcttaattaacaacagggatacattttgagaaatgcaatttttcattaggcaattttgtcattgtgcaaacatcgtagagtgtacttacacaaacctagatggtatgcctactacacacctaggccacgtgctggagcctattgctctgcagtaacttgcggaacctgtatggcatgttgctatactgaatactgtaggcaattgtagcacaacataagtatttgtgtttctaaacatagcaaaagcacagtaaaaataatggtattataacctcatgggaaccctgccatatatagtctatcattgaccaagatgtcattatatggtgcatgactatgtacatcttaaatgaaagataagctgccattctacaattctgtatttgcaaagatagccatcaaacatgaagacagtataaagaccttttttctttttctttttttttttttgagacggagtctcactctgttgtcaggctggagtgcagtggtgcaatcttggctcactgcaacctccgcctcctgggttcaagtgattctcctgcctcaggctccccaatagctgggattacaggcacccgccaccacacccagctaattttttgtattttttagtagagacaggttttcaccatgttggccaggctggtctcaaattcctgacctcaggtgatccacccacctcggtgctgggattacaggcgtgagccactgtgcctggtggagacatttatagagtatcaagaactgagggagtttccagtagacctgcacttaaagaaacaccaaagagatttctttaggcaaatggaaaattatcccagtcagtaatacagaaatgcgagaaggaatgaagagcaacagagagggtaaatatgtatttaaatctaaaagaacaccggccaggcaaaacagtaataaaacaataagataaaggaaaggaagccaatgaaattaaaatattgtatgtttctaggactgcctaggaagtggtaaaagtactgatttatttgaggtattaataaatcaaggtcacatattataattttcagggtagctactacagaaaaaaaattaaagaatatataattaacaagctaaaagaaaaaggatcaaatataaaatgtttaatacaaaaaagacaataaatgagaaaaaaggaacataaaataagtaggacaaatgaaaaacaaatattaagatggtagatataaactccaattcatcgtaactacattaagtattactggaaatacttcaattaaaagacaaaggtgatcaagctggatttaaaaaaaaactacttgctgcttgcaagaattataactacttagaaaaagtgtttggcagtatctgtgaaagctgggcacatgcatactcgatggccccagcaatctacttctacaaaggtactcacaagaagtatgcatagaatgtttaaagcagcattacaatggccacaaactagaaactacccaaatattcatcaacagaagaatgaataagttcttagataagtcttaaataaaatgatttttaaagtaacatttatcaaaagaagccagacacaaaagagtacacaatgtatgattccataactataaagttcaaaaacaggcaaaactaatcgatggtgttagaagttaggttagagatgacctttggaagggatggtgagcaaaaggggttaatgttctgttcctcattcctctgataattatatgggtatgtttactttataaaaaattcaccaaaaaataaaagaaaaaagaaaaaaattcaccaacctgtatatttatgacttgtgcattgcatgtatgttttatttgaataaaaagtttagtttttaaaaagtgtatcaggaaaggtattagaattggcagcaaggtcaattcagttatattttagtatgtaagaaacttggccgggcgcagtggctcatgcctgtaatcccagcactttgggaggccaaggtggcccaatcaccagaggtcaggagttcaagaccagcctaaccaacatggtgaaaccctgtctctactaaaaatacaaaaattagctgggtgtggtggcgggagcctgtaatcccagctattggggaggctgaggcaggagaatcacttgaacccaggaggcagaggttgcattgagctgagatcgcgccactgccctccagcctgggcgacagagtgagactctgtctcaaaaaaaaaaaaaaaaaaagaaaaagacaacatgcttcaagaaggaaaggttgaacccacagggaaagggagtgtttgaagatgctgaagagatgataaattaagctacttaaggacaaataagaggattcattcattcagctgaatattttaagtccctattataatccaagagctatctgaggtgttaggaatccaaaaattaataaggcttagtccctatttttagagtggtcactgtccaggagcaaaagatgccaatgatgatgtctgtaaaaactacatatttttaatactttccttgtacgagacactgtgctgtgcaggtttgcagtatcttacctgatatttacaacaatccaatgaggtagatgttatctcccttttcctgatgaaacacaggaaataatataagttttccaaggtctcaccaccaagataaaaacataagtctgtctgacgttaaagccgtgttcctgctggtactttacactgccattggatggtgtgtggttacatgcaacagtcacaatcaattctagtggggatgtcttggagaaggtggtatagatggcctgagcctcaaaggatgagcagacagagaataatgtttgatggcctggaagtggatttgaaggaagaagagtcttttctctgaataaaggccagattttcaaagaaggtggcagaaatgtataatgaacctgagacgactgagtgctgtttctcagggaaaggatctacctgctccatatacagactttcaaccaagtgtcttgttttcagaccttcctcctacaaagctgtctggcgtctccaattcctgactctgttctggacttctttgctttgtaaatgttggtatcatgtaattttatagccagattgttttaatgtttcataatgaacattttcccaaagcaaactgcttgttgcaaaacaaccgtatctttaagccagcattgtgggagagttctagattcctgccagagttaaacccaaggtagtcatatgtgtaattcaagtgctcataaaacagctggaaggaaaaaaaatttggatataaaataaattataggtggggcacagtggctctcacttgtaatcccagcacttcggaagcccaaggtgggagaattgctgagcccaggagttccagagcagcctgggcaacatagcaagaccttgtctctacaaaaaaattaaaaaattagccgggtgtagtgttgtgtacctgtaatcccagctactcaggaggcggaggcacaagaatcacttgagcctgggaggcggaggttgcagtgagccaaggtcatgccactgcactctagcctgggtgacagagtaagactctgtctcaaaaaaaaaaaaaaaaaaaagtctgtacggatacagggagcacactgcttccttttaaaggcaagaccgctatgttgaacattccacttctgctaaaccctaattatccagaagttataagactgcgaagcaatctagcttggtggccatgtgctctgtgctatataagaagctgaggtcagtactggggacaaccaatgctctctgccatattctgagcttagtaaacttgagagtgttgaattaatttagattaatgtagctgattggtaaattctgatctttgtggtagggcttgcttctacttgtttatattaaaaaggtaatgcagaacttaggcctggcgtggtggctcacacctgttattccagcagtttaagagaccaaggcgggtggatcgcttgtgcttaggagtttgagaccagcctgggcaacgtggcgaaaccttgtctctacaaaaaatacaaaagttagccgggcatggtggcacatacctgcagtcccagctacacggaacgctgaggtgggaggatcccttgagcccaggaggcagaggctgcagtgagctgagatcgcgccactgcactccagccaggacgacagtacaagatcttgtctcaaaaaaaaaaaaaaaaaaaaaaaaaaagcagaacttaaaacataaaattgctacatatttatatagatgaaatcactaagatttttacctatatacaaacctaacagaatatggacgcttactgctttgtacttctttattcactggaattaagtaaaattctctttccattttggccagtgaaatcaactcttgcagggttacttttgcttttaatactaaccaattaaattctagagcctaggcttaaaaatgcaaatctttataataaaaagagactaagatagaggtgaaggagatgtccctttagagcagtagaggaagagttatggttctatttcattgaactacctcaaataatctactttgatgaggtgttaaattaagaacattcataagaacatcaatcttgttttcctgtgcaatatgaaaacttgtgtgaatatttataggctagcaacttgtttttcttttctctgttcttggaggtataatttgaacaacagggccttgtaaaagtcttaagacttttgaaattgttaaatactgcaaagataaccttcaaagagaagtgtgtaatcctgattggctgtcttctagatagtttgttgccttggtgataacactaatttacatgtctgaattcctaaacaacctaaaaacttgtatctttgctctgtaaagtcagaagagaatcattggcacatttctcttttcccgtatgaataatggagagaaacaaattcctgttacactggttgatttcttattttaataaagatttaacaaaggaaattgcctttgtgtgtgtgtgaaaatttagtatctaatatccttatctttgggaattcatgtaattctttttctcaagttatttaacagtgttcttcttttttctacatagactgtccttcatctacttggattcagttccaagacagttgttacatttttctccaagaagccatcaaagtagaaagcatagaggatgtcagaaatcagtgtactgaccatggtaaaaggcttattcttttctgtttgttttttcacaaattagtgaacagtgttcattaatttctttaaaaattgattttgtcacatctgacttttaactgggtcctgagatttaatgtacaacttttatttgacaacacacaagctacaggcagtggaattgggtatatatttaatcctggagttggaagacagttgttggacaatattgtccaaagcagggtctatttaacatgcatatcctgtgccaggcactgtgcttaagcacctcatatactttacctggctttatcataacaaacctatcaagtagtgtattattatcattttattgagagatgaaattaacttcagttatttgttcaagcttacacaggaaataattgacaaagccaggattttaacccagatctgttggattttaggcttagagttcttaatcacttagttattctgcctggaaagtgctgaagtcagattacataggaccatgaagtaagactgtagtaaagaagagtacattaaggaaaaattataaaaaggctatttgctagaccatacactcagtgttctctaggaaatagatttataacatcacattgtaaaatatttgaagtgtctttttttttttttttgagacagggtcttgctgtgtcccccaggcttgagtgcagtggcatgatcatagctcactacagccttgaactcctgggttcaagtgatcctcccatgtcagccccctgaatagacaggactacaggcgtgcgctagctattttatctgtttgtttgtagagacagggtttcactatgttgcccaggctggtctgaaactcttggcctcaagtgatcctctcaccttggccttccaaggtgctgggattacaggtgtgagccactgcatccagccaaaaaaatgtctttttttttttttttttttttttttgtgagatggagtctcgctctgtcgcctaggctggagtgcagtggcgtgatctcggctcactgcaagctgcgcctcccgggttcacgccattctcctgcctcagcctcccgagtagctgggattaccagcgcccaccaataggcccggctaattttttgtgtttttagtagagacggggtttcactgttggccaggatggtctcgtctcctgaccttgtgatcaggatcaagtgattctcctgcctcagcctcccgagtagctgagattacaggggtgcatcaccacacctggctaattttttgtatttttagtagaaacaaggtttcaccacgttaaccaggctggtgccgaactcctgaggtcaggcaatccacctgcctcagcctcccaaagtgctgggattacaggtgtgagccaccatgcccagcctaaaggtgctttttattactgttatattcttcaacaataacatttggaagaatgtatcaaattgtactagggataccttaatcaaaatcatatacatttattatagaaggaataaatgtaaacaccaaagttattaatcttattaaagcgccacatagtacttaatacatatatgttgtttccctttgaaaaagtcttacatgaacattggctctgtgaaaacaagacgaaatttccaacatgccaatatggttttatttcaaggcagttgaaataaaataatagcaattttgctggtcttagaatatttttaaaacaagttttcccctgcaactattttctttcctttcttcctttttcttcttcctcttttctttctttctctctcttttcatttgttctttctttctttcccttttttttttttaatggagtctcagtctgttgcccaggctggagtgcagtggtgtgatctcagctcattgcaacctctacctcctgggttcaagcaattctcctgcctcagcctcctgagtagctgggattacaggtgggtgccaccagacccaactaattttattttattttatttttttttggtagggacaggatttcgtcatgttggccaggctggtctcaaactcctgacctcaggtgatccacccgcctcagcctcccaaagtgctgggattacaggcatgagccaccacgcccagcccctacaactattttccatcccttaaaaaaaattatgtaaagtatactgagacataaacataatttcaatgacataaatcatattagaggctgagagccaagacatgtccctgtaaccttattagcttgtatgaactgtaagatcaaagtttattgtccagctctaatatatatagaaagttaagcaactatttattatttttatgtacctagtgattttcactgattctggaatatgctgactttttttctctttaaaataatttcaggagcggacatgataagcatacataatgaagaagaaaatgcttttatactggatactttgaaaaagcaatggaaaggcccagatgatatcctactaggcatgttttatgacacagatggtaagtgatatttacctcgtgggacgtgactttgttgttctttttttaatgtagcaaaactgatggcttttcaattctctggttcaagttgatgattcctgtggcctccaggtgttaaattttcagtgtgcacctatgatcataccacagcttgtatctgcatagctcaatgagttacacagatgaaagccacagttgattcttgtctggattaaaatgtggctctaaggctcagtatttttgtaatatagcatgatcataaaggtaagtattagtgtctgcaactaataggctaaggatagactaaattatcacggttctattatataaacatatttatcattactgtagacaaagtacatatgtacaaatataaaagtacatatgtaaacattttcaaatgaaatgaaattactaaggttactattaatatttaactatttttcttgacttttttcccatgagaatattatacatgtatatatttttgcgaaaattggatcatcttcagaaacctcttaaatcgatagcttttgatgatttggctgtacacaggatatattcctccttctaattttaatcctacaatcagaagtttagaccagacataccttagtaactttccacctttaatcctacaccctttttggcttgaactccttttaaaactgtcataatgttgaaaatattccttaattgacagatgcgagtttcaagtggtttgataattcaaatatgacatttgataagtggacagaccaagatgatgatgaggatttagttgacacctgtgcttttctgcacatcaagacaggtgaatggaaaaaaggaaattgtgaagtttcttctgtggaaggaacactatgcaaaacagctagtaagtatgaccgaaggtgtttttccattctagaaggggcagagaaagtaggactggttttaaaataattttgagataagtgtgcctcaataaatagaataaggctaggcacagtggctcacgcctgcaatcccagcattatggtaaactgaggcactggattgcttgagcgcaggagttgaagaccagcttgagcaacatggcgaaaactcatctctacaaaaaatacaaaaatcagctgggcatggtggcgtgcacctgcggtccgaaatacatgggagcctgaggtgggaggatcaattgagcctgggaggttgaggctgcagtaagcctgggcaacagagcaacactctgtctcaaaaagaaaaaaaaaaatagagggaataagacttcagaatatattaataggatggtttgctcttaagtctcttggaactcaaattgtaattctcttacatttaggaggtaaggccagatcacatgaaataggcatacttttattctgtctgcttagtgtttccaagaggtcaaaaatttcttgttcagaggaattattataatctctttgcatgtgatttgagtgagcagaagtggtgaagagtaatcatgtataattccagaattctcgtgttactttacaaggaattagattctccataagaacatcctttgtttaggcaaacaaaaactacatcatagtttataatttatttggaattaaaatgttttcttctgacccgtcagaaggttttttttttttttttttttttttttgcgatgcagtctctctctgtcgcccaggctggagtgcggtggcgcgatcttggctcactgcaacctctgcctcctggcttcaagcgattctcctgcctcagcctcccaagtagctgggactacaggcacccgccaccatgcccggctaatttttgtattttttagtagagatggggtttcaccatattggccaggctggtctcgaactcctgaccttgtgatctgcccacctcagcctcccaaagtgttaggattacagacgtgagccactgtgcccggcccagaaggttttttaatgaacagaagttatcctggcccacaacagtattttgatgttatttgttaaaacactacacagtttgttattctaagttttagaaagtaactgttttctcattaaagaaaattaaattttcaaaatgtgggctgggtacagtggctcatgcttgtaatcccagcactttttgaggctgaggcagcctgggtcaacatagcaagaccaccatctctacaaaaaaagtttttagtttttttaatgtttcaaaatatgaatttagggtgcttaaaacatgatataaacaaccatttccccattttgcatcctcagtattttccccttctacttcaactgtaaaccaaaactccacatcctgctttatttcctttctttacatatggaagtatgtacatttctttacttgctacagcaggatccttgactactgttagttttacagtgattatcaaaatgttcgtcatagttttatggtagcagtattcaacttcaaatatttgttttttaaaaaattggttcttttcaggttctgcacctcagggtgccattacataaacaaagtttttaattaaactataacttcaaaacatcagaaatagtgagtacatccatactaaggtagttagggtcacagttgtttaattttatgacatttgttttctagactctccaaccagaatcctcttttttcctttacctcattattgtcttttcctcaagatcccgtattcttttccaccaaccaagctaatgcatgttgttagccctctacatctttctttaaaaactaagttcaaaatagacaacagagacaactaatgaggcaaagaaatatggattttaggctatgaatatattgatattaagctactaataaaagggtttatttttaatgattgtgaacatgtttaaagtcaatcctataataaagcataattaaattctaatttttattttcttttgtattttagtcccatacaaaaggaaatatttatcaggtaagtaatgatttggtctttaaatttttcccacaattaaaaatatatcaaaattctggcatattaaaatacattacagatccctttttaaaattatgtaatataaagtagaacaatttttataagaatgtagaaaggtctcttagtaataagactcaacattttaaatatattctacaatatgtagttttgtctggcttcttttactcgggaatatttttttagattcatctgtgttgtgcacattcactgaattttttccctaagtgtcataatactttataaatatatcatatattgtccattctcttgtttatggatatttggatcacttctagttttaaactaatttgaatagaactgcctatgaacattctcccacatcttcttttggacctatgttctcattttctttgagtatattctagaaagtagaaagctgggtcatagggcaagtatatatatttagatttattagaaaccaccaaataatttttcaaaatgcttgaatcatgttactactggcagtgcgtccaagttccatttgctccatgttcatcccatgatggtattgtcaatttatttttagccattcagatcaggtgtgtagtgatcttcttgtggctttaattttcatttacctgataatgatgttgagcacctttcagatgattatttatttgtatatcttttgtgaaaaggcttttacccatttgcggagtgcagtggtgtgagcttgactcaccgcagtcttgacctcctgggctcaagcgatacttccacctcagcctcccaagtagctgggactacaggtgtgggccgccatgcccagctaactttttttgtacagatggagtttcaccatgttacccaggctagtctcaaactcctgggttcaaaggatccacccgcctcagcctcccaaagtgctgagattacaggcgttgggccgccatgcccagccacccatattttttttgagttgcctatatttttggtaggaatttttaaaacaaattttgtttctttaaatttggatatgggccgggctaaataatacaaaacttagccgggcacggtggcgggcgcctgtaatcccggctacttgggaggttgaggcagggagaattgcatgaatttgggaggcggaggttgcagtgagccgagatcacgccactgccctccagcctcggcgacagagtgactccatctcacaaaaaaaaaaaaaaaaaaaaaaaaattggatgtgagtcctttgccagttttctatttgggggttactacgatagaactatgacagaggaggagaaaagaagtgttgggtttgtgtgtatacaggaattacatcctcattaatctataggaagaagacaataaataacaagcattagtgtggatatgcagcaattggaactcttgtggtaggaatgtaaaatggtgcagcctctgtgaaaaacagtgtggtggttcttcaaaaaaaattattaaaagaattaccgtatgatccagcaattctgagtatgtatcccaaataactgaaagcaaggactctttaatatccgtacacccatctgtttgatatttgtgtatctatgttcatagcatcatttgccatagccaaaaggtggaagcaacccaagtgtccactgatgaagggataaacaaaatgtaatctgcacatacaactgagtatcatttagccttaaaagagaagaaaattctgacacgtgctacaacatgcataaacctgaaggacattaggctaaacaaaataagccaggcacaggccgggcgcagtggctcacgcctgtaatcccagcactttgggaggctgagaccggtggatcacctgaggtcaggagtttgagaccagcctggccaacatggcgaaatcccgactctactttaaaaaaaaaaaaaaaaaagttgaaacccggaggcagaggctgcagtgagccgggatcactccactgcactccagcctgggcgagacagagcaagcctccatctaaaacaaaacaaaacaaaaccaccacaaaaaaaaaagaaaaataagccagtcataaaaggccaaattctgtataattcttacctatagtatgaggtacctaaagtagtcaacctcaccaagaaactctttgccaagggttaggtcaggaagaaatgggagtttaataggtacagtttccattttacaaaatgagttgttctgtgaatggatggtggtgatgtagcacaacgaagtgaatgtacttaatgctactgcacacctaagatggtatttgttacatgtatttctttaaattacatttttaaaaaatcaagcagcggaagcatgttatttagacatggagacagataaatagctgaagcaacagtagtagttaactttaaggaatggatgttcaggtgagtagtggagcaggagcacagtattttcactacgtatcttctagaactatgacttttaagctacgtaaatgtattactttggaaaaaataaagatacacataagttggaacatttagaaaaaaataggtctgtggcttcaaaaacgaatagttgaaagatctggcaacatatggtcttcagactacaagaaaagtaatctctgaattcattacatgtatgcatgtatatatgtgcatgtaaatctgtattaaatatgtgtatgtaatttagtgagtaaacactatgtgtcaggctttctcctaaagttagacattgaagacagagcaatgaacaaaacaaaagcctctgctctcaagcatacattctagtgggaagggtaaaacaaaactatgtgagatggtgacacatcctatagggaaaacaggcagataaggatgaattccagtgcaagaagggaggtgttgcactttatttttatttttagtacagtggccagggaaggcctcatttaataaggccatgattttgcataatgaagacctaaagtcaaagtgaaagcctgtcatgtagatagggattaggcttagtctgactttaaggagtaaaacagtagagaacatggagttgaagcagcaaggaaacaacccaacagttaagtgtagctgccatcttggcaaacagggagttccccttcataccaagggtaagctggaagactcccttgggaagaatactttcaacagaattaaggtgtccatagtcggatgcacctagtgtggcaagcacacaggaaaggtcaagttagtccaagctgggttcagcaaaggttactggagaggatgcaggagttaaatcttcaaggataagtagacatcaggcaggagacagaggacaggttatctgaggtgacaaatcaggaccaaggtgaagggttgaatttttgctgtcacttactaaatggacttgggcaagtcatttaaaccctgtgcctcagaattaaatgcacagttggcactctcacctgaggctacagcctaacaaaatgtttcatatagtttttgaggtggattatgtatagttcgggcctcagatgatcagtgttagaactagagcgttaaggtagcagtcagatagataaataggaaacagaagaaaaatgtgtagtcttcacaatttatagagaggaagtctataaatttccagattggttgactggacagtggctatttatttgcaggaattcaggttagaagaaatttggaagtagggctacacacacatatttatctatggatttacataaaagacttgaatggttgagatcaaccatggaaagctttgagtccacaaagatgactaggaagcagtcgtctgaaaaatagagcagtcagggaagccaagtgagtaggagataacttcaaggctggacggctaacaatctagttgccccaggatgaactaaaacatccacgtgtttaagcatgtagtcataaagagatcattggtgacctctgccacagaaatttctgaagaaatgttgagaaaatggattaaatgggcttaagaaatgaaaaagtgagcttgtttcagtgtttgctgtttcctcaagtaaggaacattgttaggctaaaaagtatttttcctaaattttgtttctttaaaaaaaacaggatttcatggtaaatagctattatatgctgtcaagcaagcaatggacctttcctttaattaatgaggtagataatacatttcctaatgtaaaactatcctaacctccagatataaactacacattgttttaatacactgcttatcagttgccttgacaattgactttgtcacacattttagtagtatgcagctagctaactaaaacccctggaggcaactttggctaaaggtcacttgcagaatgtgaaaagcagcatatatacttactgtgtctcgcccatcttggacactgtccagttcttaggatacccggtcttttaaagctattatgattgagtctgtaaatatttctcaattactatgatctttcagacttttctatattactatcatgtaattcctatttaaagtagatgctaccgattatgaagccattattgtacaatcattagtacactgaaataaagtgtgttcacactggtaaagttgaggttctacactgaaaatggactatgttaattctaataaaactaactacggggctgggcacggtggcccacacctgtaatcccagcactctgggaggccgaggggggcagatcacttgaagccaggggtttcggaccaacctgggcaacatggcaaaactgtctctactaaaaatacaaaaattagctggacatcatggcgtgcgcctgtcatctcagctacccaggctgacacatgagaatccctggcacccagaaggcggaggttgccgtagccaaaaactatggaagaaccccatttgaactcagtttacggaattcttggctttttttcattttaatgtctttaataataccaccaccaaatccaccctcacaccttaccaaatctttactaccaaattttatttcagaattaaaaagatagttcacttggggccatttcctcactcctcccaaaaaatcctgtcaatatttgaaaagtatcaatggctctccctcactttaacaaataatttggccaaatttctctcactaatcttaaattcttccaggtctcattttaacaagcaccagttgaaataatgggtattatgaatatatttaataataaaaaatctgtcaaaataattctgaaaagttttcacattctttagagtcgattactatacataatacaagtacctgcccaaaagtatgtttgttctcctaaaccaggttattttcctcaatttattaaactggattcttatagaaagattacaagggtttgaataggcaagaactgcaaagttgtgcatacttacagaaatttagggaaacacacaaagaatgaatgaatgcaaagacacacaaaaccaggtactttactgcaggtcttggtgtttttttcagttaaacaaatcttaaatccccaaagccaggtgaaccattaaacctcaataatgtaacacattatctaaatactagaattcctaaagggaacagtaggtctgaatttgtagtcttagatcagaaagtaaagcgaaggaagacaaactggagcatggtatatacttgcgtgatttccactatggggtcttcagtagagtggagaagtgcacaacttcctagaaaatttgagtactattcttcaggctaagaaatataaatcttgttagtaacaaatgcgaaatttttggttttctcatcagtgtgggaaattctcaacacagagaaatgtttacacaaaactacagttgtgctcatcaaaatatacttgcaacttgtgtaccctaatttcccaaatactcttttgtcttttccttttcagataaccacattttaatatcagcattggtgattgctagcacggtaattttgacagttttgggagcaatcatttggttcctgtacaaaaaacattctgattctcgtttcaccacagttttttcaaccgcaccccaatcaccttataatgaagactgtgttttggtagttggagaagaaaatgaatatcctgttcaatttgactaagtttttggtaatcttgcactaagacatcaacaaaatgccctggcagagataacttgggaaagattttaatataaaacttgacattggatattagagctttaatggtattccttattccagtaacatttttatgtactcatctgctgtgaaaagtctttaggttcattaaaaaaacaggttttagaaatgatcttagatctaatatagtgattttaagcatcccgtcaaaggcagaatctgtcacttgaatgaaggaaagcttaaagcccaagcagataaaaataaaagcccagcctatttgtcttgcctgctgtatcttccctatttagttgacccactttagtttatatgtttattagtaaacatgaaatggggaataagtgattttaagtacatcccatacatttaaatatctttgataattgttatttttttggcagataattcctctagaatgtgtatctttttatgatttagatgaagaaaattttacaacttttaacaccccacaccaattttagtttcattacttttacacacaccattttatcacaaatgactcaagttttaatgaatgtttataaattatttgaaacaaaatatgatcgctgtgtccaggatggcatagagaaagctggcaattaggttaacacttacatattatagtgcccctttaaggatttctctcttgccaccataccttttgtactttcccctatacaagatgtatctcattctcctcaagcatttataaatttttccttcaatgacatgaaaactgtgcaagcaaaaaccgaagaaaaacacttaagtacaactgtagtgacagtgatcaaagttttcagtgcatttattgtacattttaagaaaaaggtgaaaatcatttggggagtaaaaaaatgaaaaagctgaaacgagtaattttcctcaccatcaataaaccaaaaacaggaaagataaagaatgtataaatttcacgtaaattagtcacgtatcacttatcaatggggatacgttctaagaaatgcatagttagggaatcttgtgtgaaaatcagcttgtatttacacaaacccagatggtagagcctattttgtcccaaacctacacagcatgttactgtgctgaatactgcagacaattgtaacacaatatttgtgtatctaaatatagaaaaggtacagtaaaaatatggtctactaaggaaacactgttctatatgtggtccattactgactgaagtatactgtctagaagtctgaggctcaaagaaaagtaatccctcttctgaatccacaccccatcaattatcttactttcttctggggagatagatagatatactatctcactagcttgactaatggcaacaaagttccagcttgtgtagtctctttttattgaccacatgaatcgaaaacactcatcacaattaatggcactatcattaatgagacatgagtaactaaaaagtgatagaaaactattaacagtgcggctacatggtactgaaaatgcaggcattacaccagctgttacacaagcacaagcatgctctgtaagagctttacatttctgagattttgtatagtgattgagatgtctattttattattgatagactattactaatgtcaatattgaacactaccctggaattcctgcctggttttcctacccaaattgtaccactccttgaagaactacaggcacagtaaaaaaaatatggcgtattatgtgaactaaaagagttctaaaggagttcttaaaggagtggtagaatttgggtaggaaagtgattaagtccaacttaaaaccaacagtctcaaacgtctacaactacaatgtccaatgagccactagccacatgaggctatttaagtaaatttagtttaaaatccagttttcgaattacattagccacattgtcaagtgttcaaatcacaggtggttagtggctactgtactgggcaacatacattatagaacattttcattataggaagttttattgggcagtgctgctcttaaatcctaccttccactcaactcccatacaactttcttttgtacattttgatactttctacctaatggcagctcttccaaaatagctgctttaaactctgatttaattttcaatatttggtttcatttttcaacaggccaagaggcctctggtaatgaagtgctatatatatatatatgatggagtctcactgtgctgcccaggctacagtgcagtggctcgatcttggctctctccaatctccgccttgcaggttttcaagcaattctcctgcctcagcctccttagtagctgggaccacagacatctgtcaccacacccagctaactttttgtatttttggtagagacggggtttcgccatattgactgggctggtctcaaactcctgacctcaagtgatccacccaccttggtctcccaaagtgctgggattacatgcgtgagccaccacacttggcctacattttttctttatataccagaacatctataacaggcaccttatctactcattagtgaagagataattggattacacaggcaggcttgtttactacatccagaatgtagaaactgctttcttcaacatcttggttctagctagtaataacaatataattctttggcagatattcagaataacattttaaactacattttcttagaaaattgcattcttgtagtgagcagtgtatggtctcttttgttcagaatttaaaactgataaccaatgaaagccttttctcttattcctctaccgtcatttacatgataatctgaagctaatatgacaatatttaaatactaagtggtactagggaactacaagaatactgtaaagcttaagccattgttatcactgtcatttagcatttaataacaaaactatacagaattatgtgcataccaatgaatgttttgtaccatctagttaaattttttaaataaagttttatgggttaagcagaagacaactgtcatactgaattttattaaaagtatatatacttcaaattcaaagcatcccttaggacccacagaatatattaaaactaccacccttaaattttatatttttgctttaagacagacaatgcaaaggtaactggcaagaggtgagcaaatgttttagaacatttatattattgcttaaaatgagatttgaaattgtaataaaattcttggttatgaagtctgatgtcttctttgagcaccagtttaaaaggaaacatttcaaacagtaaaataaatcagtgtgtactttattttgagccaatgttttacataagaccaacaaactaagtgtgggcaaactgttcaatcacttttgagaaattacatggttcctatttaggtttgaaaaagtagtttgttgccataacatcagaccttaaggtgttttaaagtgtatcctaagggtactgacagaagaaaaatacagtgttatggaagtatacacaactcatgaccacacagtatagatccattcatggtgaataatgttcatctgttctccaaaggtagtgaaaaatattaagtcatcaaataaatgctcagaattaccaaggaacagttaaaaaggaccaggatccaaacaggttatttatatatttatctataaacaagtgacaacactaaaaaacacattgaaaaccagtgttttaaactctgaatgtgggatttaaaaatattccattaacttgagattgcaataatttttttaaaccaaatagcatttgactggcaagtctcatataatttacagctgtaatattcctaaaaggaatatctacatactatagcctatcaaatataatctgcattttgcaaatcaatacaaaaatctatccatcatttatacattttatatttatatactttttaaaaattagattttattgctaggtgaatttggaattcaaattaaatttggaaacttaataatgctcttctcataaccttctccccattctttttttaatttttaaacactggaagcaaatgattatcaaaatgagactgcaacttaaggcattttaaaagaaaaaataattaggtgccatatagcattttatttcaaaagtatattttgtcccacttttctcactagcaagagtaaaacacaaaccttttttcataaatatgactacagtaatcataacacaaaaaagggttgataggcattgcttagatatttaaaacaagggtaatactttcccactcacctaaaagaaaaaacctttttgattaccagtttataaacatcggatttgctatgttaaaaagtccagcagaattttaattcagcaacactggagaatgaatatatatatacaggcatacatgtgtatgtatatattcattttatatatagtcaatgtatttattaggcattcccccacaaaagtaatttatatttaattgccatttttaataaacacttatgttcacagatcatccatctgtcttatatgaagttaggcaatatcaaatgtcctgttatggtctccgtcttcttcggaatcatcctctgaagctgttgaaagaaaacaggatgatcaagaatgattttaagggggataagaatgggaagtgtaaatatgaagccagaacagcaatgacttgtaaaaacaaaaatcccaatttagtatattgtttgatcaaaatgataagttgtatgacagggttagggtggtctgcctgtgggtgatacccaaccacaattctccccgcccccacaccgttttgtaatgttatatcaatttttataaaaatgaaagtgcttaaaaatgtggttcaagagcatcttctaaaatgtttctgagatcaatctgtgttcatagtagctaatgatatataaacactaggaaaaaagtcaacttaaggcaagtgctctcacacacctgagtaaaatctggcttattaccgctgcatgtttggtgcatccgatgagaatgaaaagaagtgtacatagaataagaggttatgggtgttcttgcaatttacatcaaagctatgtctatacaatgccaagataaaagttc

SEQ ID NO: 10 - DNA, 3740 bp, Homo sapiens
gagaggccggcgcgtcggcgacggcgggtgggcgtgggcggcagtacgaggcccggcgcgacgggcgcgaggacgacggcaacgacccggagcggcgacgacggcagcgcctgacaggaagtagatgaacctaagtcaaggttctgtcaacaatgtaaaaagaggttcttcggtagtttcatctttcgtatctcctacagtctttagtcacatgactggtacctcgcctgtactattcgtatgtattacttcttcttttacgaaaatatgacctatgaaactttttcgttacctttccgggtctactataggatgatccgtacaaaatactgtgtctactacgctcaaagttcaccaaactattaagtttatactgtaaactattcacctgtctggttctactactactcctaaatcaactgtggacacgaaaagacgtgtagttctgtccacttaccttttttcctttaacacttcaaagaagacaccttccttgtgatacgttttgtcgatagggtatgttttcctttataaatagtctattggtgtaaaattatagtcgtaaccactaacgatcgtgccattaaaactgtcaaaaccctcgttagtaaaccaaggacatgttttttgtaagactaagagcaaagtggtgtcaaaaaagttggcgtggggttagtggaatattacttctgacacaaaaccatcaacctcttcttttacttataggacaagttaaactgattcaaaaaccattagaacgtgattctgtagttgtttacgggaccgtctctattgaaccctttctaaaattatattttgaactgtaacctataatctcgaaattaccataaggaataaggtcattgtaaaaatacatgagtagacgacacttttcagaaatccaagtaatttttttgtccaaaatctttactagaatctagattatatcactaaaattcgtagggcagtttccgtcttagacagtgaacttacttcctttcgaatttcgggttcgtctatttttattttcgggtcggataaacagaacggacgacatagaagggataaatcaactgggtgaaatcaaatatacaaataatcatttgtactttaccccttattcactaaaattcatgtagggtatgtaaatttatagaaactattaacaataaaaaaaccgtctattaaggagatcttacacatagaaaaatactaaatctacttcttttaaaatgttgaaaattgtggggtgtggttaaaatcaaagtaatgaaaatgtgtgtggtaaaatagtgtttactgagttcaaaattacttacaaatatttaataaactttgttttatactagcgacacaggtcctaccgtatctctttcgaccgttaatccaattgtgaatgtataatatcacggggaaattcctaaagagagaacggtggtatggaaaacatgaaaggggatatgttctacatagagtaagaggagttcgtaaatatttaaaaaggaagttactgtacttttgacacgttcgtttttggcttctttttgtgaattcatgttgacatcactgtcactagtttcaaaagtcacgtaaataacatgtaaaattctttttccacttttagtaaacccctcatttttttactttttcgactttgctcattaaaaggagtggtagttatttggtttttgtcctttctatttcttacatatttaaagtgcatttaatcagtgcatagtgaatagttacccctatgcaagattctttacgtatcaatcccttagaacacacttttagtcgaacataaatgtgtttgggtctaccatctcggataaaacagggtttggatgtgtcgtacaatgacacgacttatgacgtctgttaacattgtgttataaacacatagatttatatcttttccatgtcatttttataccagatgattcctttgtgacaagatatacaccaggtaatgactgacttcatatgacagatcttcagactccgagtttcttttcattagggagaagacttaggtgtggggtagttaatagaatgaaagaagacccctctatctatctatatgatagagtgatcgaactgattaccgttgtttcaaggtcgaacacatcagagaaaaataactggtgtacttagcttttgtgagtagtgttaattaccgtgatagtaattactctgtactcattgatttttcactatcttttgataatgtcacgccgatgtaccatgacttttacgtccgtaatgtggtcgacaatgtgttcgtgttcgtacgagacattctcgaaatgtaaagactctaaaacatatcactaactctacagataaaataataactatctgataatgattacagttataacttgtgatgggaccttaaggacggaccaaaaggatgggtttaacatggtgaggaacttcttgatgtccgtgtcatttttttataccgcataatacacttgattttctcaagatttcctcaagaatttcctcaccatcttaaacccatcctttcactaattcaggttgaattttggttgtcagagtttgcagatgttgatgttacaggttactcggtgatcggtgtactccgataaattcatttaaatcaaattttaggtcaaaagcttaatgtaatcggtgtaacagttcacaagtttagtgtccaccaatcaccgatgacatgacccgttgtatgtaatatcttgtaaaagtaatatccttcaaaataacccgtcacgacgagaatttaggatggaaggtgagttgagggtatgttgaaagaaaacatgtaaaactatgaaagatggattaccgtcgagaaggttttatcgacgaaatttgagactaaattaaaagttataaaccaaagtaaaaagttgtccggttctccggagaccattacttcacgatatatatatatatatactgcctcagagtgacacgacgggtccgatgtcacgtcaccgagctagaaccgagagaggttagaggcggaacgtccaaaagttcgttaagaggacggagtcggaggaatcatcgaccctggtgtctgtagacagtggtgtgggtcgattgaaaaacataaaaaccatctctgccccaaagcggtataactgacccgaccagagtttgaggactggagttcactaggtgggtggaaccagagggtttcacgaccctaatgtacgcactcggtggtgtgaaccggatgtaaaaaagaaatatatggtcttgtagatattgtccgtggaatagatgagtaatcacttctctattaacctaatgtgtccgtccgaacaaatgatgtaggtcttacatctttgacgaaagaagttgtagaaccaagatcgatcattattgttatattaagaaaccgtctataagtcttattgtaaaatttgatgtaaaagaatcttttaacgtaagaacatcactcgtcacataccagagaaaacaagtcttaaattttgactattggttactttcggaaaagagaataaggagatggcagtaaatgtactattagacttcgattatactgttataaatttatgattcaccatgatcccttgatgttcttatgacatttcgaattcggtaacaatagtgacagtaaatcgtaaattattgttttgatatgtcttaatacacgtatggttacttacaaaacatggtagatcaatttaaaaaatttatttcaaaatacccaattcgtc

SEQ ID NO: 11 - DNA, 1122 bp, Mus musculus
aatgccacaagtgctatggattagtcaacagtgctccaccaatgctctgtcctggttcctatccttgcactgatgttatgtaagatgctaacatttggagaagctgccgcaaggattacgggaagttctatttatttttgcaacattttagaaagtctgagattacttcagttcaaatgagaagtttatctttaacgaagagaagttggagtctgcggtgtgtccgcgcttggggatctgagcgtcccagcagtgcgaccctgggctccactcccccgcctcgagtgggaggcgtcgcaactgagctgggagctgcgcacccgacaagcaccgcccccggcccgctctcggcgccgcgcagtcatgccccacgcagcgctgtcctcgctcgtgctgctgagcctcgccactgccatcgtcgccgactgtccttcatctacctgggtccagttccaaggcagctgttatgcttttcttcaagtaaccatcaatgtggaaaacatagaggatgtcagaaaacagtgcactgaccacggggcagacatggtaagcatacacaatgaagaggaaaacgcgtttatactggacactttgcaaaagcgatggaagggtccagatgatctcctgctaggcatgttctatgacactgatgatgcaactttcaagtggtatgatcattcaaatatgacattcgacaagtgggcagatcaagatggtgaggacctagttgatacctgtggttttctgtacaccaagacaggtgaatggagaaaaggggattgtgaaatctcttctgtggagggaacactttgcaaagcagcaatcccatatgacaagaagtatttatcagataaccacattttaatatcgactctggtgatcgctagcacagtaactctggcagttttgggagcgatcatttggttcctctatagaagaaacgcgcgctctggcttcacctctttttcacctgcaccactgtcaccttacagtgatggctgtgccctggtagttgcagaagaagatgaatatgctgttcagctggactaagagtttggtaatatcaggccagcatattgartccattgacaawaatttcctgtgcaaggttttcatataaaa

SEQ ID NO: 12 - protein, 228 aa, Mus musculus
MPHAALSSLVLLSLATAIVADCPSSTWVQFQGSCYAFLQVTINVENIEDVRKQCTDHGADMVSIHNEEENAFILDTLQKRWKGPDDLLLGMFYDTDDATFKWYDHSNMTFDKWADQDGEDLVDTCGFLYTKTGEWRKGDCEISSVEGTLCKAAIPYDKKYLSDNHILISTLVIASTVTLAVLGAIIWFLYRRNARSGFTSFSPAPLSPYSDGCALVVAEEDEYAVQLD

SEQ ID NO: 13 - DNA, 1122 bp, Mus musculus
ttacggtgttcacgatacctaatcagttgtcacgaggtggttacgagacaggaccaaggataggaacgtgactacaatacattctacgattgtaaacctcttcgacggcgttcctaatgcccttcaagataaataaaaacgttgtaaaatctttcagactctaatgaagtcaagtttactcttcaaatagaaattgcttctcttcaacctcagacgccacacaggcgcgaacccctagactcgcagggtcgtcacgctgggacccgaggtgagggggcggagctcaccctccgcagcgttgactcgaccctcgacgcgtgggctgttcgtggcgggggccgggcgagagccgcggcgcgtcagtacggggtgcgtcgcgacaggagcgagcacgacgactcggagcggtgacggtagcagcggctgacaggaagtagatggacccaggtcaaggttccgtcgacaatacgaaaagaagttcattggtagttacaccttttgtatctcctacagtcttttgtcacgtgactggtgccccgtctgtaccattcgtatgtgttacttctccttttgcgcaaatatgacctgtgaaacgttttcgctaccttcccaggtctactagaggacgatccgtacaagatactgtgactactacgttgaaagttcaccatactagtaagtttatactgtaagctgttcacccgtctagttctaccactcctggatcaactatggacaccaaaagacatgtggttctgtccacttacctcttttcccctaacactttagagaagacacctcccttgtgaaacgtttcgtcgttagggtatactgttcttcataaatagtctattggtgtaaaattatagctgagaccactagcgatcgtgtcattgagaccgtcaaaaccctcgctagtaaaccaaggagatatcttctttgcgcgcgagaccgaagtggagaaaaagtggacgtggtgacagtggaatgtcactaccgacacgggaccatcaacgtcttcttctacttatacgacaagtcgacctgattctcaaaccattatagtccggtcgtataactyaggtaactgttwttaaaggacacgttccaaaagtatatttt

SEQ ID NO: 14 - DNA, 979 bp, Rattus norvegicus
cacgaggcctcgctsgtgctgctgagcctagccactgycatcttcgctgactgtccttcgtccatctgggttcagttccaaggcagctgttacacttttcttcaagtaaccatcaatgtggaaaacatagaggatgtcagaaagcagtgtactgatcacggggcagacctggtaagtatacacaatgaagaagaaaacgcatttatactggacactttacaaaagcgatggaaaggcccggatgatcttctgctaggcatgttttatgacactgatgatgcaagtttcaagtggtttgatcagtcaaatatgacattcgacaagtgggcagatgaggatggtgaggacctagttgacacctgtggttttctgtatgccaagacaggtgaatggagaaaaggaaattgtgaaatgtcttctgtgacrggaacactttgcaaaacagcaatcccatatgacaagaagtatttatcagataaccacattttaatatcgactctggtgatcgctagcacagtgactctggcagttttgggagcggtcatttggttcctctatagaaggagcgcacgctctggcttcacctctttctctcctgcaccacaatcaccttacagtgatggctgtgctctggtagttgcggaagaagatgaatactctgttcagctggactgagagtttgggaacatcagacgagcacactgaacaccttgacaagaaataatttcctatgcaagattgtcatgtaaaatttgccacggaaaactgaaccttttatggtattccttattcttctaacaatattttcatgtattcaatgtgacaaaacataaaccttctgattaaaaggaaaaaaagtaggtttcagaaaaggaactagcacagagctaacttacaggttttcttaagtagttttcatttgagtaaatgaaagctacagtacaataaagctggtaaaacgcaaaaaaaaaaaaaaaaa

SEQ ID NO: 15 - protein, 224 aa, Rattus norvegicus
  VARIANT 5: Xaa = Leu
HEASXVLLSLATXIFADCPSSIWVQFQGSCYTFLQVTINVENIEDVRKQCTDHGADLVSIHNEEENAFILDTLQKRWKGPDDLLLGMFYDTDDASFKWFDQSNMTFDKWADEDGEDLVDTCGFLYAKTGEWRKGNCEMSSVXGTLCKTAIPYDKKYLSDNHILISTLVIASTVTLAVLGAVIWFLYRRSARSGFTSFSPAPQSPYSDGCALVVAEEDEYSVQLD

SEQ ID NO: 16 - protein, 228 aa, Rattus norvegicus
MPHAALSSLVLLSLATAIFADCPSSIWVQFQGSCYTFLQVTINVENIEDVRKQCTDHGADLVSIHNEEENAFILDTLQKRWKGPDDLLLGMFYDTDDASFKWFDQSNMTFDKWADEDGEDLVDTCGFLYAKTGEWRKGNCEMSSVTGTLCKTAIPYDKKYLSDNHILISTLVIASTVTLAVLGAVIWFLYRRSARSGFTSFSPAPQSPYSDGCALVVSEEDEYSVQLD

SEQ ID NO: 17 - DNA, 979 bp, Rattus norvegicus
gtgctccggagcgascacgacgactcggatcggtgacrgtagaagcgactgacaggaagcaggtagacccaagtcaaggttccgtcgacaatgtgaaaagaagttcattggtagttacaccttttgtatctcctacagtctttcgtcacatgactagtgccccgtctggaccattcatatgtgttacttcttcttttgcgtaaatatgacctgtgaaatgttttcgctacctttccgggcctactagaagacgatccgtacaaaatactgtgactactacgttcaaagttcaccaaactagtcagtttatactgtaagctgttcacccgtctactcctaccactcctggatcaactgtggacaccaaaagacatacggttctgtccacttacctcttttcctttaacactttacagaagacactgyccttgtgaaacgttttgtcgttagggtatactgttcttcataaatagtctattggtgtaaaattatagctgagaccactagcgatcgtgtcactgagaccgtcaaaaccctcgccagtaaaccaaggagatatcttcctcgcgtgcgagaccgaagtggagaaagagaggacgtggtgttagtggaatgtcactaccgacacgagaccatcaacgccttcttctacttatgagacaagtcgacctgactctcaaacccttgtagtctgctcgtgtgacttgtggaactgttctttattaaaggatacgttctaacagtacattttaaacggtgccttttgacttggaaaataccataaggaataagaagattgttataaaagtacataagttacactgttttgtatttggaagactaattttcctttttttcatccaaagtcttttccttgatcgtgtctcgattgaatgtccaaaagaattcatcaaaagtaaactcatttactttcgatgtcatgttatttcgaccattttgcgttttttttttttttttt

SEQ ID NO: 18 - DNA, 483 bp, Bos taurus
  misc_feature 43: n = A,T,C or G
gagctagttgacacctgtgcctttttgcacaccaagacaggtngattggaaaaaaggaaactgtgaagtttcttctgtggaaggaaccctttgtaaagcagctatcccatatgaaaagaaatatttatcagataaccgcattttaatatcagctttggtgattgctagcacagtaattctgacagttctgggagcagttgtttggttcttgtacaaaagaagtttggattctggtttcaccacagttttttcagctgcacaccaatcaccttataatgatgactgtgttttagtagttgcagaggaaaacgaatatgatattcaatttaactaagattttggaaatatcagactaagacaaatacctttcagtgattcctctgtaagatttcaatataaaacctgataatgaaaattagtttttatgatatattaccttattccagtaacattcattactcttatgtaaaatcactgatcatg

SEQ ID NO: 19 - DNA, 21 bp, Artificial Sequence
  synthetic primer
gaccatggagcggacatgata

SEQ ID NO: 20 - DNA, 21 bp, Artificial Sequence
  synthetic primer
ggctctaccatctgggtttgt

SEQ ID NO: 21 - DNA, 29 bp, Artificial Sequence
  synthetic primer
cgggatccgactacgaagaccatgacggt

SEQ ID NO: 22 - DNA, 42 bp, Artificial Sequence
  synthetic primer
cgaattcacttacctgtatatttccttttgtatgggatagct

SEQ ID NO: 23 - protein, 210 aa, Homo sapiens
DCPSSTWIQFQDSCYIFLQEAIKVESIEDVRNQCTDHGADMISIHNEEENAFILDTLKKQWKGPDDILLGMFYDTDDASFKWFDNSNMTFDKWTDQDDDEDLVDTCAFLHIKTGEWKKGNCEVSSVEGTLCKTAIPYKRKYLSDNHILISALVIASTVILTVLGAIIWFLYKKHSDSRFTTVFSTAPQSPYNEDCVLVVGEENEYPVQFD

SEQ ID NO: 24 - protein, 208 aa, Rattus norvegicus
DCPSSIWVQFQGSCYTFLQVTINVENIEDVRKQCTDHGADLVSIHNEEENAFILDTLQKRWKGPDDLLLGMFYDTDDASFKWFDQSNMTFDKWADEDGEDLVDTCGFLYAKTGEWRKGNCEMSSVTGTLCKTAIPYDKKYLSDNHILISTLVIASTVTLAVLGAVIWFLYRRSARSGFTSFSPAPQSPYSDGCALVVSEEDEYSVQLD

SEQ ID NO: 25 - protein, 228 aa, Rattus norvegicus; identical to SEQ ID NO: 16

SEQ ID NO: 26 - protein, 228 aa, Mus musculus; identical to SEQ ID NO: 12

SEQ ID NO: 27 - protein, 208 aa, Mus musculus
DCPSSTWVQFQGSCYAFLQVTINVENIEDVRKQCTDHGADMVSIHNEEENAFILDTLQKRWKGPDDLLLGMFYDTDDATFKWYDHSNMTFDKWADQDGEDLVDTCGFLYTKTGEWRKGDCEISSVEGTLCKAAIPYDKKYLSDNHILISTLVIASTVTLAVLGAIIWFLYRRNARSGFTSFSPAPLSPYSDGCALVVAEEDEYAVQLD

SEQ ID NO: 28 - protein, 122 aa, Homo sapiens
CPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWIC

SEQ ID NO: 29 - protein, 124 aa, Homo sapiens
CPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYEWVC

SEQ ID NO: 30 - protein, 132 aa, Homo sapiens
CPEDWGASSRTSLCFKLYAKGKHEKKTWFESRDFCRALGGDLASINNKEEQQTIWRLITASGSYHKLFWLGLTYGSPSEGFTWSDGSPVSYENWAYGEPNNYQNVEYCGELKGDPTMSWNDINCEHLNNWIC

SEQ ID NO: 31 - protein, 130 aa, Homo sapiens
CPSSTWIQFQDSCYIFLQEAIKVESIEDVRNQCTDHGADMISIHNEEENAFILDTLKKQWKGPDDILLGMFYDTDDASFKWFDNSNMTFDKWTDQDDDEDLVDTCAFLHIKTGEWKKGNCEVSSVEGTLC